(19) United States
(12) Patent Application Publication
(10) Pub. No.: US 2010/0063066 A1
(43) Pub. Date: Mar. 11, 2010

(54) RAF INHIBITOR COMPOUNDS AND METHODS OF USE THEREOF

(75) Inventors: Alexandre J. Buckmelter, Superior, CO (US); Joseph P. Lyssikatos, Boulder, CO (US); Greg Miknia, Boulder, CO (US); Li Ren, Boulder, CO (US); Steven Mark Wenglowsky, Boulder, CO (US); Bryson Rast, Boulder, CO (US)

Correspondence Address: VIKSNINS HARRIS & PADYS PLLP, P.O. BOX 111098, ST. PAUL, MN, 55111-1098, US

(73) Assignee: Array Biopharma, Inc., Boulder, CO (US)

(21) Appl. No.: 12/438,967

(22) PCT Filed: Aug. 31, 2007
(86) PCT No.: PCT/US07/77411

§ 371 Date: Oct. 27, 2009

Related U.S. Application Data
(60) Provisional application No. 60/903,456, filed on Feb. 26, 2007.

(30) Foreign Application Priority Data
Aug. 31, 2006 (US) PCT/US2006/033976

(51) Int. Cl. A61K 31/497; C07D 401/14; C07D 471/04; A61K 31/4439; A61P 35/00

(52) U.S. Cl. 514/256; 544/333; 546/115; 514/302

(57) ABSTRACT

Compounds of Formulas (I), (IIA) and (IIIA) are useful for inhibiting Raf kinase and for treating disorders mediated thereby. Methods of using compounds of Formulas (I), (IIA) and (IIIA) and stereoisomers and pharmaceutically acceptable salts thereof, for in vitro, in situ, and in vivo diagnosis, prevention or treatment of such disorders in mammalian cells, or associated pathological conditions are disclosed.

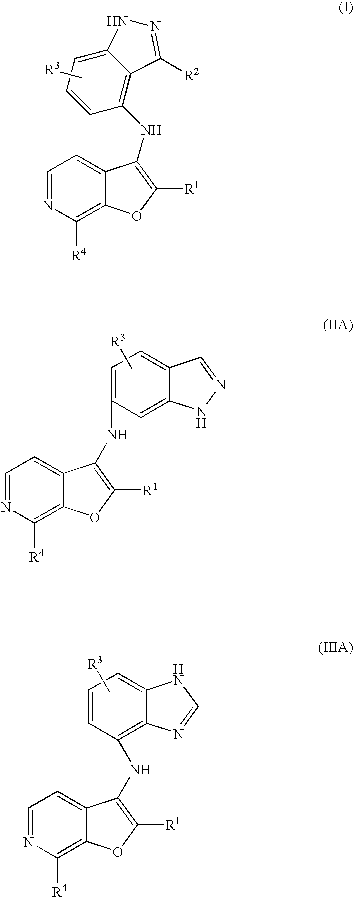

### PRIORITY OF INVENTION

This application claims priority under 35 U.S.C. 119(a) and 35 U.S.C. 365(b) from International PCT Application Number PCT/US2006/033976 that was filed on 31 Aug. 2006, and under 35 U.S.C. 119(e) from U.S. Provisional Application 60/903,456 that was filed on 26 Feb. 2007; both applications are hereby incorporated herein by reference in their entirety.

### BACKGROUND OF THE INVENTION

1. Field of the Invention

Provided are compounds that are inhibitors of Raf kinase, as well as compositions containing these compounds and methods of use thereof. The compounds are useful for inhibiting Raf kinase and for treating disorders mediated thereby. Also provided are methods of using the compounds of the present invention for in vitro, in situ, and in vivo diagnosis or treatment of mammalian cells and/or associated pathological conditions.

2. Description of the State of the Art

The Raf/MEK/ERK (extracellular signal-regulated kinase) kinase cascade is pivotal in transmitting signals from membrane receptors to transcription factors that control gene expression culminating in the regulation of cell cycle progression (Robinson, M J and Cobb, M H (1997) Curr. Opin. Cell Biol., 9:180-186). This cascade can prevent cell death through ERK2 and p90(Rsk) activation and phosphorylation of apoptotic and cell cycle regulatory proteins (Shelton, J G, et al. (2003) Oncogene, 22(16):2478-92). The PI3K/Akt kinase cascade also controls apoptosis and can phosphorylate many apoptotic and cell cycle regulatory proteins. These pathways are interwoven as Akt can phosphorylate Raf and result in its inactivation, and Raf can be required for the anti-apoptotic effects of Akt. Raf is a key serine-threonine protein kinase which participates in the transmission of growth, anti-apoptotic and differentiation messages. These signals can be initiated after receptor ligation and are transmitted to members of the MAP kinase cascade that subsequently activate transcription factors controlling gene expression.

Raf is a multigene family which expresses oncoprotein kinases: A-Raf, B-Raf and C-Raf (also known as Raf-1) (McCubrey, J A, et al., (1998) Leukemia 12(12):1903-1929; Ikawa, et al., (1988) Mol. and Cell. Biol., 8(6):2651-2654; Sithanandam, et al., (1990) Oncogene, 5:1775-1780; Konishi, et al., (1995) Biochem. and Biophys. Res. Comm., 216(2):526-534). All three Raf kinases are functionally present in certain human hematopoietic cells, and their aberrant expression can result in abrogation of cytokine dependency. Their regulatory mechanisms differ because C-Raf and A-Raf require additional serine and tyrosine phosphorylation within the N region of the kinase domain for full activity (Mason et al., (1999) EMBO J., 18:2137-2148), and B-Raf has a much higher basal kinase activity than either A-Raf or C-Raf: The three Raf oncoproteins play critical roles in the transmission of mitogenic and anti-apoptotic signals. It has been shown that B-Raf is frequently mutated in various human cancers (Wan, et al., (2004) Cell, 116:855-867). Development of specific Raf inhibitors may prove efficacious in cancer therapy. The cytoplasmic serine/threonine kinase B-Raf and receptor tyrosine kinases of the platelet-derived growth factor receptor (PDGFR) family are frequently activated in cancer by mutations of an equivalent amino acid. Structural studies have provided important insights into why these very different kinases share similar oncogenic hot spots and why the PDGFR juxtamembrane region is also a frequent oncogenic target (Dibb, N J (2004) Nature Reviews Cancer, 4 (9): 718-27).

Transformation of normal melanocytes into melanoma cells is accomplished by the activation of growth stimulatory pathways, typically leading to cellular proliferation and the inactivation of apoptotic and tumor suppressor pathways. Small molecule inhibitors of proteins in the growth stimulatory pathways are under active investigation, and their application to melanoma patients would represent a treatment strategy to inhibit cell proliferation or induce cell death (Polsky, D., (2003) Oncogene, 22(20):3087-3091; Konopleva, M., et al., (2003) Blood, 102(11):625a).

B-Raf encodes a RAS-regulated kinase that mediates cell growth and malignant transformation kinase pathway activation. Activating B-Raf mutations have been identified in 66% of melanomas and a smaller percentage of many other human cancers. B-Raf mutations also account for the MAP kinase pathway activation common in non-small cell lung carcinomas (NSCLCs), including V600E and other mutations identified as novel, altering residues important in AKT-mediated B-Raf phosphorylation, which suggest that disruption of AKT-induced B-Raf inhibition can play a role in malignant transformation. Although >90% of B-Raf mutations in melanoma involve codon 600 (57 of 60), 8 of 9 B-Raf mutations reported to date in NSCLC are non-V600 (89%; P<10⁻⁷), strongly suggesting that B-Raf mutations in NSCLC are qualitatively different from those in melanoma; thus, there may be therapeutic differences between lung cancer and melanoma in response to RAF inhibitors. Although uncommon, B-Raf mutations in human lung cancers may identify a subset of tumors sensitive to targeted therapy (Brose, M S, et al., (2002) Cancer Research, 62(23):6997-7000).

Raf protein kinases are key components of signal transduction pathways by which specific extracellular stimuli elicit precise cellular responses in mammalian cells. Activated cell surface receptors activate ras/rap proteins at the inner aspect of the plasma membrane, which in turn recruit and activate Raf proteins. Activated Raf proteins phosphorylate and activate the intracellular protein kinases MEK1 and MEK2. In turn, activated MEKs catalyze phosphorylation and activation of p42/p44 mitogen-activated protein kinase (MAPK). A variety of cytoplasmic and nuclear substrates of activated MAPK are known, which directly or indirectly contribute to the cellular response to environmental change.

Small molecule inhibitors of the Raf/MEK/ERK pathway are being developed for anticancer therapy (Thompson et al., (2005) Current Opinion in Pharmacology 5:1-7). Inhibitors of Raf kinases have been suggested for use in disruption of tumor cell growth and hence in the treatment of cancers, e.g. histiocytic lymphoma, lung adenocarcinoma, small cell lung cancer and pancreatic and breast carcinoma; and also in the treatment and/or prophylaxis of disorders associated with neuronal degeneration resulting from ischemic events, including cerebral ischemia after cardiac arrest, stroke and multi-infarct dementia and also after cerebral ischemic events such as those resulting from head injury, surgery and/or during childbirth (neurotrauma). In particular, it has been suggested that B-Raf is the major Raf isoform activated by the neurotrophin, nerve growth factor (NGF), for NGF induced extracellular signaling by kinase activation (York et al., (2000) Mol. and Cell. Biol. 20(21):8069-8083).

PCT Patent Application WO 2007/027855 discloses inter alia a variety of compounds that act as Raf inhibitors. These compounds are said to be useful in the treatment of hyperproliferative diseases such as cancer.

### SUMMARY OF THE INVENTION

In one aspect, the invention relates to compounds that are inhibitors of Raf kinases, in particular inhibitors of B-Raf kinase. Certain hyperproliferative disorders are characterized by the overactivation of Raf kinase function, for example by mutations or overexpression of the protein. Accordingly, the compounds of the invention are useful in the treatment of hyperproliferative disorders such as cancer.

More specifically, one aspect of the present invention provides compounds of Formula I:

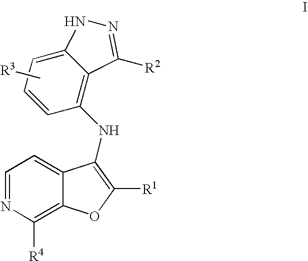

and stereoisomers and pharmaceutically acceptable salts thereof, wherein R¹, R², R³ and R⁴ are as defined herein.

Another aspect of the present invention provides compounds of Formula IIa:

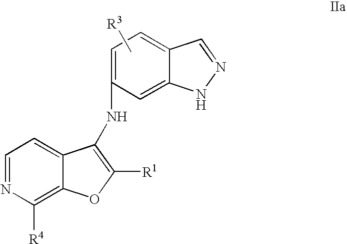

and stereoisomers and pharmaceutically acceptable salts thereof, wherein R¹, R³ and R⁴ are as defined herein.

Another aspect of the present invention provides compounds of Formula II:

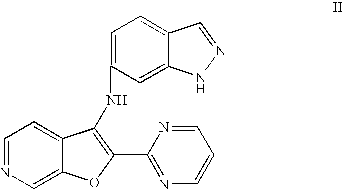

and stereoisomers and pharmaceutically acceptable salts thereof. Another embodiment of the present invention provides compounds of Formula IIIa:

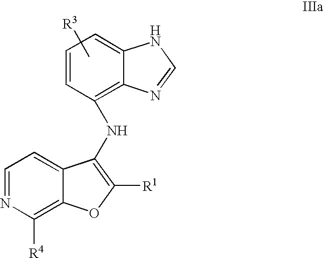

and stereoisomers and pharmaceutically acceptable salts thereof, wherein R¹, R³ and R⁴ are as defined herein.

Another aspect of the present invention provides compounds of Formula III:

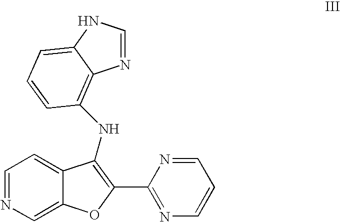

and stereoisomers and pharmaceutically acceptable salts thereof.

Another aspect of the present invention provides methods of preventing or treating a disease or disorder modulated by Raf kinases, comprising administering to a mammal in need of such treatment an effective amount of a compound of this invention or a stereoisomer or pharmaceutically acceptable salt thereof. Examples of such diseases and disorders include, but are not limited to, hyperproliferative disorders (such as cancer, including melanoma and other cancers of the skin), neurodegeneration, cardiac hypertrophy, pain, migraine and neurotraumatic disease.

Another aspect of the present invention provides methods of preventing or treating cancer, comprising administering to a mammal in need of such treatment an effective amount of a compound of this invention, or a stereoisomer or pharmaceutically acceptable salt thereof, alone or in combination with one or more additional compounds having anti-cancer properties.

Another aspect of the present invention provides a method of treating a hyperproliferative disease in a mammal comprising administering a therapeutically effective amount of a compound of this invention to the mammal.

Another aspect of the present invention provides the use of a compound of this invention in the manufacture of a medicament for the treatment of a hyperproliferative disease.

Another aspect of the present invention provides a pharmaceutical composition comprising a compound of this invention or a pharmaceutically acceptable salt thereof, and a pharmaceutically acceptable carrier or excipient.

Another aspect of the present invention provides a process of preparing 6-substituted indazoles.

Another aspect of the present invention includes methods of preparing, methods of separation, and methods of purification of the compounds of this invention.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

### DEFINITIONS

The term “alkyl” as used herein refers to a saturated linear or branched-chain monovalent hydrocarbon radical of carbon atoms, wherein the alkyl radical may be optionally substituted independently with one or more substituents described below.

“Carbocycle” and “carbocyclyl” mean a monovalent non-aromatic, saturated or unsaturated ring, wherein the carbocyclyl radical may be optionally substituted independently with one or more substituents described below.

“Aryl” means a monovalent aromatic hydrocarbon radical of carbon atoms derived by the removal of one hydrogen atom from a single carbon atom of a parent aromatic ring system. Some aryl groups are represented in the exemplary structures as “Ar”.

“Heteroaryl”, “heterocyclyl”, and “heterocycle” all refer to a ring system in which one or more ring atoms are a heteroatom, e.g., nitrogen, oxygen, and sulfur. The heterocyclyl radical may be saturated, partially unsaturated or fully unsaturated. Heterocyclyl groups are optionally substituted independently with one or more substituents described herein.

The term “heteroaryl” also includes aromatic rings containing one or more heteroatoms independently selected from nitrogen, oxygen, and sulfur. Some heteroaryl groups are represented herein as “hetAr”. Heteroaryl groups are optionally substituted independently with one or more substituents described herein.

The terms “treat” or “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. The terms “treating”, “treat”, or “treatment” embrace both preventative, i.e., prophylactic, and palliative treatment.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A “chemotherapeutic agent” is a chemical compound useful in the treatment of cancer. Examples of chemotherapeutic agents include Erlotinib (TARCEVA®, Genentech/OSI Pharm.), Bortezomib (VELCADE®, Millenium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE™ (Cremophor-free), albumin-engineered nanoparticle formulations of paclitaxel (American Pharmaceutical Partners, Schaumberg, Illinois), and TAXOTERE® (doxetaxel; Rhône-Poulenc Rorer, Antony, France); chloranbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); and (x) pharmaceutically acceptable salts, acids and derivatives of any of the above.

The phrase “pharmaceutically acceptable salt,” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The phrase “pharmaceutically acceptable” indicates that the substance or composition must be compatible chemically and/or toxicologically, with the other ingredients comprising a formulation, and/or the mammal being treated therewith.

The compounds of this invention also include other salts of such compounds which are not necessarily pharmaceutically acceptable salts, and which may be useful as intermediates for preparing and/or purifying compounds of this invention and/or for separating enantiomers of compounds of this invention.

RAF INHIBITOR COMPOUNDS

The present invention provides compounds, and pharmaceutical formulations thereof, that are potentially useful in the treatment of diseases, conditions and/or disorders modulated by Raf kinases.

One embodiment of this invention provides compounds of Formula I:

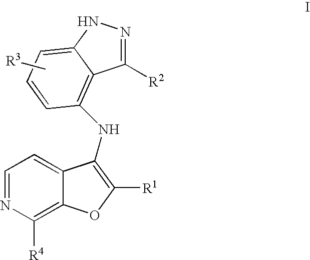

and stereoisomers and pharmaceutically acceptable salts thereof, wherein:

R¹ is selected from H, F, Cl, Br, I, —C(═O)R^{a}, —C(═O)OR^{b}, —C(═O)NR^{b}R^{c}, NR^{b}R^{c}, C₁-C₆ alkyl, C₅-C₈ aryl, C₃-C₈ carbocycle, 5 to 8 member heterocyclyl and 5 to 8 member heteroaryl, wherein said alkyl, aryl, carbocycle, heterocyclyl and heteroaryl are optionally substituted with one or more groups selected from F, Cl, Br, I, R^{d}, —OR^{d}, —COOR^{d}, —C(═O)NR^{d}R^{e}, —N(R^{d})C(═O)R^{e} and —NR^{d}R^{e};

R² is selected from H, F, Cl, Br, I, optionally substituted C₁-C₆ alkyl and —(X) R^{f}, wherein X is O, NH or C(═O), and wherein the alkyl is optionally substituted by one or more groups selected from —OR^{g}, —COOR^{g}, —C(═O)NR^{g}R^{h} and —NR^{g}R^{h};

R³ is one to three substituents independently selected from H, F, Cl, Br, I, CF₃, NH₂ and C₁-C₆ alkyl;

R⁴ is selected from H, F, Cl, Br, I, —NRⁱR^{j} and —ORⁱ;

R^{a} is selected from H, F, Cl, Br, I and C₁-C₆ alkyl, wherein the alkyl is optionally substituted with —NR^{m}Rⁿ or —OR^{m};

R^{b} and R^{c} are selected from H, C₁-C₆ alkyl and —(CR^{k}R^{l})ₜ-heteroaryl, wherein the heteroaryl has 5 to 8 members and the alkyl or heteroaryl are optionally substituted with —(CR^{k}R^{l})ₜNR^{m}Rⁿ or —(CR^{k}R^{l})ₜOR^{m}, or

R^{b} and R^{c} together with the nitrogen to which they are attached form an optionally substituted 5 to 8 member heterocycle or 5 to 8 member heteroaryl, wherein the heterocycle or heteroaryl are optionally substituted with C₁-C₆ alkyl, —(CR^{k}R^{l})ₜNR^{m}Rⁿ or —(CR^{k}R^{l})ₜOR^{m};

R^{d} and R^{e} are independently selected from H or C₁-C₆ alkyl, wherein the alkyl is optionally substituted with —NR^{m}Rⁿ or —OR^{m}, or

R^{d} and R^{e} together with the nitrogen to which they are attached form an optionally substituted 5 to 8 member heterocycle or 5 to 8 member heteroaryl, wherein the heterocycle or heteroaryl are optionally substituted with C₁-C₆ alkyl, —(CR^{k}R^{l})ₜN NR^{m}Rⁿ or —(CR^{k}R^{l})ₜOR^{m};

R^{f} is selected from H, C₁-C₄ alkyl, OR^{m} and —NR^{m}Rⁿ, wherein the alkyl is optionally substituted with one or more groups selected from —OR^{m}, —COOR^{m}, —C(═O)NR^{m}Rⁿ and —NR^{m}Rⁿ;

R^{g} and R^{h} are independently selected from H, C₁-C₆ alkyl or a 5 to 8 member heterocyclyl, wherein the alkyl or heterocyclyl is optionally substituted with C₁-C₆ alkyl, —(CR^{k}R^{l})ₙNR^{m}Rⁿ or —(CR^{k}R^{l})ₜOR^{m};

Rⁱ and R^{j} are H, C₁-C₆ alkyl, —C(═O)R^{m}, —C(═O)OR^{m}, —S(O)₂NR^{m}Rⁿ, wherein the alkyl is optionally substituted with —NR^{m}Rⁿ or —OR^{m};

R^{k} and R^{l} are independently selected from H or C₁-C₆ alkyl;

R^{m} and Rⁿ are H, F, Cl, Br, I, OH, C(═O)OH or C₁-C₆ alkyl, or

R^{m} and Rⁿ together with the atom to which they are attached form an optionally substituted 5 to 8 member heterocycle or 5 to 8 member heteroaryl, wherein the heterocycle or heteroaryl are optionally substituted with F, Cl, Br, I or C₁-C₆ alkyl; and

t is 0, 1, 2, 3 or 4.

In a further embodiment, R³ is one to three substituents independently selected from H, F, Cl, Br, I, CF₃ and C₁-C₆ alkyl.

In certain embodiments, R¹ is a 5 to 8 membered heterocyclyl.

In certain embodiments, R¹ is a 5 membered heterocyclyl optionally substituted with one or more groups selected from F, Cl, Br, I, R^{d}, OR^{d}, COOR^{d}, —C(═O)NR^{d}R^{e}, —N(R^{d})C(═O)R^{c} and NR^{d}R^{e}. In certain embodiments, R¹ is a 5 membered heteroaryl optionally substituted with one or more groups selected from F, Cl, Br, I, R^{d}, OR^{d}, COOR^{d}, —C(═O)NR^{d}R^{e}, —N(R^{d})C(═O)R^{e} and NR^{d}R^{e}. In certain embodiments, R¹ is selected from the structures:

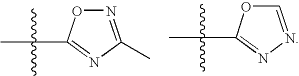

In certain embodiments, R¹ is a 6 membered heterocyclyl optionally substituted with one or more groups selected from F, Cl, Br, I, R^{d}, OR^{d}, COOR^{d}, —C(═O)NR^{d}R^{e}, —N(R^{d})C(═O)R^{e} and NR^{d}R^{e}. In certain embodiments, R¹ is a 6 membered heteroaryl optionally substituted with one or more groups selected from F, Cl, Br, I, R^{d}, OR^{d}, COOR^{d},

—C(═O)NR^{d}R^{e}, —N(R^{d})C(═O)R^{e} and NR^{d}R^{e}. In certain embodiments, R¹ is selected from the structures:

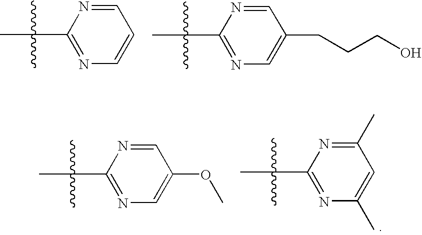

In certain embodiments, R^{d} is C₁-C₆ alkyl. In certain embodiments, R^{d} is methyl.

In certain embodiments, R^{d} is C₁-C₆ alkyl optionally substituted with —OR^{m}. In certain embodiments, R^{m} is H.

In certain embodiments, R¹ is —C(═O)NR^{b}R^{c}. In certain embodiments, R¹ is selected from the structures:

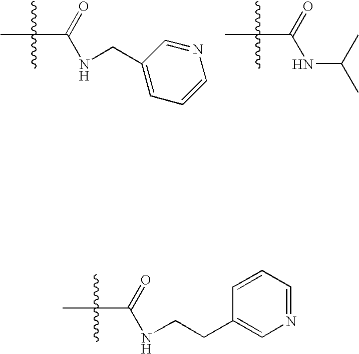

In certain embodiments, R^{b} is —(CR^{k}R^{l})ₜ-heteroaryl, wherein the heteroaryl has 6 members. In certain embodiments, R^{k} and R^{l} are H. In certain embodiments, t is 1 or 2. In certain embodiments, the heteroaryl is pyridine.

In certain embodiments, R^{b} is C₁-C₆ alkyl. In certain embodiments, R^{b} is isopropyl.

In certain embodiments, R^{c} is H.

In certain embodiments, R¹ is —C(═O)OR^{b}. In certain embodiments, R^{b} is ethyl (—CH₂CH₃, “Et”). In certain embodiments, R¹ is —C(═O)OEt.

In certain embodiments, R¹ is H.

In certain embodiments, R² is H.

In certain embodiments, R² is Cl.

In certain embodiments, R² is C₁-C₆ alkyl optionally substituted with one or more groups selected from OR^{g}, COOR^{g}, —C(═O)NR^{g}R^{h} and NR^{g}R^{h}. In certain embodiments, R² is ethyl (—CH₂CH₃). In certain embodiments, R² is ethyl substituted with COOR^{g}. In a further embodiment, R² is —CH₂CH₃C(═O)OH. In certain embodiments, R² is propyl substituted with OR^{g} or NR^{g}R^{h}. In a further embodiment, R² is —CH₂CH₂CH₂OH, —CH₂CH₂CH₂NH₂, —CH₂CH₂CH₂NHCH₃, or —CH₂CH₂CH₂N(CH₃)₂.

In certain embodiments, R² is —(X) R^{f}. In certain embodiments, X is C(═O). In certain embodiments R^{f} is OR^{m}. In certain embodiments, R^{m} is methyl (CH₃).

In certain embodiments, R² is C(═O)OCH₃.

In certain embodiments, R³ is H.

In certain embodiments, R³ is Cl.

In certain embodiments, R³ is F.

In certain embodiment, R³ is C₁-C₆ alkyl. In certain embodiments, R³ is methyl (“Me”, —CH₃).

In certain embodiments, R³ is NH₂.

In certain embodiments, R³ is one substituent. In a further embodiment, R³ is at the 6 position as shown in Formula Ia. In a further embodiment, R³ is at the 7 position as shown in Formula Ib:

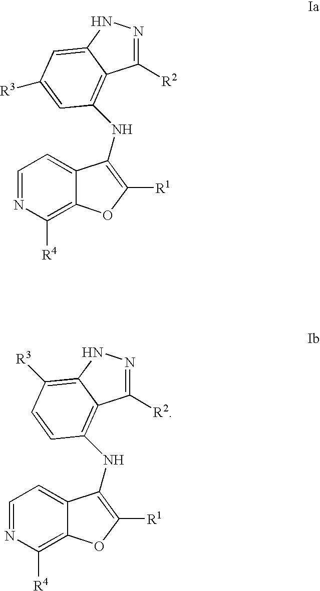

In certain embodiments, R³ is two substituents. In a further embodiment, the two R³ substitutents are at the 6 and 7 position as shown in Formula Ic. In a further embodiment, the two R³ substitutents are at the 5 and 7 position as shown in Formula Id. In a further embodiment, the two R³ substitutents are at the 5 and 6 position as shown in Formula Ie:

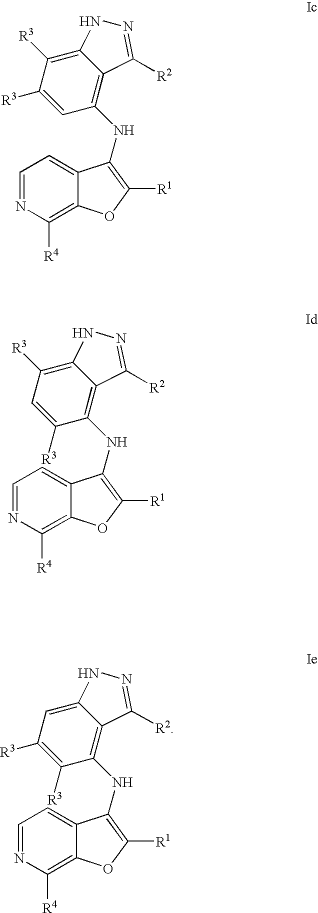

In certain embodiments of Formula Ic, one R³ is methyl and the other R³ is Cl. In certain embodiments of Formula Ic, the 6 position R³ is methyl and the 7 position R³ is Cl.

In certain embodiments, R⁴ is H.

In certain embodiments, R⁴ is Cl.

One embodiment of the present invention provides compounds of Formula I, as defined above, with the proviso that Formula I does not include the compound:

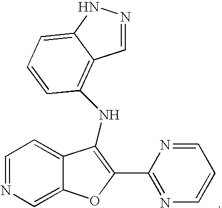

Another embodiment of the present invention provides compounds of Formula IIa:

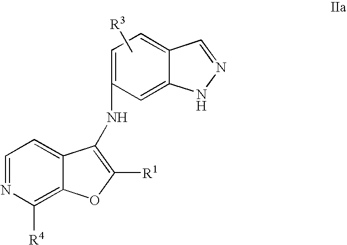

and stereoisomers and pharmaceutically acceptable salts thereof, wherein:

R¹ is selected from H, F, Cl, Br, I, —C(═O)R^{a}, —C(═O)OR^{b}, —C(═O)NR^{b}R^{c}, NR^{b}R^{c}, C₁-C₆ alkyl, C₅-C₈ aryl, C₃-C₈ carbocycle, 5 to 8 member heterocyclyl and 5 to 8 member heteroaryl, wherein said alkyl, aryl, carbocycle, heterocyclyl and heteroaryl are optionally substituted with one or more groups selected from F, Cl, Br, I, R^{d}, —OR^{d}, —COOR^{d}, —C(═O)NR^{d}R^{e}, —N(R^{d})C(═O)R^{c} and —NR^{d}R^{e};

R³ is one to three substituents independently selected from H, F, Cl, Br, I, CF₃, NH₂ and C₁-C₆ alkyl;

R⁴ is selected from H, F, Cl, Br, I, —NRⁱR^{j} and —ORⁱ;

R^{a} is selected from H, F, Cl, Br, I and C₁-C₆ alkyl, wherein the alkyl is optionally substituted with —NR^{m}Rⁿ or —OR^{m};

R^{b} and R^{c} are selected from H, C₁-C₆ alkyl and —(CR^{k}R^{l})ₜ-heteroaryl, wherein the heteroaryl has 5 to 8 members and the alkyl or heteroaryl are optionally substituted with —(CR^{k}R^{l})ₜNR^{m}Rⁿ or —(CR^{k}R^{l})ₜOR^{m}, or

R^{b} and R^{c} together with the nitrogen to which they are attached form an optionally substituted 5 to 8 member heterocycle or 5 to 8 member heteroaryl, wherein the heterocycle or heteroaryl are optionally substituted with C₁-C₆ alkyl, —(CR^{k}R^{l})ₜNR^{m}Rⁿ or —(CR^{k}R^{l})ₜOR^{m};

R^{d} and R^{e} are independently selected from H or C₁-C₆ alkyl, wherein the alkyl is optionally substituted with —NR^{m}Rⁿ or —OR^{m}, or

R^{d} and R^{e} together with the nitrogen to which they are attached form an optionally substituted 5 to 8 member heterocycle or 5 to 8 member heteroaryl, wherein the heterocycle or heteroaryl are optionally substituted with C₁-C₆ alkyl, —(CR^{k}R^{l})ₜNR^{m}Rⁿ or —(CR^{k}R^{l})ₜOR^{m};

Rⁱ and R^{j} are H, C₁-C₆ alkyl, —C(═O)R^{m}, —C(═O)OR^{m}, —S(O)₂NR^{m}Rⁿ, wherein the alkyl is optionally substituted with —NR^{m}Rⁿ or —OR^{m};

R^{k} and R^{l} are independently selected from H or C₁-C₆ alkyl;

R^{m} and Rⁿ are H, F, Cl, Br, I, OH, C(═O)OH or C₁-C₆ alkyl, or

R^{m} and together with the atom to which they are attached form an optionally substituted 5 to 8 member heterocycle or 5 to 8 member heteroaryl, wherein the heterocycle or heteroaryl are optionally substituted with F, Cl, Br, I or C₁-C₆ alkyl; and

t is 0, 1, 2, 3 or 4.

In a further embodiment, R³ is one to three substituents independently selected from H, F, Cl, Br, I, CF₃ and C₁-C₆ alkyl.

In a further embodiment, R³ and R⁴ are H, and R¹ is pyrimidine.

Another embodiment of the present invention provides compounds of Formula II:

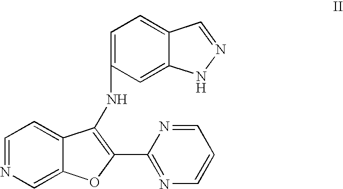

and stereoisomers and pharmaceutically acceptable salts thereof.

Another embodiment of the present invention provides compounds of Formula IIIa:

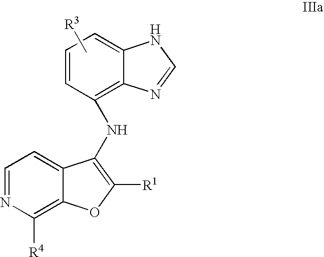

and stereoisomers and pharmaceutically acceptable salts thereof, wherein:

R¹ is selected from H, F, Cl, Br, I, —C(═O)R^{a}, —C(═O)OR^{b}, —C(═O)NR^{b}R^{c}, NR^{b}R^{c}, C₁-C₆ alkyl, C₅-C₈ aryl, C₃-C₈ carbocycle, 5 to 8 member heterocyclyl and 5 to 8 member heteroaryl, wherein said alkyl, aryl, carbocycle, heterocyclyl and heteroaryl are optionally substituted with one or more groups selected from F, Cl, Br, I, R^{d}, —OR^{d}, —COOR^{d}, —C(═O)NR^{d}R^{e}, —N(R^{d})C(═O)R^{e} and —NR^{d}R^{e};

R³ is one to three substituents independently selected from H, F, Cl, Br, I, CF₃, NH₂ and C₁-C₆ alkyl;

R⁴ is selected from H, F, Cl, Br, I, —NRⁱR^{j} and —ORⁱ;

R^{a} is selected from H, F, Cl, Br, I and C₁-C₆ alkyl, wherein the alkyl is optionally substituted with —NR^{m}Rⁿ or —OR^{m};

R^{b} and R^{c} are selected from H, C₁-C₆ alkyl and —(CR^{k}R^{l})ₜ-heteroaryl, wherein the heteroaryl has 5 to 8 members and the alkyl or heteroaryl are optionally substituted with —(CR^{k}R^{l})ₜNR^{m}Rⁿ or —(CR^{k}R^{l})ₜOR^{m}, or

R^{b} and R^{c} together with the nitrogen to which they are attached form an optionally substituted 5 to 8 member heterocycle or 5 to 8 member heteroaryl, wherein the heterocycle or heteroaryl are optionally substituted with C₁-C₆ alkyl, —(CR^{k}R^{l})ₜNR^{m}Rⁿ or —(CR^{k}R^{l})ₜOR^{m};

R^{d} and R^{e} are independently selected from H or C₁-C₆ alkyl, wherein the alkyl is optionally substituted with —NR^{m}Rⁿ or —OR^{m}, or

R^{d} and R^{e} together with the nitrogen to which they are attached form an optionally substituted 5 to 8 member heterocycle or 5 to 8 member heteroaryl, wherein the heterocycle or heteroaryl are optionally substituted with C₁-C₆ alkyl, —(CR^{k}R^{l})ₜNR^{m}Rⁿ or —(CR^{k}R^{l})ₜOR^{m};

Rⁱ and R^{j} are H, C₁-C₆ alkyl, —C(═O)R^{m}, —C(═O)OR^{m}, —S(O)₂NR^{m}Rⁿ, wherein the alkyl is optionally substituted with —NR^{m}Rⁿ or —OR^{m};

R^{k} and R^{l} are independently selected from H or C₁-C₆ alkyl;

R^{m} and Rⁿ are H, F, Cl, Br, I, OH, C(═O)OH or C₁-C₆ alkyl, or

R^{m} and Rⁿ together with the atom to which they are attached form an optionally substituted 5 to 8 member heterocycle or 5 to 8 member heteroaryl, wherein the heterocycle or heteroaryl are optionally substituted with F, Cl, Br, I or C₁-C₆ alkyl; and

t is 0, 1, 2, 3 or 4.

In a further embodiment, R³ is one to three substituents independently selected from H, F, Cl, Br, I, CF₃ and C₁-C₆ alkyl.

In a further embodiment, R³ and R⁴ are H, and R¹ is pyrimidine.

Another embodiment of the present invention provides compounds of Formula III:

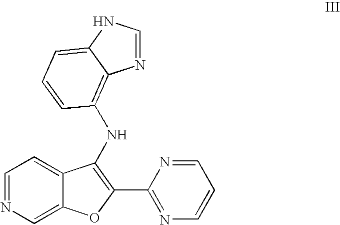

and stereoisomers and pharmaceutically acceptable salts thereof.

The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

The compounds of the present invention may exist in unsolvated as well as solvated forms with pharmaceutically acceptable solvents such as water, ethanol, and the like, and it is intended that the invention embrace both solvated and unsolvated forms.

Synthesis of RAF Inhibitor Compounds

Compounds of the present invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, N.Y. (1967-1999 ed.), or Beilsteins Handbuch der organischen Chemie, 4, Aufl. ed. Springer-Verlag, Berlin, including supplements (also available via the Beilstein online database).

For illustrative purposes, Schemes 1-4 show a general method for preparing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

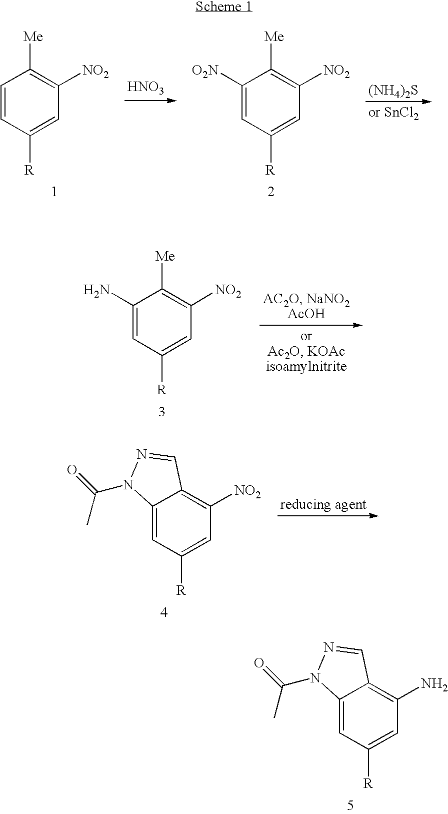

Scheme 1 shows a general scheme for the synthesis of intermediate compound 5, which is useful for the synthesis of compounds of Formula I. As shown in Scheme 1, reaction of a substituted 2-nitrotoluene (R is, for example, H, alkyl, alkoxy, or halogen; see for example Yan-Hong, L., et. al., Molecules 2005, 10, 978-989) with a nitrating reagent such as nitric acid provides a substituted 2,6-dinitrotoluene 2 as a mixture with 2,3- and 2,5-dinitrotoluene (not shown). Such dinitro compounds can be reduced selectively to a monoamine by either ammonium sulfide or tin chloride to give aminotoluenes 3. Conversion of 3 to the corresponding nitroindazoles 4 can be accomplished under either acidic conditions with sodium nitrite or under basic conditions with isoamylnitrite. The nitro group of 4 can be reduced by a variety of methods including ammonium sulfide, tin chloride, iron powder and acetic acid, hydrogenation over palladium on carbon, etc., to give aminoindazole 5.

In another embodiment of the present invention, a process for preparing 6-substituted indazoles is provided. This embodiment includes the process for preparing compounds of Formula 5:

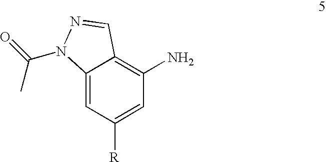

wherein R is H, F, Cl, Br, I, C₁-C₆ alkyl, C₁-C₆ alkoxy, comprising:

(a) reacting a substituted 2-nitrotoluene of Formula 1:

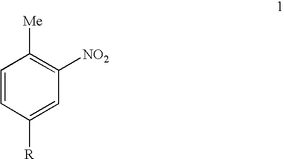

with a nitrating reagent to provide a substituted 2,6-dinitrotoluene of Formula 2:

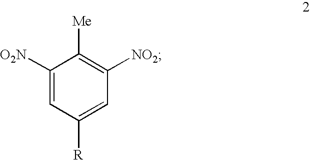

(b) selectively reducing the substituted 2,6-dinitrotoluene of Formula 2 to provide an aminotoluene of Formula 3:

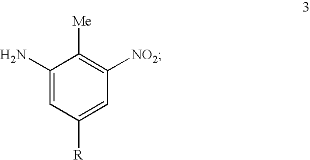

(c) converting the aminotoluene of Formula 3 to the corresponding nitroindazoles of Formula 4:

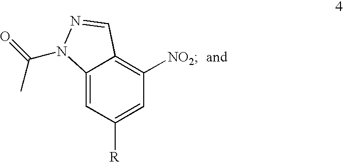

(d) reducing the nitroindazole of Formula 4 to give the 6-substituted indazole of Formula 5.

Step (a) of the reaction includes the reaction with a nitrating agent, such as nitric acid, with or without solvent, to provide a mixture of substituted dinitrotoluenes. Suitable solvents for the nitration include concentrated sulfuric acid and Trifluoroacetic acid, preferably concentrated sulfuric acid. The nitration may take place at a temperature from about 0° C. to about 100° C. The mixture of substituted dinitrotoluenes are preferably separated before performing the next step.

Step (b) of the reaction includes a selective reduction of the substituted 2,6-dinitrotoluene either by ammonium sulfide, iron powder with either acetic acid or ammonium chloride, hydrogenation over palladium on carbon or tin chloride dihydrate.

Step (c) of the reaction includes converting the aminotoluene to the corresponding nitroindazoles by acylation of the aniline with acetic anhydride or acetyl chloride followed by indazole formation with sodium nitrite and acetic acid as solvent or with and organic nitrite such as isoamylnitrite in a suitable solvent, such as dichloromethane, dichloroethane, chloroform or ethyl acetate.

Step (d) of the reaction includes the reduction of the nitroindazole to give the 6-substituted indazole. This reduction may be carried out by a variety of methods known to those skilled in the art, including, for example, ammonium sulfide, tin chloride dihydrate, iron powder with either acetic acid or ammonium chloride, and hydrogenation over palladium on carbon.

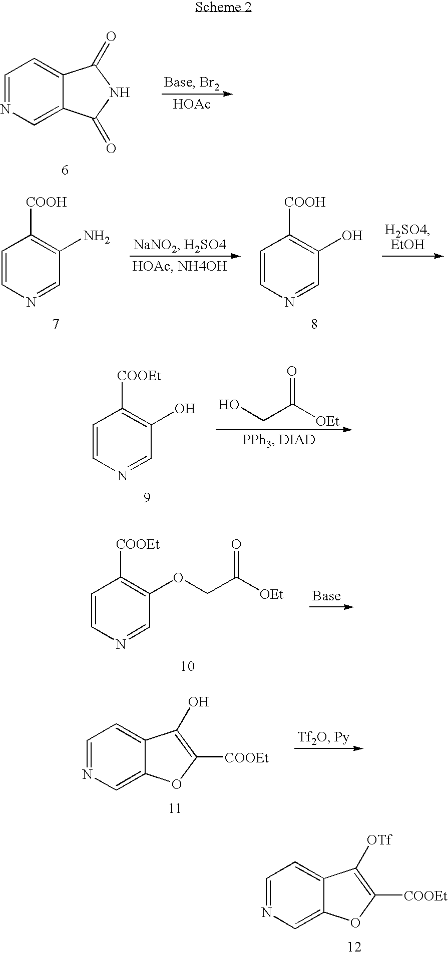

Scheme 2 shows a general scheme for the synthesis of intermediate compound 12, which is useful for the synthesis of compounds of Formula I. According to Scheme 2, treatment of compound 6 with a base such as NaOH in the presence of bromine promotes formation of 3-amino isonicotinic acid 7. Compound 7 can be converted to 3-hydroxyl isonicotinic acid 8 using sodium nitrite and concentrate sulfuric acid. Compound 9 can be obtained from compound 8 via a modified Fisher esterification procedure. Compound 9 is then condensed with ethyl glycolate under Mistunobu conditions to afford hydroxyl ester 10, which can be cyclized to compound 11 in the presence of a base such as NaH. Conversion of 11 to the corresponding triflate 12 can be accomplished with trifluoromethane sulfonic anhydride in the presence of a base such as pyridine.

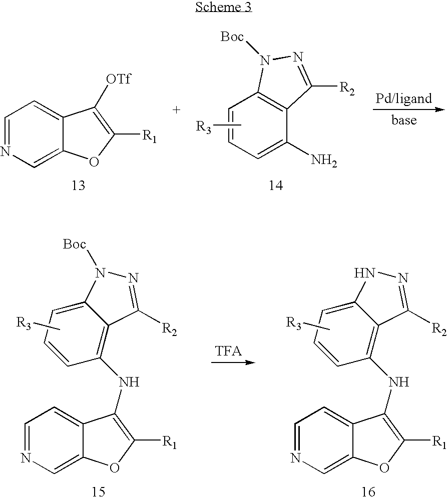

Scheme 3 shows a general scheme for the synthesis of compounds of Formula I. Compound 13 (wherein R¹ is defined above) can be prepared using procedures described in Examples 2 and 9. Compound 14 (wherein R² and R³ are defined above) can be prepared using procedures described in Examples 1, 4 and 7. Triflate 13 and indazole 14 can be coupled under Pd catalyzed Buchwald conditions in the presence of a base to obtain compound 15 and the protecting group can be removed with acids, such as trifluoroacetic acid to afford compound 16.

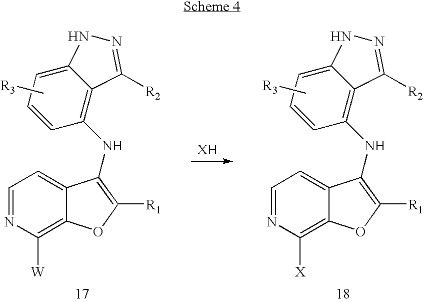

Scheme 4 shows a general scheme for the synthesis of compounds of Formula I. The halofuropyridine precursor 17 can be prepared using procedures described in Example 44. The halofuropyridine precursor 17 (wherein W is a halogen, R¹, R² and R³ are defined above) is placed in steel bomb and reacted with an appropriate nucleophile (XH), wherein X is OR^{y} or NR^{y}R^{z} (wherein R^{y} and R^{z} are selected from H and C₁-C₆ alkyl), at elevated temperatures (150° C.-200° C.) to afford compound 18.

Methods of Separation

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like.

Selection of appropriate methods of separation depends on the nature of the materials involved. For example, boiling point and molecular weight in distillation and sublimation, presence or absence of polar functional groups in chromatography, stability of materials in acidic and basic media in multiphase extraction, and the like. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Also, some of the compounds of the present invention may be atropisomers (e.g., substituted biaryls) and are considered as part of this invention. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and Wilen, S. “Stereochemistry of Organic Compounds,” John Wiley & Sons, Inc., New York, 1994; Lochmuller, C. H., (1975) J. Chromatogr., 113(3):283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: “Drug Stereochemistry, Analytical Methods and Pharmacology,” Irving W. Wainer, Ed., Marcel Dekker, Inc., New York (1993).

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereorneric salts.

Alternatively, by method (2), the substrate to be resolved is reacted with one enantiomer of a chiral compound to form a diastereomeric pair (E. and Wilen, S. “Stereochemistry of Organic Compounds”, John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III. J. Org. Chem., (1982) 47:4165), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (“Chiral Liquid Chromatography” (1989) W. J. Lough, Ed., Chapman and Hall, New York; Okamoto, J. of Chromatogr., (1990) 513:375-378). Enriched or purified enantiomers can be distinguished by methods used to distinguish other chiral molecules with asymmetric carbon atoms, such as optical rotation and circular dichroism.

Biological Evaluation

B-Raf mutant protein 447-717 (V600E) was co-expressed with the chaperone protein Cdc37, complexed with Hsp90 (Roe, et al. Cell, (2004) 116:87-98; Stancato, et al. J. Biol. Chem., (1993) 268:21711-21716).

Determining the activity of Raf in the sample is possible by a number of direct and indirect detection methods (for example, U.S. Patent Publication No. 2004/082014). Activity of human recombinant B-Raf protein may be assessed in vitro by assay of the incorporation of radiolabelled phosphate to recombinant MAP kinase (MEK), a known physiologic substrate of B-Raf, according to U.S. Patent Publication No. 2004/127496 and WO 03/022840. The activity/inhibition of V600E full-length B-Raf was estimated by measuring the incorporation of radiolabeled phosphate from [γ-³³P]ATP into FSBA-modified wild-type MEK (Example 8).

Suitable methods of Raf activity depend on the nature of the sample. In cells, the activity of Raf is on the one hand determined by the amount of the Raf expressed in the cell, and on the other hand by the amount of the activated Raf. The activation of the transcription of the genes coding for Raf protein, in particular B-Raf protein, may be made, for example, by determining the amount of the Raf mRNA. Prior art standard methods comprise for instance the DNA chip hybridization, room temperature PCR, primer extension and RNA protection. Furthermore, the determination of the Raf activity based on the induction or repression of the transcription of the respective Raf gene(s), may also take place by the coupling of the Raf promoter to suitable reporter gene constructs. Examples for suitable reporter genes are the chloramphenicol transferase gene, the green fluorescent protein (GFP) and variants thereof, the luciferase gene and the Renilla gene. The detection of the increase of expression of Raf proteins may however also be made on the protein level, in this case the amount of protein being detected for instance by antibodies directed against Raf protein. The change of the activity of the Raf protein can, however, also be put down to increased or reduced phosphorylation or dephosphorylation of the protein. For instance, the B-Raf kinase is regulated by the phosphorylation of the 599Thr and 602Ser remainders (Zhang B. H. and Guan K. L. EMBO J., (2000) 19:5429). The change of the phosphorylation of B-Raf proteins may be detected, for example, by antibodies directed against phosphorylated threonine or serine.

Since Raf proteins are threonine/serine kinases, the activity of the Raf proteins can also be determined by their enzymatic activity. The protein MEK is for instance a substrate of B-Raf and the degree of the phosphorylation of MEK permits the determination of the B-Raf activity in the sample. In the same way, the phosphorylation of other substrates, as for instance MBP and peptides which are specifically phosphorylated by Raf (Salh, et al., Anticancer Res., (1999) 19:731-740; Bondzi, et al. Oncogene, (2000) 19:5030-5033), of the Raf proteins can be used for determining the respective activity. Since Raf is part of a signal cascade where a series of kinases are respectively phosphorylated and activated by a superordinated kinase, the activity of Raf can also be determined by evaluating the phosphorylation degree of each kinase subordinated to Raf. This so-called map kinase pathway also leads, among other features, to a specific activation of transcription factors and thus to a transcriptional activation of genes, such that the activity of Raf can indirectly be determined by measuring the activity of these target genes.

Administration and Pharmaceutical Formulations

The compounds of the invention may be administered by any convenient route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal. For local immunosuppressive treatment, the compounds may be administered by intralesional administration, including perfusing or otherwise contacting the graft with the inhibitor before transplantation. It will be appreciated that the preferred route may vary with for example the condition of the recipient.

The compounds may be administered in any convenient administrative form, e.g. tablets, powders, capsules, solutions, dispersions, suspensions, syrups, sprays, suppositories, gels, emulsions, patches, etc. Such compositions may contain components conventional in pharmaceutical preparations, e.g. diluents, carriers, pH modifiers, sweeteners, bulking agents, and further active agents. If parenteral administration is desired, the compositions will be sterile and in a solution or suspension form suitable for injection or infusion. Where the compound is administered parenterally, it may be formulated with a pharmaceutically acceptable parenteral vehicle and in a unit dosage injectable form. Where the compound is administered orally, it may be formulated as a pill, capsule, tablet, etc. with a pharmaceutically acceptable carrier or excipient.

A typical formulation is prepared by mixing a compound of the present invention and a carrier or excipient. Most of these carriers or excipients are described in detail in, e.g., Howard C. Ansel et al., Pharmaceutical Dosage Forms and Drug Delivery Systems, (8^{th} Ed. 2004); Alfonso R. Gennaro et al., Remington: The Science and Practice of Pharmacy, (20^{th} Ed. 2000); and Raymond C. Rowe, Handbook of Pharmaceutical Excipients, (5^{th} Ed. 2005). Suitable carriers and excipients are well known to those skilled in the art and include materials such as carbohydrates, waxes, water soluble and/or swellable polymers, hydrophilic or hydrophobic materials, gelatin, oils, solvents, water and the like. The particular carrier or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

One embodiment of the present invention includes a pharmaceutical composition comprising a compound of Formula I, or a stereoisomer or pharmaceutically acceptable salt thereof. In a further embodiment, the present invention provides a pharmaceutical composition comprising a compound of Formula I, or a stereoisomer or pharmaceutically acceptable salt thereof, together with a pharmaceutically acceptable carrier or excipient.

Methods of Treatment with Compounds of the Invention

The invention includes methods of treating or preventing disease or condition by administering one or more compounds of this invention, or a stereoisomer or pharmaceutically acceptable salt thereof. Diseases and conditions treatable according to the methods of this invention include, but are not limited to, cancer, stroke, diabetes, hepatomegaly, cardiovascular disease, Alzheimer's disease, cystic fibrosis, viral disease, autoimmune diseases, atherosclerosis, restenosis, psoriasis, allergic disorders, inflammation, neurological disorders, a hormone-related disease, conditions associated with organ transplantation, immunodeficiency disorders, destructive bone disorders, proliferative disorders, infectious diseases, conditions associated with cell death, thrombin-induced platelet aggregation, chronic myelogenous leukemia (CML), liver disease, pathologic immune conditions involving T cell activation, and CNS disorders in a patient. In one embodiment, a human patient is treated with a compound of Formula I, or a stereoisomer or pharmaceutically acceptable salt thereof, and a pharmaceutically acceptable carrier, adjuvant, or vehicle in an amount to detectably inhibit Raf kinase activity.

In another embodiment, a method of treating or preventing cancer in a mammal in need of such treatment, wherein the method comprises administering to said mammal a therapeutically effective amount of a compound of Formula I, or a stereoisomer or pharmaceutically acceptable salt thereof. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia. Another embodiment of the present invention provides the use of a compound of Formula I, or a stereoisomer or pharmaceutically acceptable salt thereof, in the manufacture of a medicament for the treatment of cancer.

In another embodiment, a method of treating or preventing cardiovascular disease selected from restenosis, cardiomegaly, atherosclerosis, myocardial infarction, or congestive heart failure in a mammal in need of such treatment, wherein the method comprises administering to a mammal a therapeutically effective amount of a pharmaceutical composition comprising a compound of Formula I, or a stereoisomer or pharmaceutically acceptable salt thereof. Another embodiment of the present invention provides the use of a compound of Formula I, or a stereoisomer or pharmaceutically acceptable salt thereof, in the manufacture of a medicament for the treatment of a cardiovascular disease selected from restenosis, cardiomegaly, atherosclerosis, myocardial infarction, or congestive heart failure.

In another embodiment, a method of treating or preventing neurodegenerative disease selected from Alzheimer's disease, Parkinson's disease, amyotrophic lateral sclerosis, Huntington's disease, cerebral ischemia or neurodegenerative disease caused by traumatic injury, glutamate neurotoxicity or hypoxia in a mammal in need of such treatment, wherein the method comprises administering to a mammal a therapeutically effective amount of a pharmaceutical composition comprising a compound of Formula I, or a stereoisomer or pharmaceutically acceptable salt thereof. Another embodiment of the present invention provides the use of a compound of Formula I, or a stereoisomer or pharmaceutically acceptable salt thereof, in the manufacture of a medicament for the treatment of a neurodegenerative disease selected from Alzheimer's disease, Parkinson's disease, amyotrophic lateral sclerosis, Huntington's disease, cerebral ischemia or neurodegenerative disease caused by traumatic injury, glutamate neurotoxicity or hypoxia.

In another embodiment, a method of treating or preventing inflammatory diseases selected from rheumatoid arthritis, psoriasis, contact dermatitis, and delayed hypersensitivity reactions in a mammal in need of such treatment, wherein the method comprises administering to a mammal a therapeutically effective amount of a pharmaceutical composition comprising a compound of Formula I, or a stereoisomer or pharmaceutically acceptable salt thereof. Another embodiment of the present invention provides the use of a compound of Formula I, or a stereoisomer or pharmaceutically acceptable salt thereof, in the manufacture of a medicament for the treatment of a inflammatory diseases selected from rheumatoid arthritis, psoriasis, contact dermatitis, and delayed hypersensitivity reactions.

In another embodiment, a method of treating or preventing a disease or disorder modulated by Raf kinases, comprising administering to a mammal in need of such treatment an effective amount of a compound of Formula I, or a stereoisomer or pharmaceutically acceptable salt thereof. Examples of such diseases and disorders include, but are not limited to, hyperproliferative disorders (such as cancer, including melanoma and other cancers of the skin), neurodegeneration, cardiac hypertrophy, pain, migraine and neurotraumatic disease. In another embodiment of the present invention, a method of treating a hyperproliferative disease in a mammal comprising administering a therapeutically effective amount of the compound of Formula I, or a stereoisomer or pharmaceutically acceptable salt thereof, to the mammal is provided.

Another embodiment of the present invention provides the use of a compound of Formula I, or a stereoisomer or pharmaceutically acceptable salt thereof, in the manufacture of a medicament for the treatment of a hyperproliferative disease.

Combination Therapy

The compounds of this invention and stereoisomers and pharmaceutically acceptable salts thereof may be employed alone or in combination with other therapeutic agents for the treatment of a hyperproliferative disorder (e.g., cancer). In certain embodiments, a compound of this invention is combined in a pharmaceutical combination formulation, or dosing regimen as combination therapy, with a second compound that has anti-hyperproliferative properties or that is useful for treating a hyperproliferative disorder (e.g., cancer). The second compound of the pharmaceutical combination formulation or dosing regimen preferably has complementary activities to the compound of this invention such that they do not adversely affect each other. Such molecules are suitably present in combination in amounts that are effective for the purpose intended. In one embodiment, a composition of this invention comprises a compound of the present invention, or a stereoisomer or pharmaceutically acceptable salt thereof, in combination with a chemotherapeutic agent such as described herein.

In a particular embodiment, in anti-cancer therapy, a compound of the present invention, or a stereoisomer or pharmaceutically acceptable salt thereof, may be combined with other chemotherapeutic, hormonal or antibody agents such as those described herein, as well as combined with surgical therapy and radiotherapy. Combination therapies according to the present invention thus comprise the administration of at least one compound of the present invention, or a stereoisomer or pharmaceutically acceptable salt thereof, and the use of at least one other cancer treatment method. In certain embodiments, combination therapies according to the present invention comprise the administration of at least one compound of this invention, or a stereoisomer or pharmaceutically acceptable salt thereof, and at least one other pharmaceutically active chemotherapeutic agent. The compound(s) of this invention and the other pharmaceutically active chemotherapeutic agent(s) may be administered together in a unitary pharmaceutical composition or separately and, when administered separately this may occur simultaneously or sequentially in any order. Such sequential administration may be close in time or remote in time. The amounts of the compound(s) of this invention and the other pharmaceutically active chemotherapeutic agent(s) and the relative timings of administration will be selected in order to achieve the desired combined therapeutic effect.

### EXAMPLES

In order to illustrate the invention, the following examples are included. However, it is to be understood that these examples do not limit the invention and are only meant to suggest a method of practicing the invention. Persons skilled in the art will recognize that the chemical reactions described may be readily adapted to prepare a number of other Raf inhibitors of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

In the examples described below, unless otherwise indicated all temperatures are set forth in degrees Celsius. Reagents were purchased from commercial suppliers such as Aldrich Chemical Company, Lancaster, TCI or Maybridge, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography was done on a Biotage system (Manufacturer: Dyax Corporation) having a silica gel column or on a silica SepPak cartridge (Waters). ¹H NMR spectra were recorded on a Varian instrument operating at 400 MHz. ¹H-NMR spectra were obtained as CDCl₃, d₆-DMSO, CH₃OD or d₆-acetone solutions (reported in ppm), using chloroform as the reference standard (7.25 ppm). When peak multiplicities are reported, the following abbreviations are used: s (singlet), d (doublet), t (triplet), q (quartet), m (multiplet), br (broadened), dd (doublet of doublets), dt (doublet of triplets). Coupling constants, when given, are reported in Hertz (Hz).

### Example 1

### Preparation of 1-(4-amino-1H-indazol-1-yl)ethanone

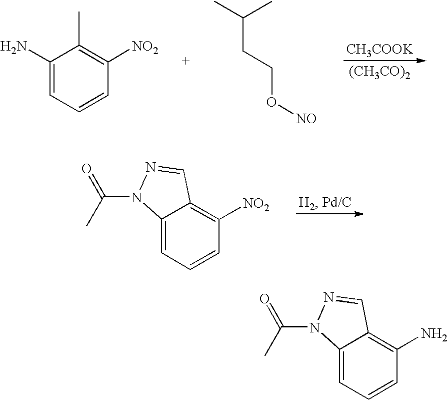

Step A: Preparation of 1-(4-nitro-1H-indazol-1-yl)ethanone: To a solution of 2-methyl-3-nitrobenzenamine (2.00 g, 13.1 mmol) in chloroform (0.8 M) at room temperature was added potassium acetate (1.55 g, 15.8 mmol). The solution was cooled to a temperature of 0° C., and acetic anhydride (3.72 mL, 39.4 mmol) was added dropwise. The suspension was diluted with additional chloroform (20 mL), and the flask was fitted with a condenser and heated to a temperature of 40° C. Isoamyl nitrite (3.52 mL, 26.3 mmol) was added dropwise via an addition funnel and the reaction was heated at reflux for 18 hours. The reaction was cooled and concentrated. The residue was taken up in water and stirred vigorously. The resulting solids (2.4 g, 89% yield) were collected by filtration and used directly in Step B. ¹H NMR (400 MHz, CDCl₃) δ=8.85 (1H, d, J=8.6 Hz); 8.80 (1H, s); 8.29 (1H, d, J=7.8 Hz); 7.71 (1H, m); 2.87 (3H, s).

Step B: Preparation of 1-(4-amino-1H-indazol-1-yl)ethanone: A solution of 1-(4-nitro-1H-indazol-1-yl)ethanone (1.1 g, 5.36 mmol) in ethanol (“EtOH”, 30 mL) was prepared, and 10% Pd/C (0.0571 g, 0.536 mmol) was added. The reaction mixture was purged with N₂ and hydrogenated with H₂ (30 psi) for 3 hours. The reaction was filtered through celite, concentrated and purified by chromatography (1-10% methanol/dichloromethane). The desired product (750 mg, 79% yield) was isolated as an orange solid. ¹H NMR (400 MHz, CDCl₃) δ=8.07 (1H, s); 7.81 (1H, d, J=8.6 Hz); 7.33 (1H, m); 6.56 (1H, d, J=7.8 Hz); 4.17 (2H, br s), 2.76 (3H, s).

### Example 2

### Preparation of 2-(pyrimidin-2-yl)furo[2,3-c]pyridin-3-yl trifluoromethanesulfonate

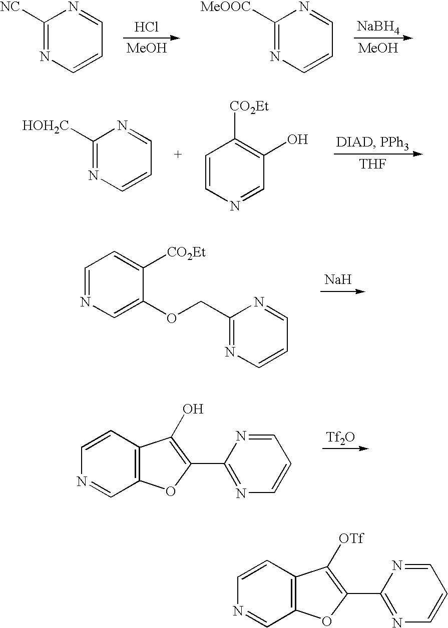

Step A: Preparation of methylpyrimidine-2-carboxylate: HCl gas was bubbled through methanol (“MeOH”, 700 mL) at a temperature of 0° C. for 30 minutes to give a saturated solution. Pyrimidine-2-carbonitrile (21.585 g, 205.38 mmol) was added to this solution, and the mixture was stirred at room temperature for 16 hours and then at a temperature ranging from about 40 to about 50° C. for 3 hours. The reaction mixture was concentrated, and the residue was dissolved in water. The pH was adjusted to about 7.0 using solid NaHCO₃. The aqueous layer was extracted with 20% isopropyl alcohol (“iPrOH”)/dichloromethane (“DCM”) (3×). The combined organics were dried over sodium sulfate, filtered and concentrated under vacuum to give the desired product as white solids (23.0 g, 81%). ¹H NMR (400 MHz, CDCl₃) δ 8.97-8.96 (d, J=4.7 Hz, 2H), 7.53-7.50 (t, J=4.7 Hz, 1H), 4.09 (s, 3H).

Step B: Preparation of pyrimidin-2-ylmethanol: A cold solution (0° C.) of methylpyrimidine-2-carboxylate (659 mg, 4.77 mmol) in EtOH (25 mL) was prepared, and sodium borohydride (181 mg, 4.77 mmol) was added. The reaction mixture was stirred at room temperature for 2 hours. The reaction was quenched with water (5 mL) and concentrated. The crude product was purified using silica gel chromatography to give the desired product as a white solid (154 mg, 30%). ¹H NMR (400 MHz, CDCl₃) δ8.76-8.75 (d, J=4.7 Hz, 2H), 7.27-7.25 (t, J=4.7 Hz, 1H), 4.87 (s, 2H), 4.10 (br s, 1H).

Step C: Preparation of ethyl 3-(pyrimidin-2-ylmethoxy)isonicotinate: A cold solution (−15° C.) of triphenylphospine (14.29 g, 54.49 mmol) in tetrahydrofuran (“THF”, 150 mL) was prepared, and diisopropyl dicarboxylate (“DIAD”, 10.70 mL, 54.49 mmol) was added. The resulting white suspension was stirred at a temperature of −15° C. for 10 minutes before a solution of pyrimidin-2-ylmethanol (5.00 g, 45.41 mmol) in THF (30 mL) was added. The mixture was stirred at a temperature of −15° C. for 10 minutes, and a solution of ethyl 3-hydroxyisonicotinate (7.590 g, 45.41 mmol) in THF (75 mL) was added. The reaction mixture was stirred at a temperature of −15° C. for 15 minutes and then at room temperature overnight. The crude product was concentrated and purified by silica gel chromatography to give the desired product as an orange oil (7.238 g, 61%). MS (APCI-pos) M+1=260.1.

Step D: Preparation of furo[2,3-c]pyridin-3-ol: A cold solution (0° C.) of ethyl 3-(pyrimidin-2-ylmethoxy)isonicotinate (7.238 g, 27.92 mmol) in dimethylformamide (“DMF”, 100 mL) was prepared, and NaH (4.466 g, 111.7 mmol) was added in small portions. The cold bath was removed, and the mixture was stirred at room temperature for 1 hour. The reaction mixture was quenched with water and concentrated. The crude product was purified using silica gel chromatography to give the product as beige colored solids (2.7 g, 45%). MS (APCI-pos) M+1=214.3.

Step E: Preparation of 2-(pyrimidin-2-yl)furo[2,3-c]pyridin-3-yl trifluoromethanesulfonate: A solution of furo[2,3-c]pyridin-3-ol (1.0 equiv) in CH₂Cl₂ at a temperature of 0° C. was prepared, and pyridine (1.5 equiv) and trifluoromethane sulfonic anhydride (“Tf₂O”, 1.2 equiv) were added and stirred for 1 hour. Water was added, and the layers were separated. The aqueous layer was extracted once with chloroform, and the combined organics were dried (sodium sulfate). After filtration, the crude material was concentrated and purified by silica gel chromatography (eluting with ethyl acetate/hexanes) to afford the desired triflate. MS (APCI-pos) M+1=214.3. ¹H NMR (400 MHz, CDCl₃) δ9.11 (s, 1H), 8.97-8.96 (d, J=4.5 Hz, 2H), 8.62-8.61 (d, J=5.7 Hz, 1H), 7.64-7.62 (d, J=4.7 Hz, 1H), 7.41-7.39 (t, J=4.7 Hz, 1H).

### Example 3

### Preparation of N-(1H-indazol-4-yl)-2-(pyrimidin-2-yl)furo[2,3-c]pyridin-3-amine

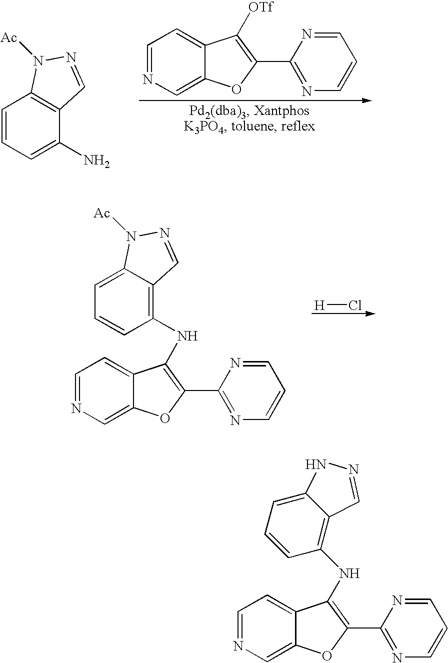

Step A: Preparation of 1-(4-(2-(pyrimidin-2-yl)furo[2,3-c]pyridin-3-ylamino)-1H-indazol-1-yl)ethanone: 1-(4-amino-1H-indazol-1-yl)ethanone (60 mg, 0.34 mmol), 2-(pyrimidin-2-yl)furo[2,3-c]pyridin-3-yl trifluoromethanesulfonate (100 mg, 0.29 mmol), potassium phosphate (98 mg, 0.46 mmol), Xantphos (33 mg, 0.05 mmol) and Pd₂ dba₃ (53 mg, 0.05 mmol) were added to a round bottom flask under argon. The solution was flushed under vacuum with argon, and the solids were suspended in toluene (10 mL). The suspension was degassed repeatedly with argon and heated under argon to a temperature of 110° C. for 18 hours. The solution was cooled and concentrated. The crude product was purified by column chromatography, eluting with 2-5% methanol/dichlormethane to give the desired product (53 mg, 49%).

Step B: Preparation of N-(1H-indazol-4-yl)-2-(pyrimidin-2-yl)furo[2,3-c]pyridin-3-amine: 5 M HCl (0.0249 mL, 0.125 mmol) was added to a suspension of 1-(4-(2-(pyrimidin-2-yl)furo[2,3-c]pyridin-3-ylamino)-1H-indazol-1-yl)ethanone (42 mg, 0.113 mmol) in methanol/dioxane (10 mL, 9:1). The solution was heated at reflux for 18 hours. The reaction was concentrated, poured into saturated sodium bicarbonate solution and extracted with ethyl acetate. The combined organics were dried, filtered and concentrated. The crude product was purified by column chromatography, eluting with 1-6% methanol/dichloromethane to give the desired product (12 mg, 32%). MS (APCI) M+1=329.3.

### Example 4

### Preparation of 1-(4-amino-7-chloro-1H-indazol-1-yl)ethanone and 1-(4-amino-5-chloro-1H-indazol-1-yl)ethanone

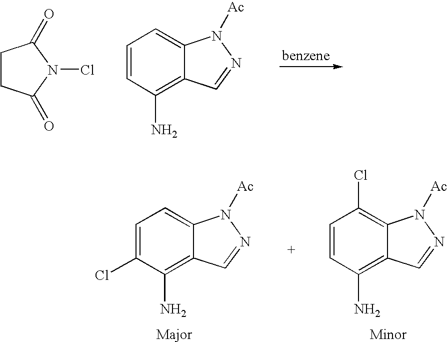

The title compounds were prepared using procedures described by Neale, R. S. et. al, JOC, 1964, 29, 3390.

N-chlorosuccinimide (8.38 g, 63 mmol) was added to a solution of 1-(4-amino-1H-indazol-1-yl)ethanone (10 g, 57 mmol) in benzene (50 mL). The reaction mixture was heated at reflux for 2 hours. The reaction was cooled, poured into water and extracted with ethyl acetate. The combined organics were dried over sodium sulfate, filtered and concentrated. The crude product was taken up in dichloromethane and adsorbed onto a dry silica column. The regioisomers were separated by column chromatography using 20-30% ethyl acetate/hexanes+1% methanol to give 1-(4-amino-5-chloro-1H-indazol-1-yl)ethanone (8 g, 66%) and 1-(4-amino-7-chloro-1H-indazol-1-yl)ethanone (1.3 g, 11%).

1-(4-Amino-5-chloro-1H-indazol-1-yl)ethanone ¹H NMR (400 MHz, CDCl₃) δ=8.04 (1H, s); 7.74 (1H, d, J=8.6 Hz); 7.39 (1H, d, J=8.6 Hz); 4.56 (2H, br s); 2.76 (3H, s).

1-(4-Amino-7-chloro-1H-indazol-1-yl)ethanone ¹H NMR (400 MHz, CDCl₃) δ=8.06 (1H, s), 7.32 (1H, d, J=7.8 Hz); 6.51 (1H, d, J=7.8 Hz); 4.17 (2H, br s), 2.81 (3H, s).

### Example 5

### Preparation of N-(7-chloro-1H-indazol-4-yl)-2-(pyrimidin-2-yl)furo[2,3-c]pyridin-3-amine

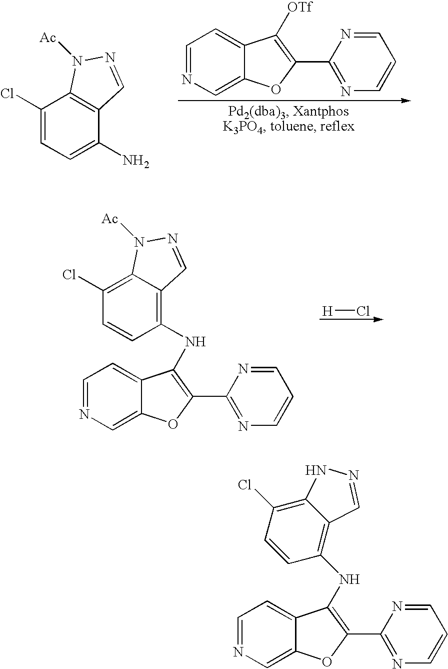

Step A: Preparation of 1-(7-chloro-4-(2-(pyrimidin-2-yl)furo[2,3-c]pyridin-3-ylamino)-1H-indazol-1-yl)ethanone: 1-(4-amino-7-chloro-1H-indazol-1-yl)ethanone (105 mg, 0.5 mmol), 2-(pyrimidin-2-yl)furo[2,3-c]pyridin-3-yl trifluoromethanesulfonate (130 mg, 0.376 mmol), potassium phosphate (170 mg, 0.8 mmol), Xantphos (58 mg, 0.1 mmol) and Pd₂ dba₃ (91 mg, 0.1 mmol) were added to a round bottom flask. The flask was flushed under vacuum with argon, and the solids were suspended in toluene (10 mL). The reaction mixture was degassed repeatedly with argon and heated under argon to a temperature of 110° C. for 18 hours. The solution was cooled and concentrated. The crude product was purified by column chromatography, eluting with 1-8% methanol/dichloromethane to give the desired product (20 mg, 27%). MS (APCI-pos) M+1=405.1, 407.1.

Step B: Preparation of N-(7-chloro-1H-indazol-4-yl)-2-(pyrimidin-2-yl)furo[2,3-c]pyridin-3-amine: 5 M HCl (0.0581 mL, 0.291 mmol) was added to a suspension of 1-(7-chloro-4-(2-(pyrimidin-2-yl)furo[2,3-c]pyridin-3-ylamino)-1H-indazol-1-yl)ethanone (0.056 g, 0.138 mmol) in methanol/dioxane (4 mL, 3:1). The solution was heated to a temperature of 70° C. for 12 hours. The reaction was cooled to room temperature and neutralized with saturated bicarbonate solution to a pH of about 6 to about 7. The solids (30 mg, 59%) were collected by filtration, washed with water, ethyl acetate and dried under high vacuum. MS (APCI-pos) M+1=363.3, 365.3.

### Example 6

### Preparation of N-(5-chloro-1H-indazol-4-yl)-2-(pyrimidin-2-yl)furo[2,3-c]pyridin-3-amine

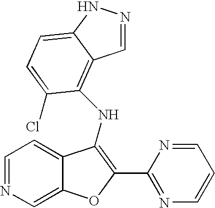

The compound was prepared as described in Example 5 substituting 1-(4-amino-5-chloro-1H-indazol-1-yl)ethanone for 1-(4-amino-7-chloro-1H-indazol-1-yl)ethanone. MS (APCI) M+1=363.3, 365.3.

### Example 7

### Preparation of tert-butyl 4-amino-7-methyl-2H-indazole-2-carboxylate

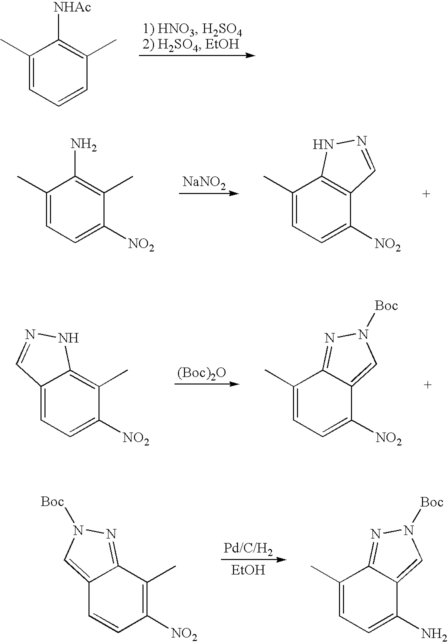

Step A: Preparation of 2,6-dimethyl-3-nitroacetamide: This compound was prepared using procedures described in EP 153855.

Step B: Preparation of 2,6-dimethyl-3-nitroaniline: Concentrated sulfuric acid (30 mL) was added to a solution of 2,6-dimethyl-3-nitroacetamide (14.8 g, 71 mmol) in EtOH (200 mL). The solution was heated at reflux for 96 hours. The reaction was cooled to room temperature, neutralized with 1 M NaOH, poured into brine and extracted with ethyl acetate. The combined organics were dried over sodium sulfate, filtered and concentrated. The crude product was purified by column chromatography, eluting with 2-10% methanol/dichloromethane to give the desired product (11.8 g, 55%).

Step C: Preparation of 7-methyl-4-nitro-1H-indazole and 7-methyl-6-nitro-1H-indazole: Procedure described in Organic Synthesis Collective Volume 3, 1955, p 660. A suspension of 2,6-dimethyl-3-nitroaniline (6.1 g, 37 mmol) in glacial acetic acid (25 mL) was placed in an ice bath, and the internal temperature was monitored with a thermometer. A solution of sodium nitrite (2.5 g, 37 mmol) in water (6.0 mL) was added to this as a single portion. A rapid exotherm was observed, and the internal temperature rose from about 9° C. to about 50° C. upon addition. The resulting suspension was stirred at room temperature for 3 days and then quenched with water. The solids were collected by filtration as a mixture of indazole regioisomers. The crude product was used directly in step D.

Step D: Preparation of tert-butyl 7-methyl-6-nitro-2H-indazole-2-carboxylate and tert-butyl 7-methyl-4-nitro-2H-indazole-2-carboxylate: A mixture (600 mg, 3.11 mmol) of 7-methyl-4-nitro-1H-indazole and 7-methyl-6-nitro-1H-indazole was suspended in dichloromethane (20 mL). Boc₂O (0.678 g, 3.11 mmol) was added to this, followed by triethylamine (0.433 mL, 3.11 mmol). The reaction was stirred at room temperature for 16 hours, and then quenched with water (30 mL). The aqueous layer was extracted with dichloromethane (3×50 mL), and the combined organics were dried, filtered and concentrated. The regioisomers were separated by column chromatography using 20% ethyl acetate/hexanes to give tert-butyl 7-methyl-6-nitro-2H-indazole-2-carboxylate (410 mg, 47%) and tert-butyl 7-methyl-4-nitro-2H-indazole-2-carboxylate (350 mg, 40%).

Tert-butyl 7-methyl-6-nitro-2H-indazole-2-carboxylate: ¹H NMR (400 MHz, CDCl₃) δ=8.6 (1H, s); 7.61 (1H, d, J=9.3 Hz); 7.56 (1H, d, J=9.3 Hz); 2.94 (3H, s); 1.73 (9H, s).

Tert-butyl 7-methyl-4-nitro-2H-indazole-2-carboxylate, ¹H NMR (400 MHz, CDCl₃) δ=9.14 (1H, s); 8.15 (1H, d, J=7.04 Hz); 7.21 (1H, d, J=7.04 Hz); 2.72 (3H, s); 1.75 (9H, s).

Step E: Preparation of tert-butyl 4-amino-7-methyl-2H-indazole-2-carboxylate: 10% Pd/C (0.154 g, 1.44 mmol) was added to a solution of tert-butyl 4-nitro-7-methyl-2H-indazole-2-carboxylate (400 mg, 1.44 mmol, 1 eq.) in MeOH (30 mL). The reaction mixture was purged with N₂, and hydrogenated with H₂ (45 psi) for 16 hours. The reaction mixture was filtered (GF/F paper), and the filtrate was concentrated. The crude product was purified by column chromatography, eluting with 20-30% ethyl acetate/hexanes to give the desired product (287 mg, 80%).

### Example 8

### Preparation of N-(7-methyl-1H-indazol-4-yl)-2-(pyrimidin-2-yl)furo[2,3-c]pyridin-3-amine

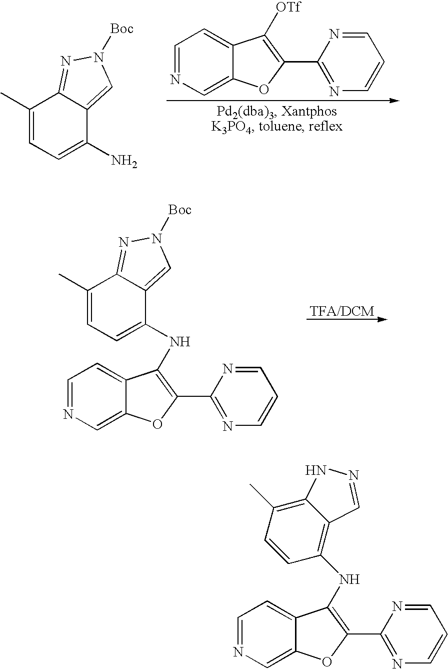

Step A: Preparation of tert-butyl 7-methyl-4-(2-(pyrimidin-2-yl)furo[2,3-c]pyridin-3-ylamino)-4,7-dihydro-2H-indazole-2-carboxylate: A flask was charged with tert-butyl 4-amino-7-methyl-2H-indazole-2-carboxylate (107 mg, 0.43 mmol), 2-(pyrimidin-2-yl)furo[2,3-c]pyridin-3-yl trifluoromethanesulfonate (100 mg, 0.29 mmol), potassium phosphate (98 mg, 0.46 mmol), Xantphos (33 mg, 0.05 mmol) and Pd₂ dba₃ (26 mg, 0.029 mmol). The flask was purged with argon, the solids were suspended in toluene (8 mL) and degassed with argon. The solution was heated to a temperature of 90° C. under argon for 18 hours. The solution was cooled and concentrated. The crude product was purified by column chromatography, eluting with 1-5% methanol/dichloromethane to give the desired product.

Step B: Preparation of N-(7-methyl-1H-indazol-4-yl)-2-(pyrimidin-2-yl)furo[2,3-c]pyridin-3-amine: TFA (2.0 mL) was added dropwise to a solution of tert-butyl 7-methyl-4-(2-(pyrimidin-2-yl)furo[2,3-c]pyridin-3-ylamino)-1H-indazole-1-carboxylate in dichloromethane (2.0 mL). The reaction mixture was stirred at room temperature for 2 hours. The crude mixture was concentrated, and the crude product was purified by flash column chromatography, eluting with 2-8% MeOH/dichloromethane to give the desired product as solids (6 mg; 6% over two steps). MS (APCI-pos) M+1=343.4.

### Example 9

### Preparation of ethyl 3-(trifluoromethylsulfonyloxy)furo[2,3-c]pyridine-2-carboxylate

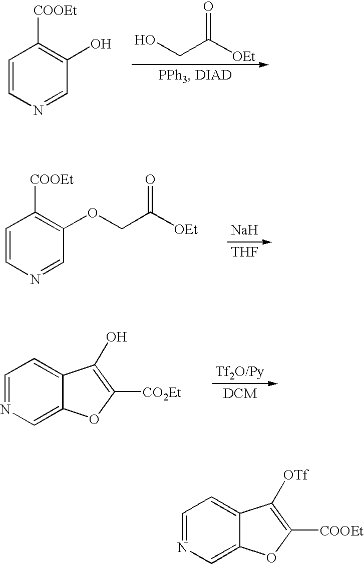

Step A: Preparation of ethyl 3-(2-ethoxy-2-oxoethoxy)isonicotinate: The reaction is carried out in a 3-neck flask (3 L) equipped with an internal thermometer, an addition funnel and a N₂ inlet. Triphenylphosphine (150.6 g, 574 mmol) was dissolved in THF (1 L) and cooled to a temperature of −10° C. DIAD was added dropwise via an addition funnel over 30 minutes. The resulting white suspension was kept at −10° C. for another 30 minutes. Ethyl glycolate (50.84 mL, 526.4 mmol) was added as a solution in THF (500 mL) via the addition funnel at a rate to maintain the internal temperature below −10° C. Upon complete addition, the reaction mixture was kept at a temperature of −10° C. for an additional 30 minutes before a suspension of ethyl 3-hydroxyisonicotinate (80 g, 478.6 mmol) in THF (500 mL) was poured in. The reaction was allowed to warm up slowly to ambient temperature overnight. The reaction mixture was concentrated. The residue was taken up in ethyl acetate (1 L) and extracted with 1N HCl (1×500 mL then 5×250 mL). The aqueous layer was treated with NaHCO₃ to a pH of about 8 and then extracted with ethyl acetate (1 L×3). The combined organics were dried, filtered and concentrated to give the desired product (92.0 g, 76%). MS (APCI-pos) M+1=254.3.

Step B: Preparation of ethyl 3-hydroxyfuro[2,3-c]pyridine-2-carboxylate: Ethyl 3-(2-ethoxy-2-oxoethoxy)isonicotinate (92.0 g, 363 mmol) in THF (300 mL) was added as a solution dropwise via an addition funnel to a suspension of NaH (17.4 g, 436 mmol, 60% suspension in mineral oil) in cold THF (200 mL, 0° C.). Upon complete addition, the reaction mixture was allowed to warm up to ambient temperature overnight. The reaction mixture was cooled to a temperature of 0° C., carefully quenched with ice and then concentrated under vacuum to remove most of the THF. The remaining slurry was diluted with saturated NaHCO₃ (1 L) and stirred for 30 minutes. The solids were collected by filtration, washed with water (200 mL) and ethyl acetate (500 mL), and the solids were set aside. The filtrate was transferred to a separatory funnel, and the layers were separated. The aqueous layer was washed with ethyl acetate (300 mL×3), and the organics were discarded. The aqueous layer was pooled with the solids and carefully acidified to a pH of about 5 with acetic acid (“AcOH”, 100 mL). The resulting yellow solids were collected by filtration and dried under vacuum overnight to give the desired product (63.4 g, 84%). ¹H NMR (400 MHz, CDCl₃) δ 8.9 (s, 1H), 8.5 (d, J=4.8 Hz, 1H), 7.7 (d, J=5.2 Hz, 1H), 4.5 (q, J=7.0 Hz, 2H), 1.5 (t, J=7.0 Hz, 3H) ppm. MS (APCI-pos) M+1=208.2.

Step C: Preparation of ethyl 3-(trifluoromethylsulfonyloxy)furo[2,3-c]pyridine-2-carboxylate: Tf₂O (4.50 mL, 26.6 mmol) was added dropwise to a cold (0° C.) solution of 3-hydroxyfuro[2,3-c]pyridine-2-carboxylate (4.6 g, 22.2 mmol) and pyridine (2.33 mL, 28.9 mmol) in dichloromethane (50 mL). After 2 hours, the reaction mixture was quenched with water, and the aqueous layer was extracted with dichloromethane (50 mL×2). The combined organics were dried, filtered and concentrated. The crude product was purified by flash column chromatography, eluting with hexanes/ethyl acetate (4:1) to give the desired product (6.74 g, 90%). MS (APCI-pos) M+1=340.0.

### Example 10

### Preparation of ethyl 3-(1-acetyl-7-chloro-1H-indazol-4-ylamino)furo[2,3-c]pyridine-2-carboxylate and ethyl 3-(7-chloro-1H-indazol-4-ylamino)furo[2,3-c]pyridine-2-carboxylate

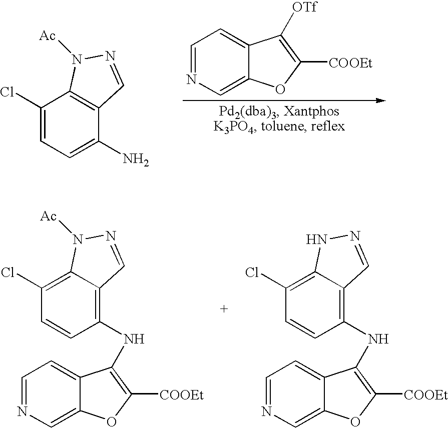

A flask was charged with 1-(4-amino-7-chloro-1H-indazol-1-yl)ethanone (695 mg, 3.3 mmol), ethyl 3-(trifluoromethylsulfonyloxy)furo[2,3-c]pyridine-2-carboxylate (750 mg, 2.21 mmol), potassium phosphate (751 mg, 3.54 mmol), Xantphos (512 mg, 0.884 mmol) and Pd₂ dba₃ (405 mg, 0.442 mmol). The flask was purged with argon and the solids were suspended in toluene (20 mL). The reaction mixture was degassed with argon and heated to a temperature of 90° C. for 24 hours. The reaction was cooled, diluted with dichloromethane, filtered and concentrated. The products, a mixture of ethyl 3-(1-acetyl-7-chloro-1H-indazol-4-ylamino)furo[2,3-c]pyridine-2-carboxylate (200 mg, 22%; APCI-pos, M+1=399.1, 401.1) and ethyl 3-(7-chloro-1H-indazol-4-ylamino)furo[2,3-c]pyridine-2-carboxylate (140 mg, 17%; APCI-pos, M+1=357.2, 359.2), were isolated by column chromatography using 0% to 3% methanol/dichloromethane.

### Example 11

### Preparation of N-(7-chloro-1H-indazol-4-yl)-2-(3-methyl-1,2,4-oxadiazol-5-yl)furo[2,3-c]pyridin-3-amine

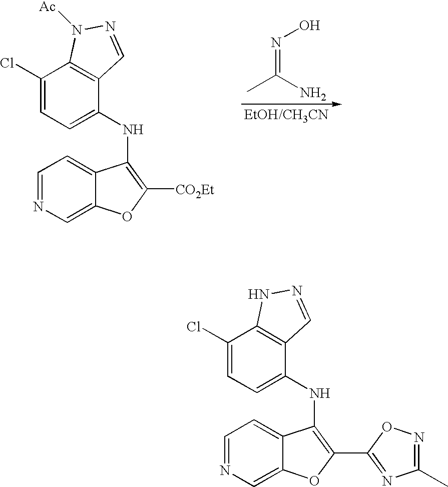

A suspension of ethyl 3-(1-acetyl-7-chloro-1H-indazol-4-ylamino)furo[2,3-c]pyridine-2-carboxylate (0.050 g, 0.125 mmol) and (Z)-N′-hydroxyacetamidine (0.0557 g, 0.752 mmol) in EtOH/acetonitrile (2 mL, 1:1) was sealed and heated to a temperature of 100° C. for 12 hours. The solution was cooled, and the solid was collected by filtration to give the desired product (15 mg, 32%). MS (APCI-pos) M+1=367.2, 369.2.

### Example 12

### Preparation of 3-(7-chloro-1H-indazol-4-ylamino)-N-(pyridin-3-ylmethyl)furo[2,3-c]pyridine-2-carboxamide

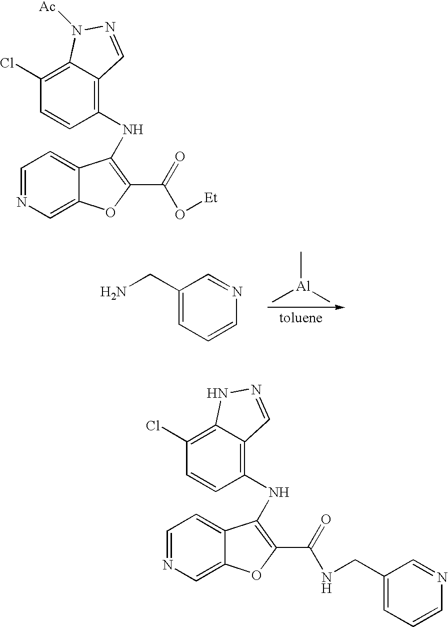

Trimethylaluminum (2.0 M solution in toluene, 0.3134 mL, 0.6269 mmol) was added to a solution of pyridin-3-ylmethanamine (0.06383 mL, 0.6269 mmol) in toluene (5 mL) at a temperature of 0° C. After 30 minutes, ethyl 3-(1-acetyl-7-chloro-1H-indazol-4-ylamino)furo[2,3-c]pyridine-2-carboxylate (0.050 g, 0.1254 mmol) was added, and the solution was heated to a temperature of 80° C. for 3 hours. The reaction was cooled to a temperature of 0° C. and quenched with ice cold water. The emulsion was treated with 30% Rochelle's salt solution. The mixture was extracted with ethyl acetate, dried over sodium sulfate, filtered and concentrated. The crude product was purified by column chromatography using 2-7% methanol/dichloromethane to give the desired product (12 mg, 23%). MS (APCI-pos) M+1=419.1, 421.1.

### Example 13

### Preparation of 3-(7-chloro-1H-indazol-4-ylamino)-N-(2-(pyridin-3-yl)ethyl)furo[2,3-c]pyridine-2-carboxamide

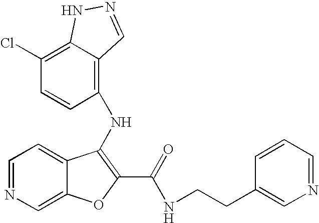

The compound was prepared as described in Example 12 substituting 3-aminoethyl pyridine for pyridine-3-ylmethanamine. MS (APCI) M+1=433.1, 435.1.

### Example 14

### Preparation of N-(7-chloro-1H-indazol-4-yl)-2-(1,3,4-oxadiazol-2-yl)furo[2,3-c]pyridin-3-amine

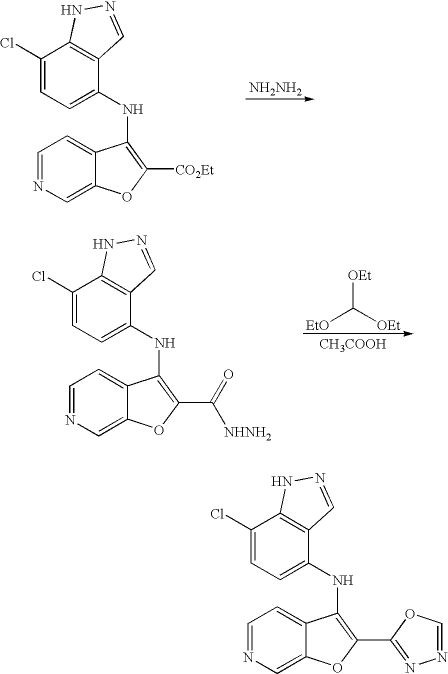

Step A: Preparation of 3-(7-chloro-1H-indazol-4-ylamino)furo[2,3-c]pyridine-2-carbohydrazide: Hydrazine (0.1334 mL, 4.204 mmol) was added to a suspension of ethyl 3-(7-chloro-1H-indazol-4-ylamino)furo[2,3-c]pyridine-2-carboxylate (0.150 g, 0.4204 mmol) in ethanol (2 mL). The reaction vessel was sealed and heated to a temperature of 90° C. for 18 hours. The reaction was cooled and the solid was collected by filtration to give the desired product (65 mg, 45%). MS (APCI) M+1=343.1, 345.1.

Step B: Preparation of N-(7-chloro-1H-indazol-4-yl)-2-(1,3,4-oxadiazol-2-yl)furo[2,3-c]pyridin-3-amine: A suspension of 3-(7-chloro-1H-indazol-4-ylamino)furo[2,3-c]pyridine-2-carbohydrazide (90 mg, 0.26 mmol) in triethyl orthoformate (4 mL) containing acetic acid (0.5 mL) was sealed and heated to a temperature of 130° C. for 30 minutes in a Biotage Initiator microwave reactor. The solution was cooled and concentrated. The crude product was purified by column chromatography using 2-5% methanol/dichloromethane to give the desired product (21 mg, 23%). MS (APCI-pos) M+1=353.2, 355.2

### Example 15

### Preparation of 3-chloro-N-(2-(pyrimidin-2-yl)furo[2,3-c]pyridin-3-yl)-1H-indazol-4-amine

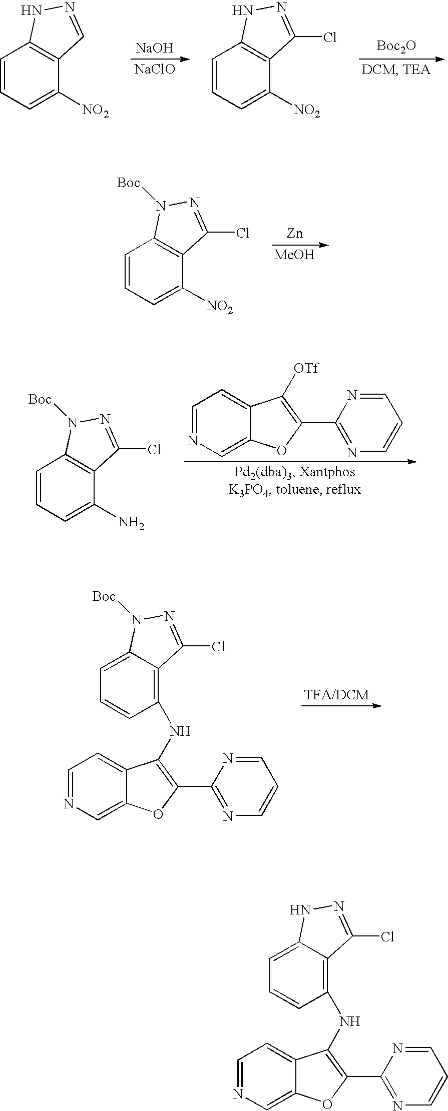

Step A: Preparation of 3-chloro-4-nitro-1H-indazole: 4-Nitro-1H-indazole (1.0 g, 6.13 mmol) was added to a solution of NaOH (0.981 g, 24.5 mmol) in H₂O (30 mL), and the mixture was heated to a temperature of 40° C. until a red solution was formed. The reaction was cooled to a temperature of 0° C. before NaClO (11.1 g, 6.15% wt commercial CHLOROX solution) was added. The cold bath was removed, and the reaction was stirred at room temperature for 60 hours. The pH was adjusted to about 7 with 1 N HCl. The aqueous layer was extracted with ethyl acetate (100 mL×3), and the combined organics were dried, filtered and concentrated. The crude product was purified by flash column chromatography, eluting with hexanes/ethyl acetate (2:1) to give the desired product (1.0 g, 83%). MS (APCI-neg) M−1=196.2, 198.2.

Step B: Preparation of tert-butyl 3-chloro-4-nitro-1H-indazole-1-carboxylate: Triethylamine (0.212 mL, 1.52 mmol) was added to a suspension of 3-chloro-4-nitro-1H-indazole (0.3 g, 1.52 mmol) in dichloromethane (50 mL). Boc₂O (0.325 g, 1.49 mmol) was then added. The reaction was stirred at room temperature for 16 hours and then quenched with water (30 mL). The aqueous layer was extracted with dichloromethane (50 mL×3), and the combined organics were dried, filtered and concentrated. The crude product (0.47 g) was used in step C without purification.

Step C: Preparation of tert-butyl 4-amino-3-chloro-1H-indazole-1-carboxylate: Zn dust (0.93 g, 14.3 mmol) was added to a suspension of tert-butyl 3-chloro-4-nitro-1H-indazole-1-carboxylate (0.425 g, 1.428 mmol) in MeOH/saturated aqueous NH₄Cl (10 mL, 1:1). The reaction was stirred at room temperature for 16 hours. The remaining Zn was removed by filtration, and the filter cake was washed with ethyl acetate. The organic layer was separated, and the aqueous layer was extracted with ethyl acetate (50 mL×3). The combined organics were dried, filtered and concentrated. The crude product was purified by flash column chromatography, eluting with hexanes/ethyl acetate (20:1) to give the desired product (0.26 g, 68%).

Step D: Preparation of tert-butyl 3-chloro-4-(2-(pyrimidin-2-yl)furo[2,3-c]pyridin-3-ylamino)-1H-indazole-1-carboxylate: 2-(Pyrimidin-2-yl)furo[2,3-c]pyridin-3-yl trifluoromethanesulfonate (0.12 g, 0.348 mmol) and tert-butyl 4-amino-3-chloro-1H-indazole-1-carboxylate (0.093 g, 0.348 mmol) were suspended in toluene (5 mL) and degassed with argon for 15 minutes. Xantphos (0.040 g, 0.070 mmol), Pd₂(dba)₃ (0.032 g, 0.035 mmol) and K₃PO₄ (0.162 g, 0.765 mmol) were added to this. The reaction mixture was degassed for another 15 minutes and then heated at reflux under argon overnight. The reaction mixture was filtered (GF/F paper), and the filtrate was purified by flash column chromatography, eluting with hexanes/ethyl acetate (1:1), hexanes/ethyl acetate (1:2) to give the desired product (0.11 g, 68%). MS (APCI-pos) M+1=462.9, 464.9.

Step E: Preparation of 3-chloro-N-(2-(pyrimidin-2-yl)furo[2,3-c]pyridin-3-yl)-1H-indazol-4-amine: Trifluoracetic acid (“TFA”, 2.0 mL) was added dropwise to a suspension of tert-butyl 3-chloro-4-(2-(pyrimidin-2-yl)furo[2,3-c]pyridin-3-ylamino)-1H-indazole-1-carboxylate (0.11 g, 0.24 mmol) in dichloromethane (2.0 mL). The reaction mixture was stirred at room temperature for 1 hour. The crude mixture was concentrated, and the residue was neutralized to a pH of about 7 with saturated aqueous NaHCO₃ (5.0 mL). The resulting solids (0.070 g, 81%) were collected by filtration, washed sequentially with water (˜20 mL), ethyl acetate (˜20 mL) and dried in vacuo. MS (APCI-pos) M+1=363.3, 365.3.

### Example 16

### Preparation of 3-ethyl-N-(2-(pyrimidin-2-yl)furo[2,3-c]pyridin-3-yl)-1H-indazol-4-amine

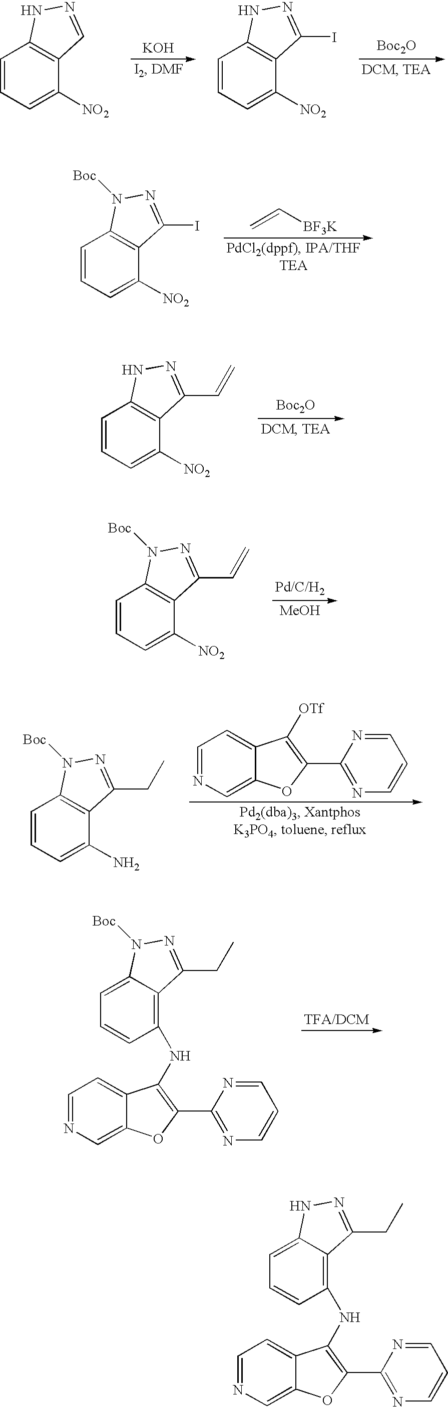

Step A: Preparation of 3-iodo-4-nitro-1H-indazole: Powdered KOH (10.6 g, 195 mmol) was added to a solution of 4-nitro-1H-indazole (3.86 g, 23.7 mmol) in DMF (50 mL) at 0° C. I₂ (24.0 g, 94.6 mmol) in DMF (80 mL) was then added dropwise via an addition funnel. The reaction mixture was left at room temperature for 40 hours. The excess I₂ was carefully quenched with 10% aqueous Na₂S₂O₃, and the pH was adjusted to about 7 with saturated aqueous NaHCO₃. The reaction mixture was concentrated to remove DMF, and the residue was diluted with water. The resulting solids (6.5 g, 95%) were collected by filtration and dried in vacuo.

Step B: Preparation of tert-butyl 3-iodo-4-nitro-1H-indazole-1-carboxylate: Triethylamine (0.67 mL, 4.81 mmol) was added to a suspension of 3-iodo-4-nitro-1H-indazole (1.07 g, 3.70 mmol) in dichloromethane (50 mL), followed by addition of Boc₂O (0.97 g, 4.44 mmol). The reaction was stirred at room temperature for 30 minutes and then quenched with water (30 mL). The aqueous layer was extracted with dichloromethane (50 mL×3), and the combined organics were dried, filtered and concentrated. The crude product was purified by flash column chromatography, eluting with hexanes/ethyl acetate (20:1) to give the desired product (1.1 g, 77%).

Step C: Preparation of 4-nitro-3-vinyl-1H-indazole: Tert-butyl 3-iodo-4-nitro-1H-indazole-1-carboxylate (0.11 g, 0.28 mmol) and potassium vinyltrifluoroborate (0.11 g, 0.848 mmol) were suspended in isopropanol/THF (4:1, 10 mL), and the mixture was degassed with argon for 15 minutes. PdCl₂(dppf)dcm (0.023 g, 0.028 mmol), triethyl amine (0.12 mL, 0.85 mmol) were added, and the reaction mixture was degassed for another 15 minutes. The reaction mixture was then heated to a temperature of 90° C. under argon for 40 hours. The reaction mixture was filtered (GF/F paper), and the filtrate was purified by flash column chromatography, eluting with hexanes/ethyl acetate (4:1), hexanes/ethyl acetate (1:2) to give the desired product (0.033 g, 62%).

Step D: Preparation of tert-butyl 4-nitro-3-vinyl-1H-indazole-1-carboxylate: Triethylamine (0.032 mL, 0.23 mmol) was added to a suspension of 4-nitro-3-vinyl-1H-indazole (0.033 g, 0.17 mmol) in dichloromethane (20 mL), followed by Boc₂O (0.046 g, 0.21 mmol) addition. The reaction was stirred at room temperature for 2 hours and then quenched with water (20 mL). The aqueous layer was extracted with dichloromethane (20 mL×3), and the combined organics were dried, filtered and concentrated. The crude product was purified by flash column chromatography, eluting with hexanes/ethyl acetate (20:1) to give the desired product (0.036 g, 71%).

Step E: Preparation of tert-butyl 4-amino-3-ethyl-1H-indazole-1-carboxylate: 10% Pd/C (0.013 g, 0.012 mmol) was added to a solution of tert-butyl 4-nitro-3-vinyl-1H-indazole-1-carboxylate (0.036 g, 0.12 mmol) in a mixture of MeOH/ethyl acetate (1:4, 20 mL). The reaction mixture was purged with N₂ and hydrogenated with H₂ (14 psi) for 16 hours. The reaction mixture was filtered (GF/F paper), and the filtrate was concentrated to give the desired product (0.032 g, 98%). MS (APCI-pos) M+1=261.8.

Step F: Preparation of tert-butyl 3-ethyl-4-(2-(pyrimidin-2-yl)furo[2,3-c]pyridin-3-ylamino)-1H-indazole-1-carboxylate: 2-(Pyrimidin-2-yl)furo[2,3-c]pyridin-3-yl trifluoromethanesulfonate (0.043 g, 0.125 mmol) and tert-butyl 4-amino-3-ethyl-1H-indazole-1-carboxylate (0.034 g, 0.131 mmol) were suspended in toluene (5 mL) and degassed with argon for 15 minutes. Xantphos (0.014 g, 0.025 mmol), Pd₂(dba)₃ (0.011 g, 0.013 mmol) and K₃PO₄ (0.058 g, 0.27 mmol) were added. The reaction mixture was degassed for another 15 minutes and then heated at reflux under argon overnight. The cooled reaction mixture was filtered (GF/F paper), and the filtrate was purified by flash column chromatography, eluting with hexanes/ethyl acetate (2:1), hexanes/ethyl acetate (1:1) to give the desired product (0.037 g, 65%). MS (APCI-pos) M+1=457.0.

Step G: Preparation of 3-ethyl-N-(2-(pyrimidin-2-yl)furo[2,3-c]pyridin-3-yl)-1H-indazol-4-amine: TFA (2.0 mL) was added dropwise to a suspension of tert-butyl 3-ethyl-4-(2-(pyrimidin-2-yl)furo[2,3-c]pyridin-3-ylamino)-1H-indazole-1-carboxylate (0.037 g, 0.081 mmol) in dichloromethane (2.0 mL). The reaction mixture was stirred at room temperature for 1 hour. The crude mixture was concentrated, and the residue was neutralized to a pH of about 7 with saturated aqueous NaHCO₃ (5.0 mL). The aqueous layer was extracted with dichloromethane (20 mL×3), and the combined organics were dried, filtered and concentrated. The crude product was purified by flash column chromatography, eluting with hexanes/ethyl acetate (1:4) to give the desired product (0.025 g, 87%). MS (APCI-pos) M+1=357.4.

### Example 17

### Preparation of 3-(4-(2-(pyrimidin-2-yl)furo[2,3-c]pyridin-3-ylamino)-1H-indazol-3-yl)propan-1-ol

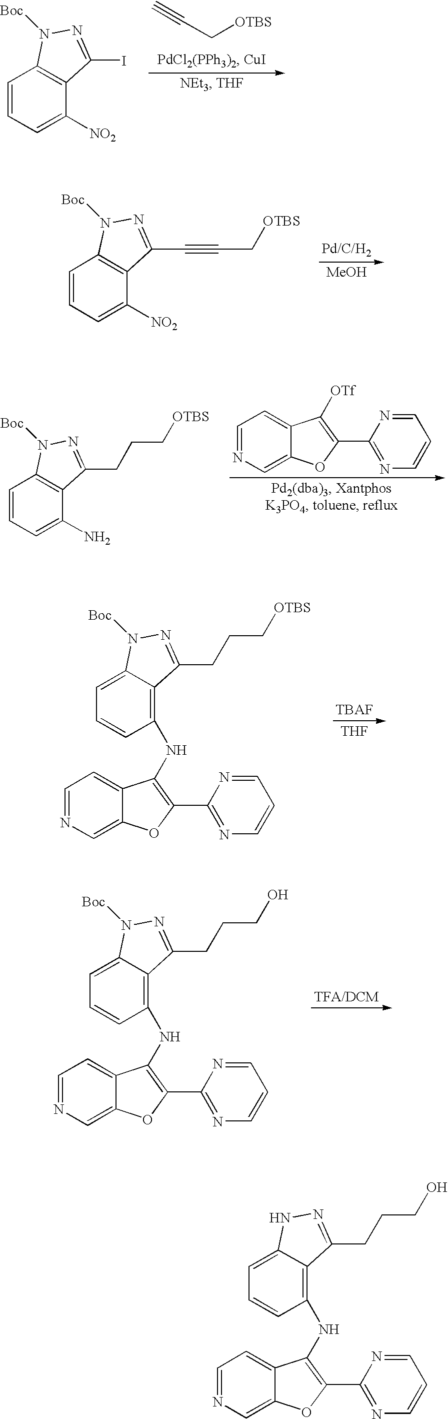

Step A: Preparation of tert-butyl 3-(3-(tert-butyldimethylsilyloxy)prop-1-ynyl)-4-nitro-1H-indazole-1-carboxylate: PdCl₂(PPh₃)₂ (0.162 g, 0.23 mmol) and CuI (0.11 g, 0.576 mmol) were added to a solution of tert-butyl 3-iodo-4-nitro-1H-indazole-1-carboxylate (1.12 g, 2.88 mmol), tert-butyldimethyl(prop-2-ynyloxy)silane (0.88 g, 5.18 mmol), and triethylamine (4 mL) in THF (20 mL). The mixture was purged with argon for 15 minutes and then stirred at room temperature under argon for 16 hours. The reaction mixture was filtered (GF/F paper), and the filtrate was purified by flash column chromatography, eluting with hexanes/ethyl acetate (20:1) to give the desired product (0.64 g, 51%).

Step B: Preparation of tert-butyl 4-amino-3-(3-(tert-butyldimethylsilyloxy)propyl)-1H-indazole-1-carboxylate: 10% Pd/C (0.158 g, 0.148 mmol) was added to a solution of tert-butyl 3-(3-(tert-butyldimethylsilyloxy)prop-1-ynyl)-4-nitro-1H-indazole-1-carboxylate (0.64 g, 1.48 mmol) in a mixture of MeOH/ethyl acetate (1:4, 100 mL). The reaction mixture was purged with N₂ and hydrogenated with H₂ (30 psi) for 16 hours. The reaction mixture was filtered (GF/F paper) and the filtrate was concentrated to give the desired product (0.54 g, 89%). MS (APCI-pos) M+1=405.9.

Step C: Preparation of tert-butyl 3-(3-(tert-butyldimethylsilyloxy)propyl)-4-(2-(pyrimidin-2-yl)furo[2,3-c]pyridin-3-ylamino)-1H-indazole-1-carboxylate: 2-(Pyrimidin-2-yl)furo[2,3-c]pyridin-3-yl trifluoromethanesulfonate (0.43 g, 1.25 mmol) and tert-butyl 4-amino-3-(3-(tert-butyldimethylsilyloxy)propyl)-1H-indazole-1-carboxylate (0.53 g, 1.31 mmol) were suspended in toluene (25 mL) and degassed with argon for 15 minutes. Xantphos (0.144 g, 0.25 mmol), Pd₂(dba)₃ (0.114 g, 0.13 mmol) and K₃PO₄ (0.58 g, 2.74 mmol) were added. The reaction mixture was degassed for another 15 minutes and then heated at reflux under argon overnight. The reaction mixture was filtered (GF/F paper), and the filtrate was purified by flash column chromatography, eluting with hexanes/ethyl acetate (4:1) and hexanes/ethyl acetate (1:1) to give the desired product (0.33 g, 43%). MS (APCI-pos) M+1=601.1.

Step D: Preparation of tert-butyl 3-(3-hydroxypropyl)-4-(2-(pyrimidin-2-yl)furo[2, 3-6]pyridin-3-ylamino)-1H-indazole-1-carboxylate: 1.0 M of tetra-butyl ammonium fluoride solution in THF (0.21 mL, 0.21 mmol) was added to a solution of tert-butyl 3-(3-(tert-butyldimethylsilyloxy)propyl)-4-(2-(pyrimidin-2-yl)furo[2,3-c]pyridin-3-ylamino)-1H-indazole-1-carboxylate (0.082 g, 0.14 mmol) in THF (5.0 mL). The reaction mixture was stirred at room temperature for 1 hour before quenching with water (5.0 mL). The aqueous layer was extracted with ethyl acetate (20 mL×3), and the combined organics were dried, filtered and concentrated. The crude product was purified by flash column chromatography, eluting with hexanes/ethyl acetate (1:4), ethyl acetate to give the desired product (0.044 g, 66%). MS (APCI-pos) M+1=487.0.

Step E: Preparation of 3-(4-(2-(pyrimidin-2-yl)furo 1H-indazol-3-yl)propan-1-ol: TFA (2.0 mL) was added dropwise to a solution of tert-butyl 3-(3-hydroxypropyl)-4-(2-(pyrimidin-2-yl)furo[2,3-c]pyridin-3-ylamino)-1H-indazole-1-carboxylate (0.044 g, 0.090 mmol) in dichloromethane (2.0 mL). The reaction mixture was stirred at room temperature for 3 hours. The crude mixture was concentrated. The residue was taken up in dichloromethane (4.0 mL) and treated with triethylamine (1 mL) for 30 minutes. The reaction mixture was concentrated and the solids (0.030 g, 86%) were collected by filtration, washed with water (˜20 mL) and dried in vacuo. MS (APCI-pos) M+1=387.3.

### Example 18

### Preparation of 3-(3-(dimethylamino)propyl)-N-(2-(pyrimidin-2-yl)furo[2,3-c]pyridin-3-171)-1H-indazol-4-amine

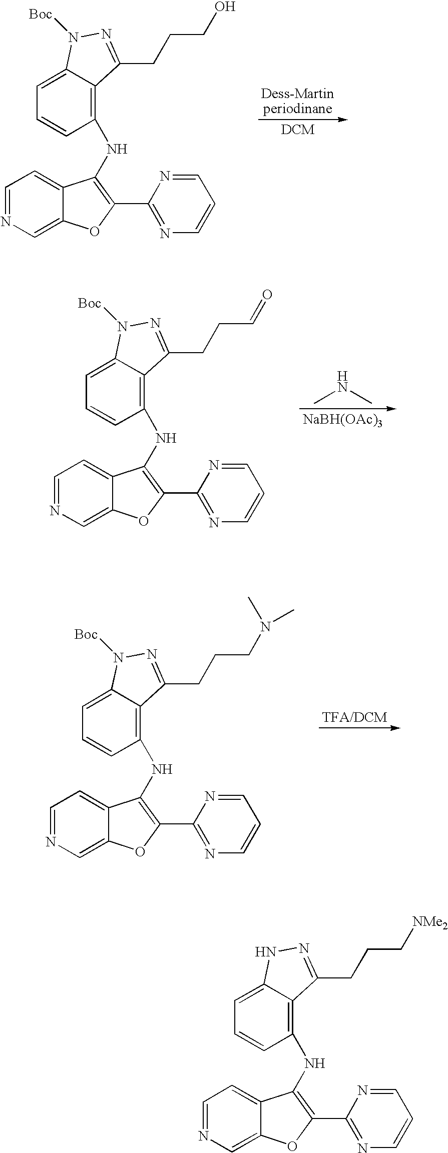

Step A: Preparation of tert-butyl 3-(3-oxopropyl)-4-(2-(pyrimidin-2-yl)furo[2,3-c]pyridin-3-ylamino-1H-indazole-1-carboxylate: Dess-Martin periodinane (0.157 g, 0.37 mmol) was added to a solution of tert-butyl 3-(3-hydroxypropyl)-4-(2-(pyrimidin-2-yl)furo[2,3-c]pyridin-3-ylamino)-1H-indazole-1-carboxylate (0.12 g, 0.25 mmol) in dichloromethane (10 mL). The reaction mixture was stirred at room temperature for 30 minutes. The reaction was quenched with water (10 mL), and the aqueous layer was extracted with dichloromethane (20 mL×3). The combined organics were dried, filtered and concentrated. The crude product was purified by flash column chromatography, eluting with hexanes/ethyl acetate (1:2), hexanes/ethyl acetate (1:4) to give the desired product (0.083 g, 70%). MS (APCI-pos) M+1=485.0.

Step B: Preparation of tert-butyl 3-(3-(dimethylamino)propyl)-4-(2-pyrimidin-2-yl)furo[2,3-c]pyridin-3-ylamino)-1H-indazole-1-carboxylate: 2.0 dimethylamine solution in THF (0.86 mL, 1.71 mmol) was added to a solution of tert-butyl 3-(3-oxopropyl)-4-(2-(pyrimidin-2-yl)furo[2,3-c]pyridin-3-ylamino)-1H-indazole-1-carboxylate (0.83 g, 0.171 mmol) in dichloromethane (10 mL). The reaction mixture was stirred for 10 minutes before sodium triacetoxyborohydride (0.036 g, 0.171 mmol) was added. The mixture was left at room temperature for 16 hours. The reaction was carefully quenched by MeOH and then concentrated. The residue was taken up with dichloromethane (20 mL) and water (20 mL), and the aqueous layer was extracted with dichloromethane (50 ml×3). The combined organics were dried, filtered and concentrated. The crude product was purified by flash column chromatography, eluting with ethyl acetate, dichloromethane/MeOH (20:1) to give the desired product (0.075 g, 85%). MS (APCI-pos) M+1=514.0.

Step C: Preparation of 3-(3-(dimethylamino)propyl)-N-(2-(pyrimidin-2-yl)furo[2,3-c]pyridin-3-yl)-1H-indazol-4-amine: TFA (2.0 mL) was added dropwise to a solution of tert-butyl 3-(3-(dimethylamino)propyl)-4-(2-(pyrimidin-2-yl)furo[2,3-c]pyridin-3-ylamino)-1H-indazole-1-carboxylate (0.088 g, 0.17 mmol) in dichloromethane (2.0 mL). The reaction mixture was stirred at room temperature for 2 hours. The crude mixture was concentrated, and the residue was treated with saturated aqueous NaHCO₃ (5.0 mL). The aqueous layer was extracted with dichloromethane (20 mL×3), and the combined organics were dried, filtered and concentrated. The crude product was purified by flash column chromatography, eluting with dichloromethane/MeOH (10:1), dichloromethane/MeOH/triethylamine (10:1:0.1) to give of the desired product (0.044 g, 62%). MS (APCI-pos) M+1=414.2.

### Example 19

### Preparation of 3-(4-(2-(pyrimidin-2-yl)furo[2,3-c]pyridin-3-ylamino)-1H-indazol-3-yl)propanoic acid

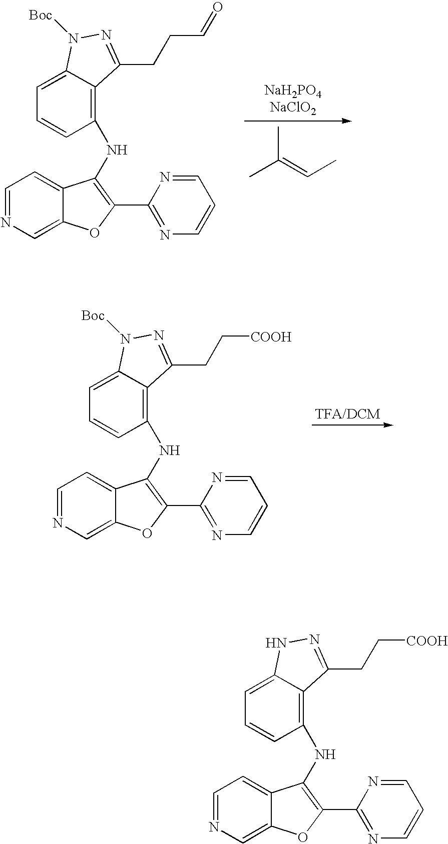

Step A: Preparation of 3-(1-(tert-butoxycarbonyl)-4-(2-(pyrimidin-2-yl)furo[2,3-c]pyridin-3-ylamino)-1H-indazol-3-yl)propanoic acid: 2-Methylbut-2-ene (2.0 M solution in THF, 0.64 mL, 1.29 mmol), sodium chlorite (0.07 g, 0.77 mmol), and NaH₂PO₄ (0.124 g, 1.03 mmol) as a solution in 2.0 mL of water was added to a solution of tert-butyl 3-(3-oxopropyl)-4-(2-(pyrimidin-2-yl)furo[2,3-c]pyridin-3-ylamino)-1H-indazole-1-carboxylate (0.125 g, 0.258 mmol) in t-BuOH (5.0 mL). The reaction mixture was stirred at room temperature for 48 hours. The crude mixture was concentrated, and the residue was diluted with water (10 mL). The pH was adjusted to about 5 with AcOH. The aqueous layer was extracted with dichloromethane (20 mL×3), and the combined organics were dried, filtered and concentrated. The crude product was purified by flash column chromatography, eluting with ethyl acetate to give the desired product (0.068 g, 53%). MS (APCI-pos) M+1=501.0.

Step B: Preparation of 3-(4-(2-(pyrimidin-2-yl)furo[2,3-c]pyridin-3-ylamino)-1H-indazol-3-yl)propanoic acid: TFA (2.0 mL) was added dropwise to a solution of 3-(1-(tert-butoxycarbonyl)-4-(2-(pyrimidin-2-yl)furo[2,3-c]pyridin-3-ylamino)-1H-indazol-3-yl)propanoic acid (0.068 g, 0.14 mmol) in dichloromethane (2.0 mL). The reaction mixture was stirred at room temperature for 3 hours. The crude mixture was concentrated and the residue was taken up in water (4.0 mL). The pH was adjusted to about 5 with saturated aqueous NaHCO₃. The resulting solids (0.028 g, 51%) were collected by filtration, washed with water (˜20 mL), dichloromethane and dried in vacuo. MS (APCI-pos) M+1=401.1.

### Example 20

### Preparation of 3-(3-aminopropyl)-N-(2-(pyrimidin-2-yl)furo[2,3-c]pyridin-3-yl)-1H-indazol-4-amine

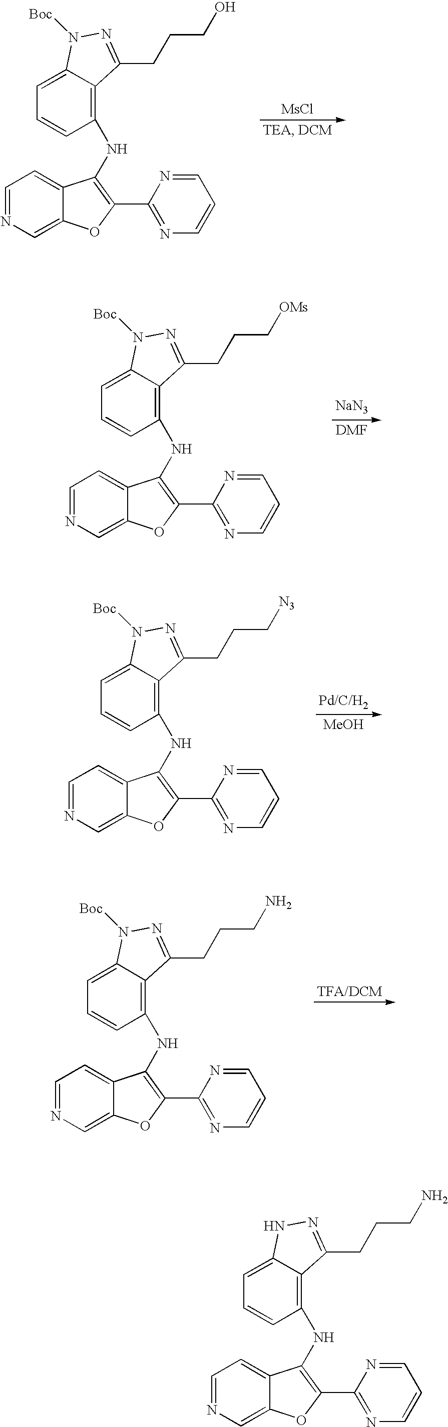

Step A: Preparation of tert-butyl 3-(3-(methylsulfonyloxy)propyl)-4-(2-(pyrimidin-2-yl)furo[2,3-c]pyridin-3-ylamino)-1H-indazole-1-carboxylate: Methane sulfonyl chloride (0.069 mL, 0.89 mmol) was added to a solution of tert-butyl 3-(3-hydroxypropyl)-4-(2-(pyrimidin-2-yl)furo[2,3-c]pyridin-3-ylamino)-1H-indazole-1-carboxylate (0.310 g, 0.64 mmol) and triethylamine (0.178 mL, 1.27 mmol) in dichloromethane (20 mL). The reaction mixture was stirred at room temperature for 3 hours. The crude mixture was concentrated and used directly in step B. MS (APCI-pos) M+1=656.0.

Step B: Preparation of tert-butyl 3-(3-azidopropyl)-4-(2-(pyrimidin-2-yl)furo[2,3-c]pyridin-3-ylamino)-1H-indazole-1-carboxylate: NaN₃ (0.043 g, 0.666 mmol) was added to a solution of tert-butyl 3-(3-(methylsulfonyloxy)propyl)-4-(2-(pyrimidin-2-yl)furo[2,3-c]pyridin-3-ylamino)-1H-indazole-1-carboxylate (0.188 g, 0.333 mmol) in DMF (10 mL). The reaction mixture was stirred at a temperature of 100° C. for 3 hours. The crude mixture was diluted with ethyl acetate (50 mL) and water (20 mL). The aqueous layer was extracted with ethyl acetate (50 mL×3), and the combined organics were dried, filtered and concentrated. The crude product was purified by flash column chromatography, eluting with hexanes/ethyl acetate (1:1), hexanes/ethyl acetate (1:2) to give the desired product (0.053 g, 25%) as a film. MS (APCI-pos) M+1=512.0.

Step C: Preparation of tert-butyl 3-(3-aminopropyl)-4-(2-(pyrimidin-2-yl)furo[2,3-c]pyridin-3-ylamino)-1H-indazole-1-carboxylate: 10% Pd/C (0.011 g, 0.010 mmol) was added to a solution of tert-butyl 3-(3-azidopropyl)-4-(2-(pyrimidin-2-yl)furo[2,3-c]pyridin-3-ylamino)-1H-indazole-1-carboxylate (0.053 g, 0.10 mmol) in MeOH (10 mL). The reaction mixture was purged with N₂ and hydrogenated with H₂ (14 psi) for 3 hours. The reaction mixture was filtered (GF/F paper) and the filtrate was concentrated to give the desired product (0.045 g, 89%). MS (APCI-pos) M+1=485.9.

Step D: Preparation of 3-(3-aminopropyl)-N-(2-(pyrimidin-2-yl)furo[2,3-c]pyridin-3-yl)-1H-indazol-4-amine: TFA (2.0 mL) was added dropwise to a solution of tert-butyl 3-(3-aminopropyl)-4-(2-(pyrimidin-2-yl)furo[2,3-c]pyridin-3-ylamino)-1H-indazole-1-carboxylate (0.088 g, 0.17 mmol) in dichloromethane (2.0 mL). The reaction mixture was stirred at room temperature for 2 hours. The crude mixture was concentrated, and the residue was treated with saturated aqueous NaHCO₃ (5.0 mL). The aqueous layer was extracted with dichloromethane (20 mL×3), and the combined organics were dried, filtered and concentrated. The crude product was purified by flash column chromatography, eluting with dichloromethane/MeOH (10:1), dichloromethane/MeOH/triethylamine (12:1:0.1), dichloromethane/MeOH/triethylamine (10:1:0.1) to give the desired product (0.032 g, 65%). MS (APCI-pos) M+1=386.2.

### Example 21

### Preparation of 3-(3-(methylamino)propyl)-N-(2-(pyrimidin-2-yl)furo[2,3-c]pyridin-3-yl)-1H-indazol-4-amine

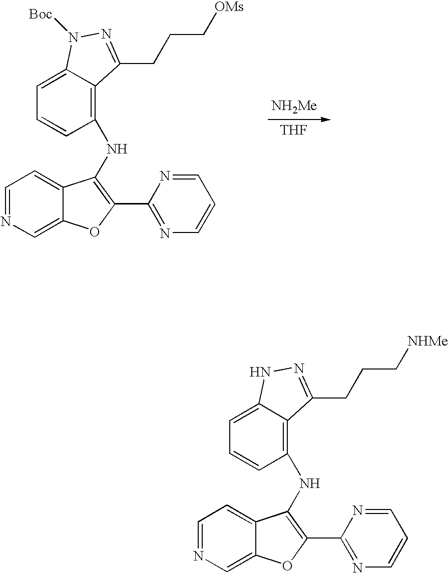

2.0 M methyl amine in THF (17.7 mL, 35.4 mmol) was added to a solution of tert-butyl 3-(3-(methylsulfonyloxy)propyl)-4-(2-(pyrimidin-2-yl)furo[2,3-c]pyridin-3-ylamino)-1H-indazole-1-carboxylate (0.100 g, 0.177 mmol) in THF (10 mL). The reaction mixture was stirred at room temperature for 16 hours. The crude product was concentrated and purified by flash column chromatography, eluting with dichloromethane/MeOH (10:1), dichloromethane/MeOH/triethylamine (10:1:0.1) to give the desired product (0.017 g, 24%). MS (APCI-pos) M+1=400.1.

### Example 22

### Preparation of 3,7-dichloro-N-(2-(pyrimidin-2-yl)furo[2,3-c]pyridin-3-yl)-1H-indazol-4-amine

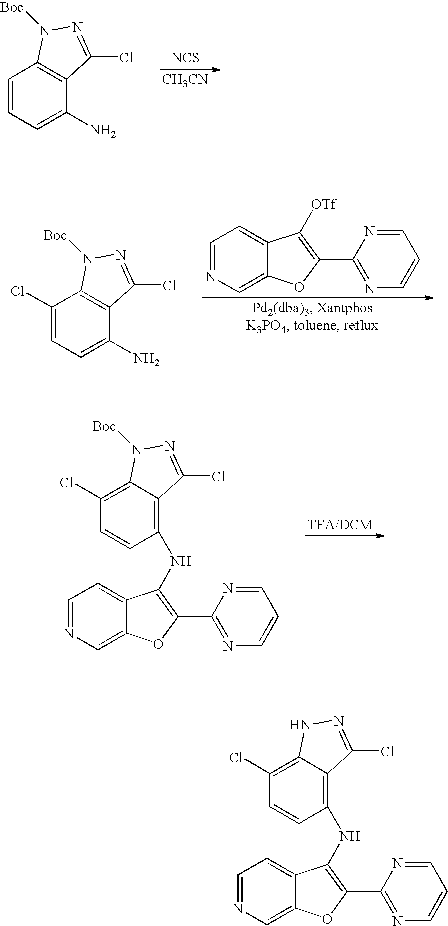

Step A: Preparation of tert-butyl 4-amino-3,7-dichloro-1H-indazole-1-carboxylate: N-chlorosuccinimide (0.183 g, 1.37 mmol) was added to a solution of tert-butyl 4-amino-3-chloro-1H-indazole-1-carboxylate (0.334 g, 1.25 mmol) in acetonitrile (20 mL). The reaction mixture was stirred at 60° C. for 16 hours. The crude mixture was concentrated and purified by flash column chromatography, eluting with hexanes/ethyl acetate (10:1) to give the desired product (0.127 g, 34%).

Step B: Preparation of tert-butyl 3,7-dichloro-4-(2-(pyrimidin-2-yl)furo[2,3-c]pyridin-3-ylamino)-1H-indazole-1-carboxylate: 2-(Pyrimidin-2-yl)furo[2,3-c]pyridin-3-yl trifluoromethanesulfonate (0.14 g, 0.40 mmol) and tert-butyl 4-amino-3,7-dichloro-1H-indazole-1-carboxylate (0.129 g, 0.43 mmol) were suspended in toluene (25 mL) and degassed with argon for 15 minutes. Xantphos (0.047 g, 0.081 mmol), Pd₂(dba)₃ (0.037 g, 0.040 mmol) and K₃PO₄ (0.189 g, 0.89 mmol) were added. The reaction mixture was degassed for another 15 minutes and then heated at reflux under argon overnight. The reaction mixture was filtered (GF/F paper), and the filtrate was purified by flash column chromatography, eluting with hexanes/ethyl acetate (1:1), ethyl acetate, to give the desired product (0.141 g, 70%). MS (APCI-pos) M+1=496.8, 498.8.

Step C: Preparation of 3,7-dichloro-N-(2-(pyrimidin-2-yl)furo[2,3-c]pyridin-3-yl)-1H-indazol-4-amine: TFA (2.0 mL) was added dropwise to a suspension of tert-butyl 3,7-dichloro-4-(2-(pyrimidin-2-yl)furo[2,3-c]pyridin-3-ylamino)-1H-indazole-1-carboxylate (0.141 g, 0.28 mmol) in dichloromethane (2.0 mL). The reaction mixture was stirred at room temperature for 3 hours. The crude mixture was concentrated, and the residue was neutralized to a pH of about 7 with saturated aqueous NaHCO₃ (5.0 mL). The resulting solids (0.062 g, 55%) were collected by filtration, washed sequentially with water (˜20 mL), ethyl acetate (˜20 mL) and dried in vacuo. MS (APCI-pos) M+1=397.4, 399.3.

### Example 23

### Preparation of 3-(7-chloro-4-(2-(pyrimidin-2-yl)furo[2,3-c]pyridin-3-ylamino)-1H-indazol-3-yl)propan-1-ol

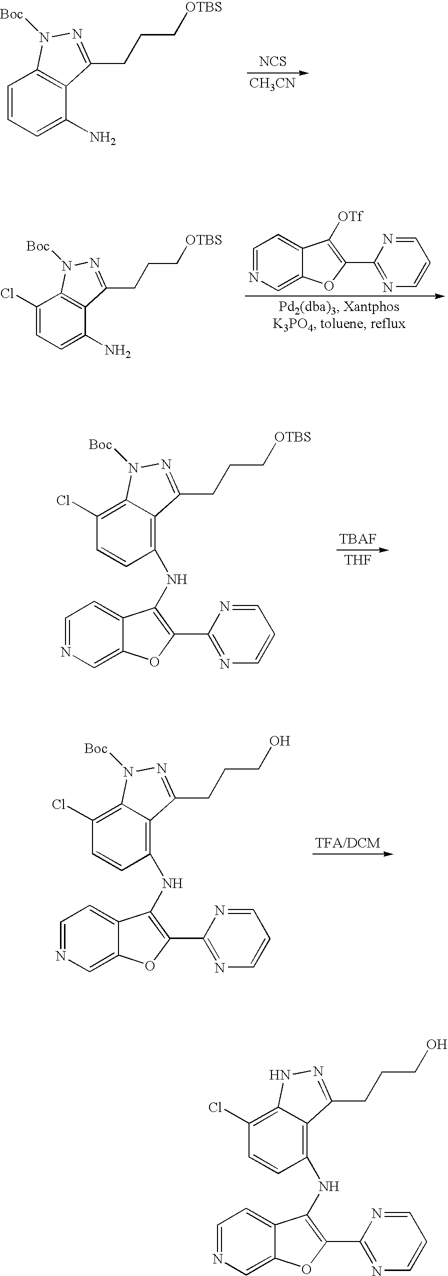

Step A: Preparation of tert-butyl 4-amino-3-(3-(tert-butyldimethylsilyloxy)propyl)-7-chloro-1H-indazole-1-carboxylate: N-chlorosuccinimide (0.057 g, 0.426 mmol) was added to a solution of tert-butyl 4-amino-3-(3-(tert-butyldimethylsilyloxy)propyl)-1H-indazole-1-carboxylate (0.157 g, 0.387 mmol) in acetonitrile (20 mL). The reaction mixture was stirred at 60° C. for 16 hours. The crude mixture was concentrated and purified by flash column chromatography, eluting with hexanes/ethyl acetate (10:1) to give the desired product (0.038 g, 22%). MS (APCI-pos) M+1=439.8, 441.8.

Step B: Preparation of tert-butyl 3-(3-(tert-butyldimethylsilyloxy)propyl)-7-chloro-4-(2-(pyrimidin-2-yl)furo[2,3-c]pyridin-3-ylamino)-1H-indazole-1-carboxylate: 2-(Pyrimidin-2-yl)furo[2,3-c]pyridin-3-yl trifluoromethanesulfonate (0.807 g, 2.34 mmol) and tert-butyl 4-amino-3-(3-(tert-butyldimethylsilyloxy)propyl)-7-chloro-1H-indazole-1-carboxylate (0.98 g, 2.23 mmol) were suspended in toluene (50 mL) and degassed with argon for 15 minutes. Xantphos (0.129 g, 0.22 mmol), Pd₂(dba)₃ (0.102 g, 0.11 mmol) and K₃PO₄ (0.756 g, 3.56 mmol) were added. The reaction mixture was degassed for another 15 minutes and then heated at reflux under argon overnight. The reaction mixture was filtered (GF/F paper), and the filtrate was purified by flash column chromatography, eluting with hexanes/ethyl acetate (1:1) to give the desired product (0.756 g, 53%). MS (APCI-pos) M+1=635.0, 637.0.

Step C: Preparation of tert-butyl 7-chloro-3-(3-hydroxypropyl)-4-(2-(pyrimidin-2-yl)furo[2,3-c]pyridin-3-ylamino)-1H-indazole-1-carboxylate: 1.0 M of tetra-butyl ammonium fluoride solution in THF (3.57 mL, 3.57 mmol) was added to a solution of tert-butyl 3-(3-(tert-butyldimethylsilyloxy)propyl)-7-chloro-4-(2-(pyrimidin-2-yl)furo[2,3-c]pyridin-3-ylamino)-1H-indazole-1-carboxylate (0.756 g, 1.1.9 mmol) in THF (10 mL). The reaction mixture was stirred at room temperature for 3 hours before quenching with water (10 mL). The aqueous layer was extracted with ethyl acetate (50 mL×3), and the combined organics were dried, filtered and concentrated. The crude product was purified by flash column chromatography, eluting with ethyl acetate, dichloromethane/MeOH (20:1), to give the desired product (0.288 g, 47%). MS (APCI-pos) M+1=521.0, 523.0.

Step D: Preparation of 3-(7-chloro-4-(2-(pyrimidin-2-yl)furo[2,3-c]pyridin-3-ylamino)-1H-indazol-3-yl)propan-1-ol: TFA (2.0 mL) was added dropwise to a solution of tert-butyl 7-chloro-3-(3-hydroxypropyl)-4-(2-(pyrimidin-2-yl)furo[2,3-c]pyridin-3-ylamino)-1H-indazole-1-carboxylate (0.050 g, 0.096 mmol) in dichloromethane (2.0 mL). The reaction mixture was stirred at room temperature for 3 hours. The crude mixture was concentrated. The residue was taken up in dichloromethane (4.0 mL) and treated with triethylamine (1 mL) for 30 minutes. The reaction mixture was concentrated, and the solids (0.022 g, 54%) were collected by filtration, washed with water (˜20 mL) and dried in vacuo. MS (APCI-pos) M+1=421.3, 423.2.

### Example 24

### Preparation of 3-(3-aminopropyl)-7-chloro-N-(2-(pyrimidin-2-yl)furo[2,3-c]pyridin-3-yl)-1H-indazol-4-amine

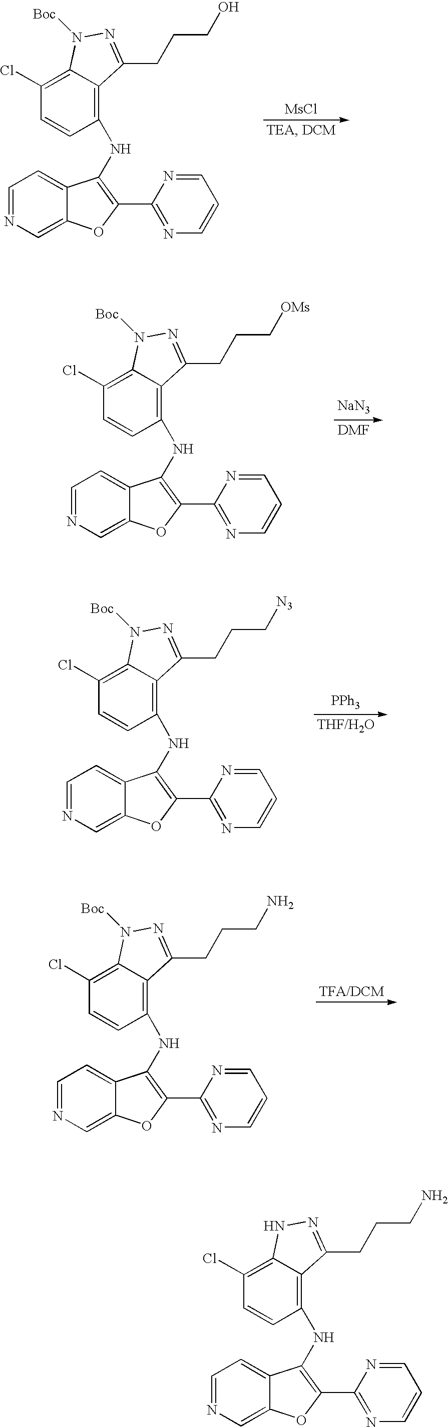

Step A: Preparation of tert-butyl 7-chloro-3-(3-(methylsulfonyloxy)propyl)-4-(2-(pyrimidin-2-yl)furo[2,3-c]pyridin-3-ylamino)-1H-indazole-1-carboxylate: Methanesulfonyl chloride (0.050 mL, 0.64 mmol) was added to a solution of tert-butyl 7-chloro-3-(3-hydroxypropyl)-4-(2-(pyrimidin-2-yl)furo[2,3-c]pyridin-3-ylamino)-1H-indazole-1-carboxylate (0.24 g, 0.46 mmol) and triethylamine (0.129 mL, 0.92 mmol) in dichloromethane (20 mL). The reaction mixture was stirred at room temperature for 3 hours. The crude mixture was concentrated and used directly in step B. MS (APCI-pos) M+1=598.9, 600.9.

Step B: Preparation of tert-butyl 3-(3-azidopropyl)-7-chloro-4-(2-(pyrimidin-2-yl)furo[2,3-c]pyridin-3-ylamino)-1H-indazole-1-carboxylate: NaN₃ (0.060 g, 0.92 mmol) was added to a solution of tert-butyl 7-chloro-3-(3-(methylsulfonyloxy)propyl)-4-(2-(pyrimidin-2-yl)furo[2,3-c]pyridin-3-ylamino)-1H-indazole-1-carboxylate (0.276 g, 0.46 mmol) in DMF (10 mL). The reaction mixture was stirred at a temperature of 100° C. for 3 hours. The crude mixture was diluted with ethyl acetate (50 mL) and water (20 mL). The aqueous layer was extracted with ethyl acetate (50 mL×3), and the combined organics were dried, filtered and concentrated. The crude product was purified by flash column chromatography, eluting with hexanes/ethyl acetate (1:1), hexanes/ethyl acetate (1:2) to give the desired product (0.18 g, 72%) as a film MS (APCI-pos) M+1=546.0, 548.0.

Step C: Preparation of tert-butyl 3-(3-aminopropyl)-7-chloro-4-(2-(pyrimidin-2-yl)furo[2,3-c]pyridin-3-ylamino)-1H-indazole-1-carboxylate: Triphenylphosphine (0.112 g, 0.429 mmol) was added to a solution of tert-butyl 3-(3-azidopropyl)-7-chloro-4-(2-(pyrimidin-2-yl)furo[2,3-c]pyridin-3-ylamino)-1H-indazole-1-carboxylate (0.180 g, 0.33 mmol) in THF/H₂O (9:1, 10 mL). The reaction mixture was heated at reflux for 2 hours. The crude product was concentrated and purified by flash column chromatography, eluting with hexanes/ethyl acetate (1:1), ethyl acetate to give the desired product (0.018 g, 11%) as a film MS (APCI-pos) M+1=519.9, 521.9.

Step D: Preparation of 3-(3-aminopropyl)-7-chloro-N-(2-(pyrimidin-2-yl)furo[2,3-c]pyridin-3-yl)-1H-indazol-4-amine: TFA (2.0 mL) was added dropwise to a solution of tert-butyl 3-(3-aminopropyl)-7-chloro-4-(2-(pyrimidin-2-yl)furo[2,3-c]pyridin-3-ylamino)-1H-indazole-1-carboxylate (0.019 g, 0.036 mmol) in dichloromethane (2.0 mL). The reaction mixture was stirred at room temperature for 2 hours. The crude mixture was concentrated, and the residue was treated with saturated aqueous NaHCO₃ (5.0 mL). The aqueous layer was extracted with dichloromethane (20 mL×3), and the combined organics were dried, filtered and concentrated. The crude product was purified by flash column chromatography, eluting with dichloromethane/MeOH (10:1), dichloromethane/MeOH/triethylamine (12:1:0.1), dichloromethane/MeOH/triethylamine (10:1:0.1) to give the desired product (0.008 g, 55%). MS (APCI-pos) M+1=420.1, 422.1.

### Example 25

### Preparation of 2-(5-bromopyrimidin-2-yl)-3-(tert-butyldiphenylsilyloxy)furo[2,3-c]pyridine

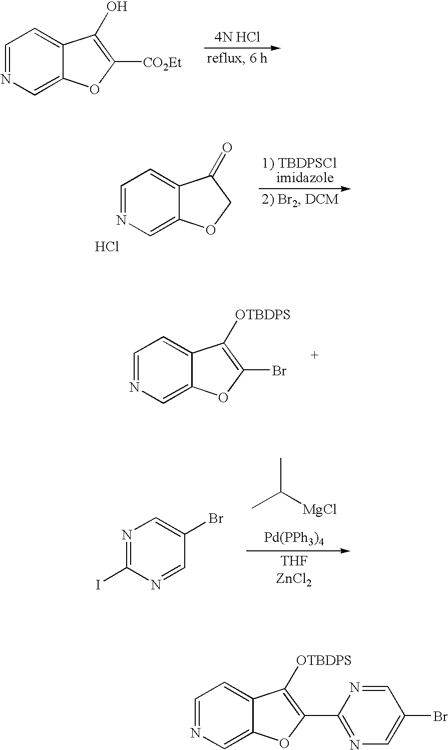

Step A: Preparation of furo[2,3-c]pyridin-3(2H)-one hydrochloride: Ethyl 3-hydroxyfuro[2,3-c]pyridine-2-carboxylate (2.0 g, 9.65 mmol) was suspended in 4 M HCl (10 mL) and heated at reflux for 6 hours. The reaction was cooled and concentrated to give a solid (1.60 g, 95%). The crude product was used directly in step B. MS (APCI-pos) M+1=136.4.

Step B: Preparation of 3-(tert-butyldiphenylsilyloxy)furo[2,3-c]pyridine: Imidazole (1.65 g, 24.2 mmol) and tert-butylchlorodiphenylsilane (3.71 mL, 14.5 mmol) were sequentially added to a suspension of furo[2,3-c]pyridin-3(2H)-one hydrochloride (1.60 g, 9.67 mmol) in dichloromethane (100 mL). The mixture was stirred at room temperature for 16 hours before quenching with water (50 mL). The aqueous layer was extracted with dichloromethane (100 mL×3). The combined organic were dried, filtered and concentrated. The crude product was purified by flash column chromatography, eluting with hexanes/ethyl acetate (9:1) to give the desired product (3.39 g, 94%). ¹HNMR (400 MHz, CDCl₃) δ 8.7 (s, 1H), 8.4 (d, J=5.6 Hz, 1H), 7.7 (m, 4H), 7.5 (m, 31-1), 7.4 (m, 4H), 6.8 (s, 1H), 1.2 (s, 9H) ppm. MS (APCI-pos) M+1=374.3.

Step C: Preparation of 2-bromo-3-(tert-butyldiphenylsilyloxy)furo[2,3-c]pyridine: Br₂ (1.67 g, 10.4 mmol) in solution with CHCl₃ (5.0 mL) was added to a solution of 3-(tert-butyldiphenylsilyloxy)furo[2,3-c]pyridine (1.30 g, 3.48 mmol) in CHCl₃ (20 mL). The mixture was stirred at room temperature for 1 hour before quenching with saturated aqueous Na₂S₂O₃ and saturated aqueous NaHCO₃. The aqueous layer was extracted with dichloromethane (100 mL×3). The combined organics were dried, filtered and concentrated (bath temperature of about 20° C.). The crude product was purified by flash column chromatography, eluting with dichloromethane, dichloromethane/ethyl acetate (9:1) to give the desired product (1.42 g, 90%). ¹HNMR (400 MHz, CDCl₃) δ 8.6 (s, 1H), 8.0 (d, J=5.6 Hz, 1H), 7.7 (m, 4H), 7.4 (m, 6H), 6.7 (d, J=5.6 Hz, 1H), 1.2 (s, 9H) ppm. MS (APCI-pos) M+1=452.3, 454.2.

Step D: Preparation of 2-(5-bromopyrimidin-2-yl)-3-(tert-butyldiphenylsilyloxy)furo[2,3-c]pyridine: Isopropyl MgCl (“i-PrMgCl”, 2.0 M in THF, 1.12 mL, 2.23 mmol) was added slowly via a syringe to a flame dried 50 mL round bottom flask (“RBF”) containing 2-bromo-3-(tent-butyldiphenylsilyloxy)furo[2,3-c]pyridine (0.674 g, 1.49 mmol) in cold (−10° C.) THF (20 mL). The reaction mixture was stirred at a temperature of 10° C. for 1 hour. ZnCl₂ (0.5 M solution in THF, 4.47 mL, 2.23 mmol) was added. The cold bath was removed, and the reaction mixture was stirred at room temperature for 15 minutes. Pd(PPh₃)₄ (0.172 g, 0.149 mmol), 5.0 mL anhydrous THF and 5-bromo-2-iodopyrimidine (0.637 g, 2.23 mmol) were charged under argon in another 50 mL flame dried RBF. The aryl zinc solution was added to this via a cannula. The reaction mixture was left at room temperature under argon overnight. The reaction mixture was concentrated, and the residue was diluted with water (20 mL) and ethyl acetate (50 mL). The aqueous layer was extracted with ethyl acetate (50 mL×3). The combined organics were dried, filtered and concentrated. The crude product was purified by flash column chromatography, eluting with hexanes/ethyl acetate (4:1), hexanes/ethyl acetate (2:1) to give the desired product (0.62 g, 79%). ¹H NMR (400 MHz, CDCl₃) δ 8.9 (s, 1H), 8.7 (s, 2H), 8.1 (d, J=5.6 Hz, 1H), 7.8 (m, 4H), 7.4-7.3 (m, 6H), 6.9 (d, J=5.6 Hz, 1H), 1.2 (s, 9H) ppm. MS (APCI-pos) M+1=530.3, 532.3.

### Example 26

### Preparation of 3-(2-(3-(7-chloro-1H-indazol-4-ylamino)furo[2,3-c]pyridin-2-yl)pyrimidin-5-yl)propan-1-ol

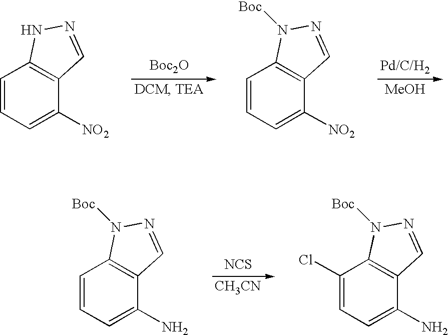

Step A: Preparation of tert-butyl 4-nitro-1H-indazole-1-carboxylate: Triethylamine (5.3 mL, 38 mmol) was added to a suspension of 4-nitro-1H-indazole (5.2 g, 32 mmol) in dichloromethane (100 mL), followed by Boc₂O (7.7 g, 35 mmol) addition. The reaction was stirred at room temperature for 16 hours and then quenched with water (50 mL). The aqueous layer was extracted with dichloromethane (50 mL×3), and the combined organics were dried, filtered and concentrated. The crude product was purified by flash column chromatography, eluting with hexanes/ethyl acetate (9:1) to give the desired product (8.1 g, 97%).

Step B: Preparation of tert-butyl 4-amino-1H-indazole-1-carboxylate: 10% Pd/C (0.9 g) was added to a solution of tert-butyl 4-nitro-1H-indazole-1-carboxylate (8.1 g, 30.8 mmol) in a mixture of MeOH/ethyl acetate (1:4, 100 mL). The reaction mixture was purged with N₂ and hydrogenated with H₂ (30 psi) for 16 hours. The reaction mixture was filtered (GF/F paper), and the filtrate was concentrated. The crude product was purified by flash column chromatography, eluting with hexanes/ethyl acetate (4:1), hexanes/ethyl acetate (1:1) to give the desired product (6.0 g, 84%). MS (APCI-pos) M+1=233.7.

Step C: Preparation of tert-butyl 4-amino-7-chloro-1H-indazole-1-carboxylate: N-chlorosuccinimide (1.19 g, 1.37 mmol) was added to a solution of tert-butyl 4-amino-1H-indazole-1-carboxylate (1.90 g, 8.14 mmol) in acetonitrile (50 mL). The reaction mixture was stirred at a temperature of 60° C. for 16 hours. The crude mixture was concentrated and purified by flash column chromatography, eluting with hexanes/ethyl acetate (4:1), hexanes/ethyl acetate (3:2) to give the desired product (0.731 g, 34%). ¹H NMR (400 MHz, CDCL₃) δ=8.10 (s, 1H), 7.29 (d, J=8.0 Hz, 1H), 6.46 (d, J=8.8 Hz, 1H), 4.18 (br s, 2H), 1.71 (s, 9H).

### Example 27

### Preparation of 7-chloro-N-(2-(5-methoxypyrimidin-2-yl)furo[2,3-c]pyridin-3-yl)-1H-indazol-4-amine

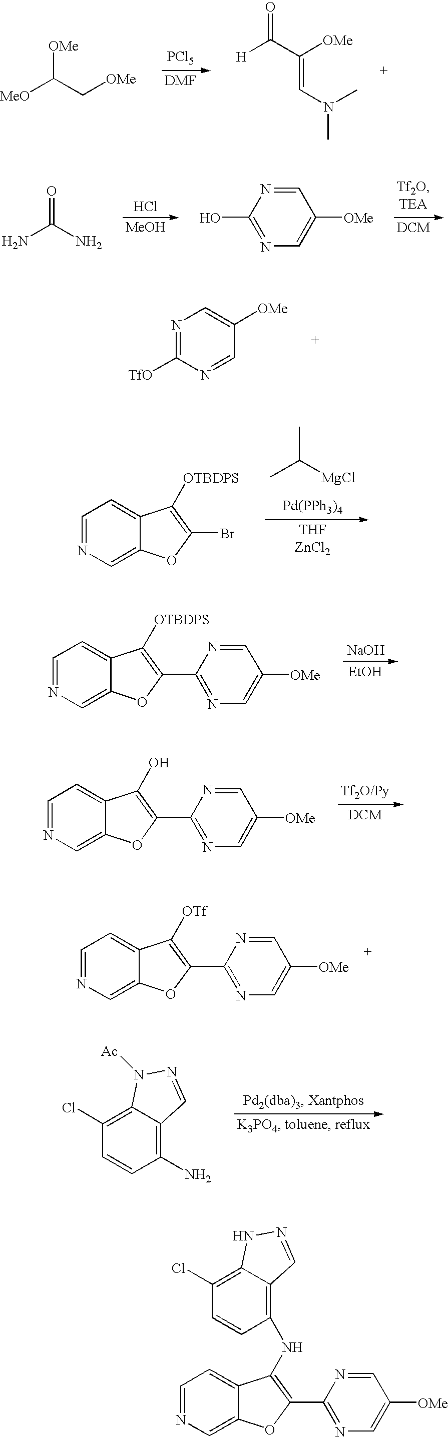

Step A: Preparation of (Z)-3-(dimethylamino)-2-methoxyacrylaldehyde: The reaction was carried out in a 3-neck flask (500 mL) equipped with an internal thermometer. PCl₅ (64.4 g, 294 mmol) was added in small portions (˜5 g) to a cold (0° C.) solution of 1,1,2-trimethoxyethane (36 g, 294 mmol) while keeping the internal temperature below 30° C. The mixture was heated to 60° C. for 75 minutes and then placed in an ice bath. DMF (66 mL, 852 mmol) was added via a dropping funnel while maintaining the internal temperature below a temperature of 10° C. The mixture was stirred at room temperature for 40 hours, and MeOH (100 mL) was added dropwise via a dropping funnel while keeping the internal temperature below 10° C. The solution was transferred to an addition funnel and was added dropwise to a 30% solution of sodium methoxide (403 mL, 2.17 mol) in MeOH while maintaining the internal temperature below 20° C. The mixture was heated at reflux for 4 hours and then concentrated. The residue was taken up in water (500 mL) and extracted with dichloromethane (500 mL×3). The combined organics were dried, filtered and concentrated to give the desired product (19 g, 25%). MS (APCI-pos) M+1=130.0.

Step B: Preparation of 5-methoxypyrimidin-2-ol: (Z)-3-(dimethylamino)-2-methoxyacrylaldehyde (17.1 g, 66 mmol) and urea (15.9 g, 265 mmol) were suspended in MeOH (100 mL) and treated with a concentrated HCl solution (12 mL). The mixture was heated at reflux for 16 hours and then concentrated. The crude product was purified by flash column chromatography, eluting with dichloromethane/MeOH (20:1), dichloromethane/MeOH (10:1) to give the desired product (5.6 g, 34%).

Step C: Preparation of 5-methoxypyrimidin-2-yl trifluoromethanesulfonate: A suspension of 5-methoxypyrimidin-2-ol (1.02 g, 0.09 mmol) and triethylamine (2.26 mL, 16.2 mmol) in dichloromethane (50 mL) was stirred at a temperature of 0° C. for 10 minutes. Tf₂O (2.72 mL, 16.2 mmol) was added and stirring was continued for 30 minutes. The mixture was quenched with water (50 mL), and the aqueous layer was extracted with dichloromethane (50 mL×3). The combined organics were dried, filtered and concentrated. The crude product was purified by flash column chromatography, eluting with hexanes/ethyl acetate (20:1) to give the desired product (0.5 g, 24%).

Step D: Preparation of 3-(tert-butyldiphenylsilyloxy)-2-(5-methoxypyrimidin-2-yl)furo[2,3-c]pyridine: i-PrMgCl (2.0 M in THF, 1.19 mL, 2.39 mmol) was added slowly via a syringe to a flame dried 50 mL RBF containing 2-bromo-3-(tert-butyldiphenylsilyloxy)furo[2,3-c]pyridine (0.90 g, 1.59 mmol) in cold (−10° C.) THF (20 mL). The reaction was stirred at a temperature of −10° C. for 1 hour. ZnCl₂ (0.5 M solution in THF, 4.8 mL, 2.39 mmol) was added. The cold bath was removed, and the reaction mixture was stirred at room temperature for 15 minutes. Pd(PPh₃)₄ (0.184 g, 0.159 mmol), 5.0 mL anhydrous THF and 5-methoxypyrimidin-2-yl trifluoromethanesulfonate (0.431 g, 1.67 mmol) was charged under argon in another 50 mL flame dried round bottom flask. The aryl zinc solution was added via a cannula. The reaction mixture was left at room temperature under argon overnight. The reaction mixture was concentrated, and the residue was diluted with water (20 mL) and ethyl acetate (50 mL). The aqueous layer was extracted with ethyl acetate (50 mL×3). The combined organics were dried, filtered and concentrated. The crude product was purified by flash column chromatography, eluting with hexanes/ethyl acetate (4:1), hexanes/ethyl acetate (1:1) to give the desired product (0.40 g, 52%). MS (APCI-pos) M+1=482.4.

Step E: Preparation of 2-(5-methoxypyrimidin-2-yl)furo[2,3-c]pyridin-3-ol: 4 N NaOH (0.25 mL, 1.0 mmol) was added to a solution of 3-(tert-butyldiphenylsilyloxy)-2-(5-methoxypyrimidin-2-yl)furo[2,3-c]pyridine (0.400 g, 0.83 mmol) in EtOH (10 mL). The mixture was stirred at room temperature for 2 hours. The reaction mixture was acidified with AcOH (0.3 mL) and then concentrated. The crude product was used directly in Step F. MS (APCI-pos) M+1=244.3.

Step F: Preparation of 2-(5-methoxypyrimidin-2-yl)furo[2,3-c]pyridin-3-yl trifluoromethanesulfonate: A suspension of 2-(5-methoxypyrimidin-2-yl)furo[2,3-c]pyridin-3-ol (0.202 g, 0.83 mmol) and pyridine (0.087 mL, 1.08 mmol) in dichloromethane (50 mL) was stirred at a temperature of 0° C. for 10 minutes. Tf₂O (0.168 mL, 1.0 mmol) was added, and the mixture was stirred for 1 hour. The mixture was quenched with water (50 mL), and the aqueous layer was extracted with dichloromethane (50 mL×3). The combined organics were dried, filtered and concentrated. The crude product was purified by flash column chromatography; eluting with hexanes/ethyl acetate (2:1) to give the desired product (0.16 g, 51%). MS (APCI-pos) M+1=375.9.

Step G: Preparation of 7-chloro-N-(2-(5-methoxypyrimidin-2-yl)furo[2,3-c]pyridin-3-yl)-1H-indazol-4-amine: 245-Methoxypyrimidin-2-yl)furo[2,3-c]pyridin-3-yl trifluoromethanesulfonate (0.160 g, 0.426 mmol) and 1-(4-amino-7-chloro-1H-indazol-1-yl)ethanone (0.107 g, 0.512 mmol) were suspended in toluene (20 mL) and degassed with argon for 15 minutes. Xantphos (0.049 g, 0.085 mmol), Pd₂(dba)₃ (0.039 g, 0.043 mmol) and K₃PO₄ (0.199 g, 0.938 mmol) were added. The reaction mixture was degassed for another 15 minutes and then heated at reflux under argon overnight. The reaction mixture was filtered (GF/F paper), and the filtrate was purified by flash column chromatography, eluting with hexanes/ethyl acetate (1:1), hexanes/ethyl acetate (1:4) to give the desired product (0.004 g, 2%). MS (APCI-pos) M+1=393.4, 395.3.

### Example 28

### Preparation of 3-(2-(3-(7-chloro-1H-indazol-4-ylamino)furo[2,3-c]pyridin-2-yl)pyrimidin-5-yl)propan-1-ol

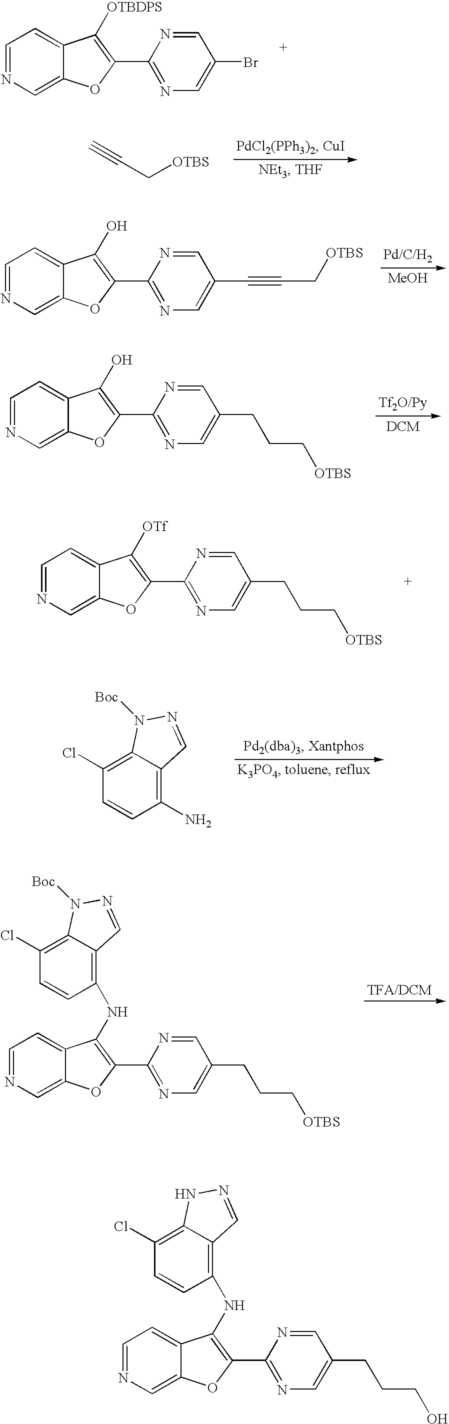

Step A: Preparation of 2-(5-(3-(tert-butyldimethylsilyloxy)prop-1-ynyl)pyrimidin-2-yl)furo[2,3-c]pyridin-3-ol: PdCl₂(PPh₃)₂ (0.074 g, 0.11 mmol) and CuI (0.038 g, 0.20 mmol) were added to a solution of 2-(5-bromopyrimidin-2-yl)-3-(tert-butyldiphenylsilyloxy)furo[2,3-c]pyridine (0.70 g, 1.32 mmol), tert-butyldimethyl(prop-2-ynyloxy)silane (0.67 g, 3.96 mmol) and triethylamine (4 mL) in THF (20 mL). The mixture was purged with argon for 15 minutes and then stirred at room temperature under argon for 16 hours. The reaction mixture was filtered (GF/F paper), and the filtrate was purified by flash column chromatography, eluting with hexanes/ethyl acetate (2:1) to give the desired product (0.20 g, 40%). MS (APCI-pos) M+1=382.3.

Step B: Preparation of 2-(5-(3-(tert-butyldimethylsilyloxy)propyl)pyrimidin-2-yl)furo[2,3-c]pyridin-3-ol: 10% Pd/C (0.042 g, 0.039 mmol) was added to a solution of 2-(5-(3-(tert-butyldimethylsilyloxy)prop-1-ynyl)pyrimidin-2-yl)furo[2,3-c]pyridin-3-ol (0.15 g, 0.39 mmol) in MeOH (20 mL). The reaction mixture was purged with N₂ and hydrogenated with H₂ (14 psi) for 1 hour. The reaction mixture was filtered (GF/F paper), and the filtrate was concentrated. The crude product was purified by flash column chromatography, eluting with hexanes/ethyl acetate (2:1) to give the desired product (0.096 g, 63%). MS (APCI-pos) M+1=386.4.

Step C: Preparation of 2-(5-(3-(tert-butyldimethylsilyloxy)propyl)pyrimidin-2-yl)furo[2,3-c]pyridin-3-yl trifluoromethanesulfonate: A suspension of 2-(5-(3-(tert-butyldimethylsilyloxy)propyl)pyrimidin-2-yl)furo[2,3-c]pyridin-3-ol (0.096 g, 0.25 mmol), pyridine (0.026 mL, 0.32 mmol) in dichloromethane (20 mL) was stirred at a temperature of 0° C. for 10 minutes. Tf₂O (0.050 mL, 0.30 mmol) was added and stirring was continued for 1 hour. The mixture was quenched with water (20 mL), and the aqueous layer was extracted with dichloromethane (50 mL×3). The combined organics were dried, filtered and concentrated. The crude product was purified by flash column chromatography, eluting with hexanes/ethyl acetate (2:1) to give the desired product (0.073 g, 57%). MS (APCI-pos) M+1=518.1.

Step D: Preparation of tert-butyl 4-(2-(5-(3-(tert-butyldimethylsilyloxy)propyl)pyrimidin-2-yl)furo[2,3-c]pyridin-3-ylamino)-7-chloro-1H-indazole-1-carboxylate: 2-(5-(3-(Tert-butyldimethyl silyloxy)propyl)pyrimidin-2-yl)furo[2,3-c]pyridin-3-yl trifluoromethanesulfonate (0.073 g, 0.141 mmol) and tert-butyl 4-amino-7-chloro-1H-indazole-1-carboxylate (0.041 g, 0.155 mmol) were suspended in toluene (20 mL) and degassed with argon for 15 minutes. Xantphos (0.025 g, 0.042 mmol), Pd₂(dba)₃ (0.019 g, 0.021 mmol) and K₃PO₄ (0.048 g, 0.226 mmol) were added. The reaction mixture was degassed for another 15 minutes and then heated at reflux under argon overnight. The reaction mixture was filtered (GF/F paper), and the filtrate was purified by flash column chromatography, eluting with hexanes/ethyl acetate (2:1), hexanes/ethyl acetate (1:4) to give the desired product (0.070 g, 78%). MS (APCI-pos) M+1=634.9, 636.9.

Step E: Preparation of 3-(2-(3-(7-chloro-1H-indazol-4-ylamino)furo[2,3-c]pyridin-2-yl)pyrimidin-5-yl)propan-1-ol: TFA (2.0 mL) was added dropwise to a solution of tert-butyl 4-(2-(5-(3-(tert-butyldimethylsilyloxy)propyl)pyrimidin-2-yl)furo[2,3-c]pyridin-3-ylamino)-7-chloro-1H-indazole-1-carboxylate (0.070 g, 0.11 mmol) in dichloromethane (2.0 mL). The reaction mixture was stirred at room temperature for 2 hours. The crude mixture was concentrated. The residue was taken up in dichloromethane (4.0 mL) and treated with triethylamine (1 mL) for 30 minutes. The crude mixture was concentrated, and the crude product was purified by flash column chromatography, eluting with hexanes/ethyl acetate (2:1), hexanes/ethyl acetate (1:4) to give the desired product (0.020 g, 35%). MS (APCI-pos) M+1=421.4, 423.4.

### Example 29

### Preparation of N-(2-(pyrimidin-2-yl)furo[2,3-c]pyridin-3-yl)-1H-benzo[d]imidazol-4-amine

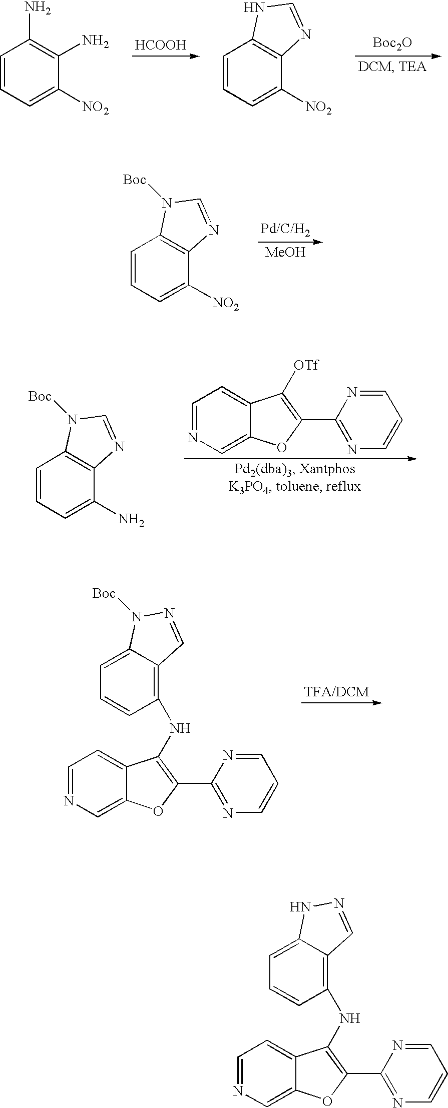

Step A: Preparation of 4-nitro-1H-benzo[d]imidazole: A solution of 3-nitrobenzene-1,2-diamine (1.0 g, 6.5 mmol) in formic acid (10 mL) was heated at reflux for 16 hours. The reaction mixture was cooled to room temperature and concentrated. The resulting solids were suspended in water and treated with saturated aqueous NaHCO₃ until the pH was about 7 to about 8. The solids were collected by filtration and dried in vacuo to give the desired product (1.0 g, 94%). MS (APCI-neg) M−1=162.2.

Step B: Preparation of tert-butyl 4-nitro-1H-benzo[d]imidazole-1-carboxylate: Triethylamine (1.03 mL, 7.36 mmol) was added to a suspension of 4-nitro-1H-benzo[d]imidazole (1.0 g, 6.13 mmol) in dichloromethane (50 mL), followed by addition of Boc₂O (1.61 g, 7.36 mmol). The reaction was stirred at room temperature for 16 hours and then quenched with water (20 mL). The aqueous layer was extracted with dichloromethane (50 mL×3), and the combined organics were dried, filtered and concentrated. The crude product was purified by flash column chromatography, eluting with hexanes/ethyl acetate (4:1) to give the desired product (1.50 g, 93%).

Step C: Preparation of tert-butyl 4-amino-1H-benzo[d]imidazole-1-carboxylate: 10% Pd/C (0.013 g, 0.012 mmol) was added to a solution of tert-butyl 4-nitro-1H-benzo[d]imidazole-1-carboxylate (1.50 g, 5.70 mmol) in MeOH (20 mL). The reaction mixture was purged with N₂ and hydrogenated with H₂ (30 psi) for 2 hours. The reaction mixture was filtered (GF/F paper) and the filtrate was concentrated to give the desired product (1.32 g, 99%). MS (APCI-pos) M+1=233.7.

Step D: Preparation of tert-butyl 4-(2-(pyrimidin-2-yl)furo[2,3-c]pyridin-3-ylamino)-1H-benzo[d]imidazole-1-carboxylate: 2-(Pyrimidin-2-yl)furo[2,3-c]pyridin-3-yl trifluoromethanesulfonate (0.10 g, 0.29 mmol) and tert-butyl 4-amino-1H-benzo[d]imidazole-1-carboxylate (0.081 g, 0.348 mmol) were suspended in toluene (5 mL) and degassed with argon for 15 minutes. Xantphos (0.033 g, 0.058 mmol), Pd₂(dba)₃ (0.027 g, 0.029 mmol) and K₃PO₄ (0.135 g, 0.64 mmol) were added. The reaction mixture was degassed for another 15 minutes and then heated at reflux under argon overnight. The reaction mixture was filtered (GF/F paper), and the filtrate was purified by flash column chromatography, eluting with hexanes/ethyl acetate (1:1), ethyl acetate to give the desired product (0.074 g, 60%). MS (APCI-pos) M+1=429.0.

Step E: Preparation of N-(2-(pyrimidin-2-yl)furo[2,3-c]pyridin-3-yl)-1H-benzo[d]imidazol-4-amine: TFA (2.0 mL) was added dropwise to a suspension of tert-butyl 4-(2-(pyrimidin-2-yl)furo[2,3-c]pyridin-3-ylamino)-1H-benzo[d]imidazole-1-carboxylate (0.074 g, 0.17 mmol) in dichloromethane (2.0 mL). The reaction mixture was stirred at room temperature for 1 hour. The crude mixture was concentrated, and the residue was neutralized to a pH of about 7 with saturated aqueous NaHCO₃ (5.0 mL). The resulting solids (0.040 g, 71%) were collected by filtration, washed sequentially with water (˜20 mL), ethyl acetate (˜20 mL) and dried in vacuo. MS (APCI-pos) M+1=329.3.

### Example 30

### Preparation of N-(2-(pyrimidin-2-yl)furo[2,3-c]pyridin-3-yl)-1H-indazol-6-amine

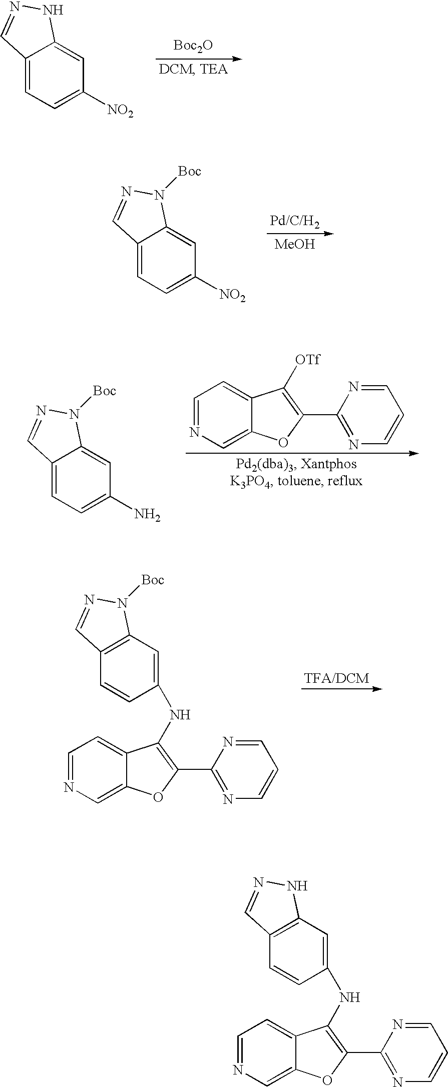

Step A: Preparation of tert-butyl 6-nitro-1H-indazole-1-carboxylate: Triethylamine (1.71 mL, 12.3 mmol) was added to a suspension of 6-nitro-1H-indazole (2.0 g, 12.3 mmol) in dichloromethane (50 mL), followed by addition of Boc₂O (2.62 g, 12.0 mmol). The reaction was stirred at room temperature for 16 hours and then quenched with water (20 mL). The aqueous layer was extracted with dichloromethane (50 mL×3), and the combined organics were dried, filtered and concentrated. The crude product was purified by flash column chromatography, eluting with hexanes/ethyl acetate (9:1) to give the desired product (2.91 g, 90%).

Step B: Preparation of tert-butyl 6-amino-1H-indazole-1-carboxylate: 10% Pd/C (0.355 g, 0.334 mmol) was added to a solution of tert-butyl 6-nitro-1H-indazole-1-carboxylate (0.88 g, 3.34 mmol) in MeOH (20 mL). The reaction mixture was purged with N₂ and hydrogenated with H₂ (30 psi) for 2 hours. The reaction mixture was filtered (GF/F paper), and the filtrate was concentrated to give the desired product (0.76 g, 98%). MS (APCI-pos) M+1=233.8.

Step C: Preparation of tert-butyl 6-(2-(pyrimidin-2-yl)furo[2,3-c]pyridin-3-ylamino)-1H-indazole-1-carboxylate: 2-(Pyrimidin-2-yl)furo[2,3-c]pyridin-3-yl trifluoromethanesulfonate (0.082 g, 0.24 mmol) and tert-butyl 6-amino-1H-indazole-1-carboxylate (0.083 g, 0.354 mmol) were suspended in toluene (5 mL) and degassed with argon for 15 minutes. Xantphos (0.027 g, 0.047 mmol), Pd₂(dba)₃ (0.043 g, 0.047 mmol) and K₃PO₄ (0.110 g, 0.52 mmol) were added. The reaction mixture was degassed for another 15 minutes and then heated at reflux under argon overnight. The reaction mixture was filtered (GF/F paper), and the filtrate was purified by flash column chromatography, eluting with hexanes/ethyl acetate (1:1), hexanes/ethyl acetate (2:3), to give the desired product (0.018 g, 18%). MS (APCI-pos) M+1=429.0.

Step D: Preparation of N-(2-(pyrimidin-2-yl)furo[2,3-c]pyridin-3-yl)-1H-indazol-6-amine: TFA (2.0 mL) was added dropwise to a suspension of tert-butyl 6-(2-(pyrimidin-2-yl)furo[2,3-c]pyridin-3-ylamino)-1H-indazole-1-carboxylate (0.018 g, 0.034 mmol) in dichloromethane (2.0 mL). The reaction mixture was stirred at room temperature for 1 hour. The crude mixture was concentrated, and the residue was neutralized to a pH of about 7 with saturated aqueous NaHCO₃ (5.0 mL). The aqueous layer was extracted with dichloromethane (20 mL×3), and the combined organics were dried, filtered and concentrated. The crude product was purified by flash column chromatography, eluting with hexanes/ethyl acetate (1:4), ethyl acetate to give the desired product (0.005 g, 45%). MS (APCI-pos) M+1=329.4.

### Example 31

### Preparation of 7-chloro-3-ethyl-N-(2-(pyrimidin-2-yl)furo[2,3-c]pyridin-3-yl)-1H-indazol-4-amine

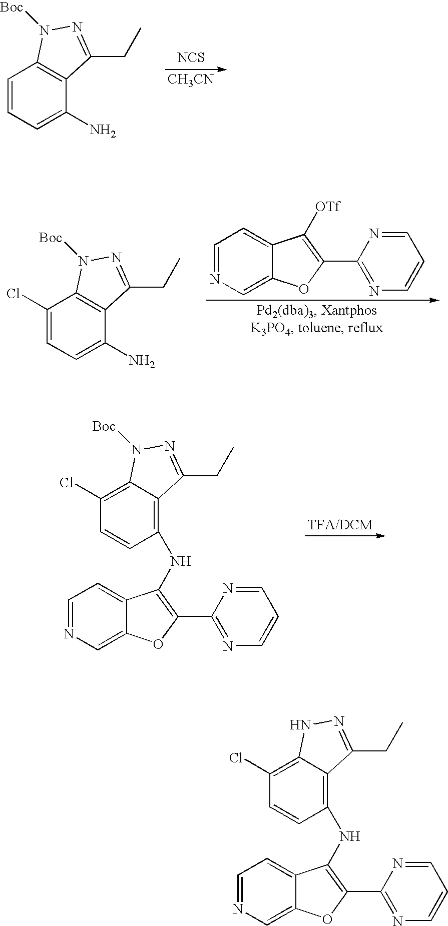

Step A: Preparation of tert-butyl 4-amino-7-chloro-3-ethyl-1H-indazole-1-carboxylate: N-chlorosuccinimide (190.1 mg, 1.424 mmol) was added to a solution of tert-butyl 4-amino-3-ethyl-1H-indazole-1-carboxylate (310 mg, 1.186 mmol) in acetonitrile (25 mL). The reaction mixture was stirred at a temperature of 60° C. for 16 hours. The crude mixture was concentrated and purified by flash column chromatography, eluting with hexanes/ethyl acetate (10:1) to give the desired product (125.2 mg, 36%). MS (APCI-pos) M+1=295.7, 297.7.

Step B: Preparation of tert-butyl 7-chloro-3-ethyl-4-(2-(pyrimidin-2-yl)furo[2,3-c]pyridin-3-ylamino)-1H-indazole-1-carboxylate: 2-(Pyrimidin-2-yl)furo[2,3-c]pyridin-3-yl trifluoromethanesulfonate (145.9 mg, 0.4226 mmol) and tert-butyl 4-amino-7-chloro-3-ethyl-1H-indazole-1-carboxylate (125 mg, 0.4226 mmol) were suspended in toluene (5 mL) and degassed with argon for 15 minutes. Xantphos (12.23 mg, 0.02113 mmol), Pd₂(dba)₃ (19.35 mg, 0.02113 mmol) and K₃PO₄ (134.6 mg, 0.6340 mmol) was added. The reaction mixture was degassed for another 15 minutes and then heated at reflux under argon overnight. The reaction mixture was filtered (GF/F paper), and the filtrate was purified by flash column chromatography, eluting with 3% MeOH/dichloromethane to give the desired product (148.2 mg, 71.4%). MS (APCI-neg) M−1=489.1, 491.1.

Step C: Preparation of 7-chloro-3-ethyl-N-(2-(pyrimidin-2-yl)furo[2,3-c]pyridin-3-yl)-1H-indazol-4-amine: TFA (2.0 mL) was added dropwise to a suspension of tert-butyl 7-chloro-3-ethyl-4-(2-(pyrimidin-2-yl)furo[2,3-c]pyridin-3-ylamino)-1H-indazole-1-carboxylate (148 mg, 0.301 mmol) in dichloromethane (2.0 mL). The reaction mixture was stirred at room temperature for 1 hour. The crude mixture was concentrated and purified by flash column chromatography, eluting with 5% MeOH/dichloromethane to give the desired product (0.0849 g, 72%). MS (APCI-pos) M+1=391.3, 393.3.

### Example 32

### Preparation of 1-(4-amino-6-chloro-1H-indazol-1-yl)ethanone

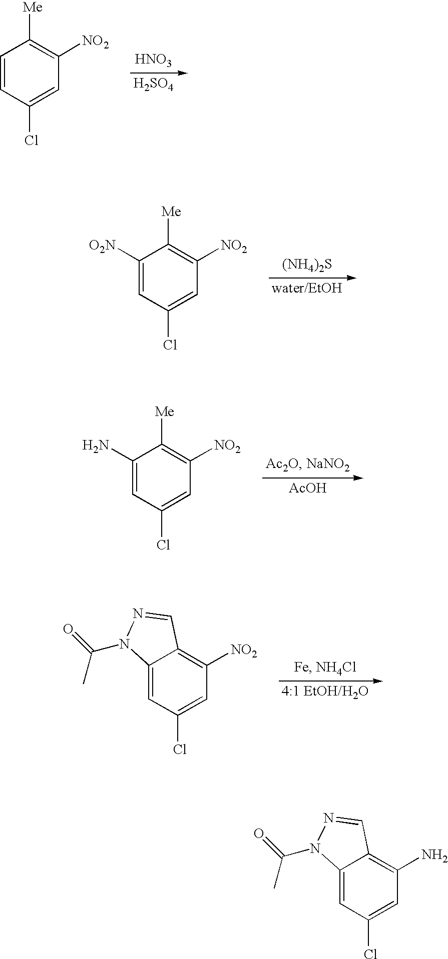

Step A: Preparation of 5-chloro-2-methyl-1,3-dinitrobenzene: A solution of 4-chloro-1-methyl-2-nitrobenzene (20.0 g, 116.6 mmol) in 60 mL concentrated sulfuric acid was cooled to a temperature of 0° C., and nitric acid (26.23 mL, 582.8 mmol) was added dropwise via an addition funnel maintaining the temperature below 60° C. The reaction was heated to a temperature of 90° C. for two hours and then cooled to room temperature. Water (1.0 L) was added, and the precipitate was collected by filtration and dried under high vacuum overnight. Purification by column chromatography with 50:1 hexanes/ethyl acetate provided the desired product (5.5 g, 21.8%). MS (APCI-neg) M+1=215.9, 217.9.

Step B: Preparation of 5-chloro-2-methyl-3-nitroaniline: 5-chloro-2-methyl-1,3-dinitrobenzene (2.14 g, 9.88 mmol) was taken up in 100 mL EtOH. A 50% aqueous solution of ammonium sulfide (6.75 mL, 49.4 mmol) was added, and the reaction was heated to a temperature of 50° C. for two hours. The reaction was cooled to room temperature and partitioned between ethyl acetate and water. The organic layer was separated and dried over Na₂SO₄, concentrated, and purified by silica gel chromatography (4:1 hexanes/ethyl acetate) to provide the title compound (0.039 g, 58.0%) as a solid.

Step C: Preparation of 1-(6-chloro-4-nitro-1H-indazol-1-yl)ethanone: The title compound was prepared as described in Example 7, Step C, substituting 5-chloro-2-methyl-3-nitroaniline for 2,6-dimethyl-3-nitroaniline to give the title compound as a solid. MS (APCI-neg) M+1=238.9, 240.9.

Step D: Preparation of 1-(4-amino-6-chloro-1H-indazol-1-yl)ethanone: 1-(6-Chloro-4-nitro-1H-indazol-1-yl)ethanone (0.0163 g, 0.0680 mmol), Fe (0) (0.0380 g, 0.680 mmol), and NH₄Cl (0.00182 g, 0.0340 mmol), were taken up in EtOH (1 mL) and water (0.25 mL) and heated to a temperature of 78° C. for 1 hour. The reaction was concentrated, taken up in dichloromethane to form a slurry, filtered through celite, and concentrated. Purification by silica gel chromatography (dichloromethane) provided the title compound (0.0055 g, 38.6%) as a solid. ¹H NMR (400 MHz, CDCl₃) δ 8.01 (s, 1H), 7.85 (s, 1H), 6.56 (s, 1H), 4.26 (br s, 2H), 2.76 (s, 3H).

### Example 33

### Preparation of N-(6-chloro-1H-indazol-4-yl)-2-(pyrimidin-2-yl)furo[2,3-c]pyridin-3-amine

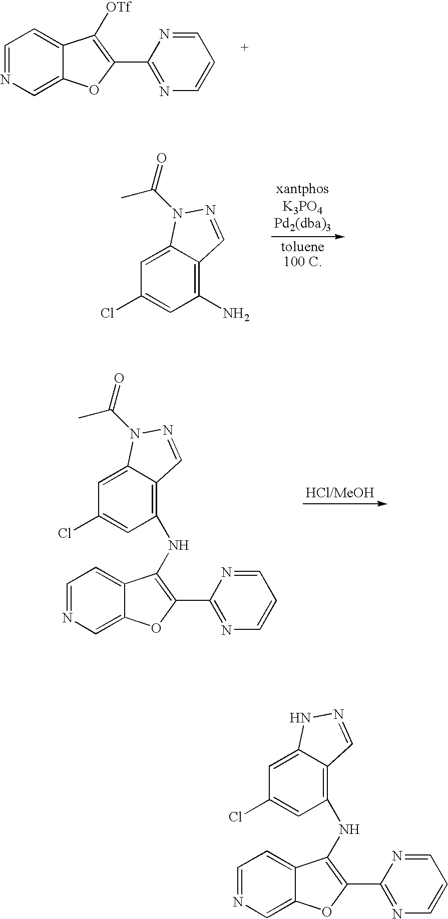

Step A: Preparation of 1-(6-chloro-4-(2-(pyrimidin-2-yl)furo[2,3-c]pyridin-3-ylamino)-1H-indazol-1-yl)ethanone: The title compound was prepared as described in Example 15, Step D, substituting 1-(4-amino-6-chloro-1H-indazol-1-yl)ethanone for tert-butyl 4-amino-3-chloro-1H-indazole-1-carboxylate to give the title compound as a solid. MS (APCI-pos) M+1=405.2, 407.2.

Step B: Preparation of N-(6-chloro-1H-indazol-4-yl)-2-(pyrimidin-2-yl)furo[2,3-c]pyridin-3-amine: 1-(6-Chloro-4-(2-(pyrimidin-2-yl)furo[2,3-c]pyridin-3-ylamino)-1H-indazol-1-yl)ethanone (0.003 g, 0.0074 mmol) was taken up in MeOH (1 mL), and 1.0 N HCl (0.037 mL, 0.037 mmol) was added. The reaction was heated to a temperature of 60° C. The reaction was partitioned between ethyl acetate and saturated aqueous NaHCO₃. The organic layer was separated and dried over Na₂SO₄, concentrated, and purified by silica gel chromatography (2% to 10% MeOH/dichloromethane) to provide the title compound (0.0018 g, 67.0%) as a solid. MS (APCI-pos) M+1=363.4, 365.4.

### Example 34

### Preparation of 1-(4-amino-6-methyl-1H-indazol-1-yl)ethanone

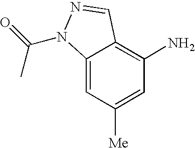

The compound was prepared as described in Example 32 substituting 1,4-dimethyl-2-nitrobenzene for 4-chloro-1-methyl-2-nitrobenzene to give the title compound as a solid. MS (APCI-pos) M+1=190.0.

### Example 35

### Preparation of N-(6-methyl-1H-indazol-4-yl)-2-(pyrimidin-2-yl)furo[2,3-c]pyridin-3-amine

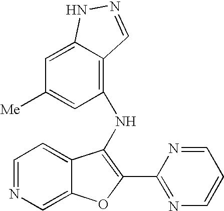

The compound was prepared as described in Example 33 substituting 1-(4-amino-6-methyl-1H-indazol-1-yl)ethanone for 1-(4-amino-6-chloro-1H-indazol-1-yl)ethanone to give the title compound as a solid. MS (APCI-pos) M+1=343.4.

### Example 36

### Preparation of 3-(7-chloro-1H-indazol-4-ylamino)-N-(pyrimidin-2-yl)furo[2,3-c]pyridine-2-carboxamide

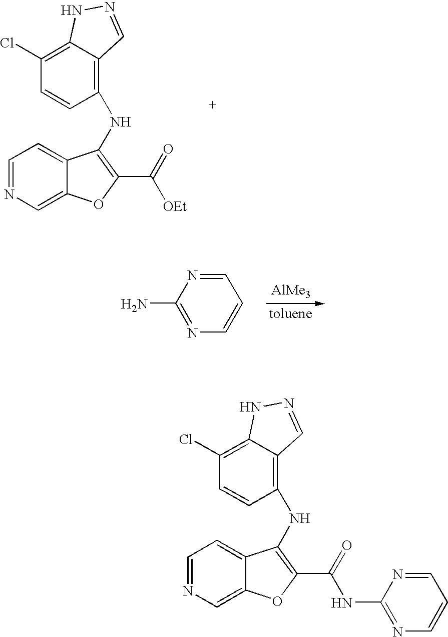

Pyrimidin-2-amine (0.0800 g, 0.841 mmol) was taken up in toluene (2 mL) and cooled to a temperature of 0° C. Trimethylaluminum (0.420 mL, 0.841 mmol) was added dropwise and stirred at a temperature of 0° C. for ten minutes. The mixture was warmed to room temperature. Ethyl 3-(7-chloro-1H-indazol-4-ylamino)furo[2,3-c]pyridine-2-carboxylate (0.060 g, 0.168 mmol) was dissolved in toluene (2.0 mL), added to the reaction via a syringe, and then heated to a temperature of 100° C. for 3 hours. The reaction was cooled to room temperature, quenched with water, and filtered through celite. Ethyl acetate was added, and the organic layer was separated and dried over Na₂SO₄, concentrated, and purified by silica gel chromatography (3% MeOH/dichloromethane) to provide the title compound (0.031 g, 46.0%) as a solid. MS (APCI-pos) M+1=406.2, 408.1.

### Example 37

### Preparation of 3-(7-chloro-1H-indazol-4-ylamino)-N-isopropylfuro[2,3-c]pyridine-2-carboxamide

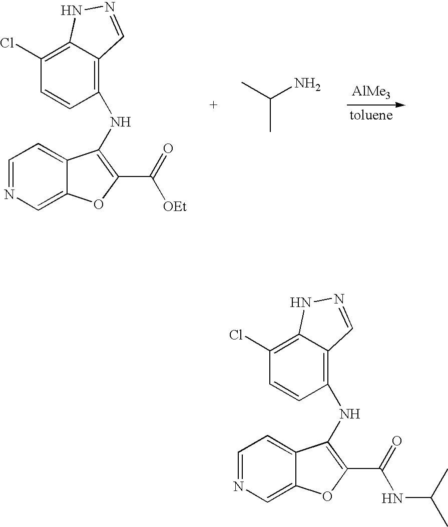

The compound was prepared as described in Example 36 substituting propan-2-amine for pyrimidin-2-amine to give the title compound as a solid. MS (APCI-pos) M+1=-370.2, 372.2.

### Example 38

### Preparation of 2-iodo-4,6-dimethylpyrimidine

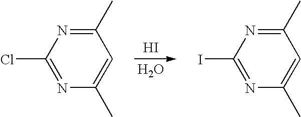

A 57% solution of hydriodic acid in water (13.9 mL, 105.2 mmol) was added to 2-chloro-4,6-dimethylpyrimidine (3.0 g, 21.04 mmol) and stirred at room temperature for three days. The reaction was neutralized with solid K₂CO₃ and decolorized with 10% KHSO₃. The solution was filtered through celite and then stored at a temperature of 5° C. overnight. The resulting solid was collected and dried under high vacuum providing the title compound (0.4 g, 8.3%). MS (APCI-pos) M+1=235.1.

### Example 39

### Preparation of 3-(tert-butyldiphenylsilyloxy)-2-(4,6-dimethylpyrimidin-2-yl)furo[2,3-c]pyridine

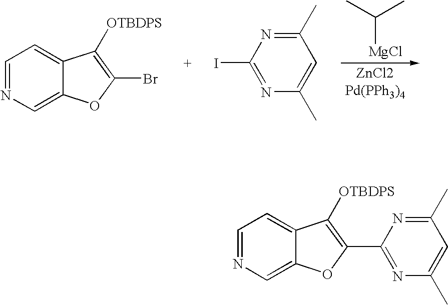

The compound was prepared as described in Example 27, Step D, substituting 2-iodo-4,6-dimethylpyrimidine for 5-methoxypyrimidin-2-yl trifluoromethanesulfonate to give the title compound as a solid. MS (APCI-pos) M+1=480.5.

### Example 40

### Preparation of 2-(4,6-dimethylpyrimidin-2-yl)furo[2,3-c]pyridin-3-yl trifluoromethanesulfonate

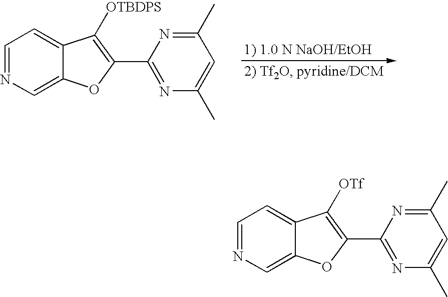

The compound was prepared as described in Example 27, Steps E and F, substituting 3-(tert-butyldiphenylsilyloxy)-2-(4,6-dimethylpyrimidin-2-yl)furo[2,3-c]pyridine for 3-(tert-butyldiphenylsilyloxy)-2-(5-methoxypyrimidin-2-yl)furo[2,3-c]pyridine. MS (APCI-pos) M+1=374.0.

### Example 41

### Preparation of 2-(4,6-dimethylpyrimidin-2-yl)-N-(1H-indazol-4-yl)furo[2,3-c]pyridin-3-amine

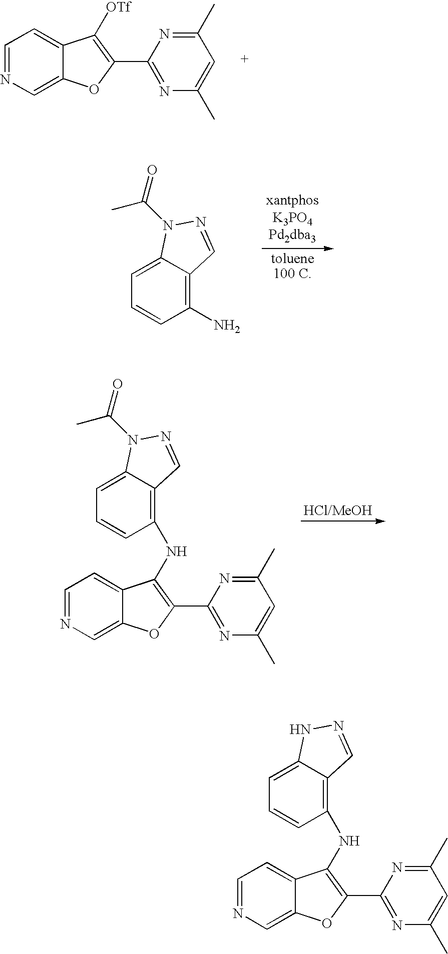

Step A: Preparation of 1-(4-(2-(4,6-dimethylpyrimidin-2-yl)furo[2,3-c]pyridin-3-ylamino)-1H-indazol-1-yl)ethanone: The compound was prepared as described in Example 15, Step D substituting 2-(4,6-dimethylpyrimidin-2-yl)furo[2,3-c]pyridin-3-yl trifluoromethanesulfonate for 2-(pyrimidin-2-yl)furo[2,3-c]pyridin-3-yl trifluoromethanesulfonate and 1-(4-amino-1H-indazol-1-yl)ethanone for tert-butyl 4-amino-3-chloro-1H-indazole-1-carboxylate. MS (APCI-pos) M+1=399.2.

Step B: Preparation of 2-(4,6-dimethylpyrimidin-2-yl)-N-(1H-indazol-4-yl)furo[2,3-c]pyridin-3-amine: 1-(4-(2-(4,6-Dimethylpyrimidin-2-yl)furo[2,3-c]pyridin-3-ylamino)-1H-indazol-1-yl)ethanone (0.0041 g, 0.010 mmol) was taken up in MeOH (1 mL), and 1.0 N HCl (0.11 mL, 0.011 mmol) was added. The reaction was heated to a temperature of 60° C. The reaction was partitioned between ethyl acetate and saturated aqueous NaHCO₃. The organic layer was separated and dried over Na₂SO₄, concentrated, and purified by silica gel chromatography (2% to 3% MeOH/dichloromethane) to provide the title compound (0.0017 g, 46.0%) as a solid. MS (APCI-pos) M+1=357.4.

### Example 42

### Preparation of 7-chloro-N-(furo[2,3-c]pyridin-3-yl)-1H-indazol-4-amine

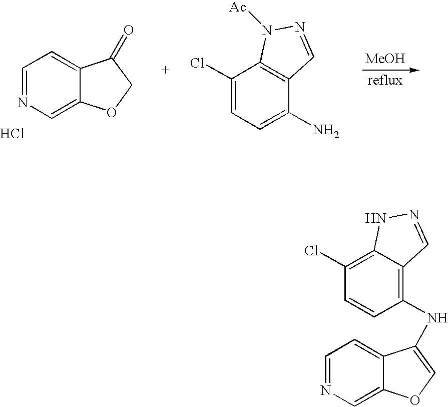

Furo[2,3-c]pyridin-3(2H)-one hydrochloride (0.30 g, 1.75 mmol) and 1-(4-amino-1H-indazol-1-yl)ethanone (0.367 g, 1.75 mmol) were suspended in MeOH (25 mL), and the mixture was heated at reflux for 40 hours. The reaction mixture was concentrated, and the residue was treated with saturated aqueous NaHCO₃ The resulting solids (0.262 g, 53%) were collected by filtration, washed with water (˜20 mL), dichloromethane and dried in vacuo. MS (APCI-pos) M+1=285.3, 287.2.

### Example 43

### Preparation of 7-chloro-2-(pyrimidin-2-yl)furo[2,3-c]pyridin-3-yl trifluoromethanesulfonate

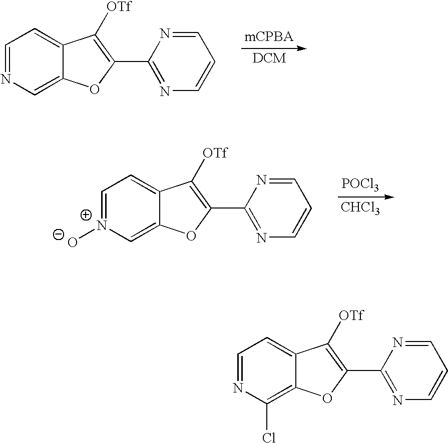

Step A: Preparation of 2-(pyrimidin-2-yl)furo[2,3-c]pyridin-3-yl trifluoromethanesulfonate-N-oxide: 3-Chlorobenzoperoxoic acid (1.07 g, 70% wt, 4.34 mmol) was added to a solution of 2-(pyrimidin-2-yl)furo[2,3-c]pyridin-3-yl trifluoromethanesulfonate (1.00 g, 2.90 mmol) in dichloromethane, and the mixture was left at room temperature for 16 hours. The crude mixture was concentrated and purified by silica gel chromatography, eluting with ethyl acetate, ethyl acetate/MeOH (20:1) to provide the title compound (0.964 g, 92%) as a yellow solid. MS (APCI-pos) M+1=362.0.

Step B: Preparation of 7-chloro-2-(pyrimidin-2-yl)furo[2,3-c]pyridin-3-yl trifluoromethanesulfonate: POCl₃ (1.47 mL, 16.0 mmol) was added to a cold (0° C.) solution of 2-(pyrimidin-2-yl)furo[2,3-c]pyridin-3-yl trifluoromethanesulfonate-N-oxide (0.964 g, 2.67 mmol) in CHCl₃ (20 mL). The cold bath was removed, and the mixture was refluxed for 16 hours. The reaction mixture was cooled and then concentrated. The crude product was purified by silica gel chromatography, eluting with hexanes/ethyl acetate (8:1) to provide the title compound (0.548 g, 54%) as a white solid. ¹H NMR (400 MHz, CDCl₃) δ 9.0 (d, J=4.7 Hz, 2H), 8.4 (d, J=5.6 Hz, 1H), 7.6 (d, J=5.6 Hz, 1H), 9.0 (t, J=4.7 Hz, 1H).

### Example 44

### Preparation of 7-chloro-N-(7-chloro-2-(pyrimidin-2-yl)furo[2,3-c]pyridin-3-yl)-1H-indazol-4-amine

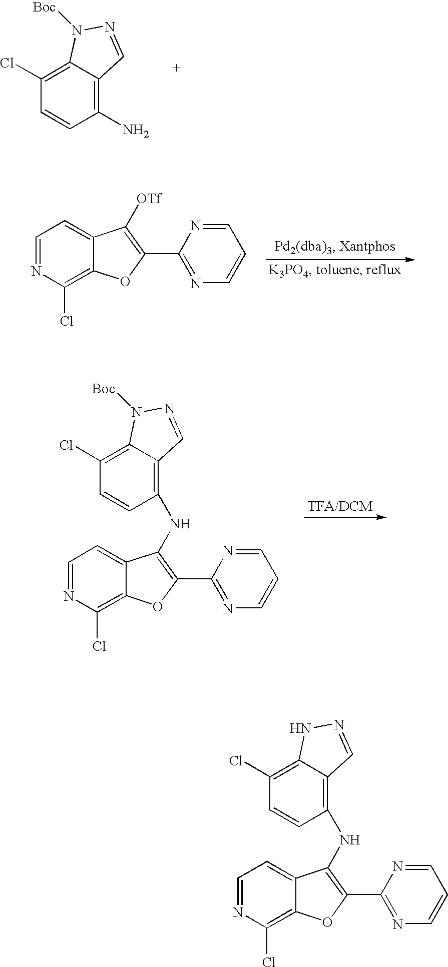

Step A: Preparation of tert-butyl 7-chloro-4-(7-chloro-2-(pyrimidin-2-yl)furo[2,3-c]pyridin-3-ylamino)-1H-indazole-1-carboxylate: Tert-butyl 7-chloro-4-(7-chloro-2-(pyrimidin-2-yl)furo[2,3-c]pyridin-3-ylamino)-1H-indazole-1-carboxylate was prepared according to the procedure outlined in Example 15, step D substituting tert-butyl 4-amino-7-chloro-1H-indazole-1-carboxylate for tert-butyl 4-amino-3-chloro-1H-indazole-1-carboxylate and 7-chloro-2-(pyrimidin-2-yl)furo[2,3-c]pyridin-3-yl trifluoromethanesulfonate for 2-(pyrimidin-2-yl)furo[2,3-c]pyridin-3-yl trifluoromethanesulfonate. MS (APCI-pos) M+1=496.9, 498.9.

Step B: Preparation of 7-Chloro-N-(7-chloro-2-(pyrimidin-2-yl)furo[2,3-c]pyridin-3-yl)-1H-indazol-4-amine: 7-Chloro-N-(7-chloro-2-(pyrimidin-2-yl)furo[2,3-c]pyridin-3-yl)-1H-indazol-4-amine was prepared according to the procedure outlined in Example 15, step E substituting tert-butyl 7-chloro-4-(7-chloro-2-(pyrimidin-2-yl)furo[2,3-c]pyridin-3-ylamino)-1H-indazole-1-carboxylate for tert-butyl 3-chloro-4-(2-(pyrimidin-2-yl)furo[2,3-c]pyridin-3-ylamino)-1H-indazole-1-carboxylate. MS (APCI-pos) M+1=397.3, 399.3.

### Example 45

### Preparation of 1-tert-butyl 3-methyl 4-amino-1H-indazole-1,3-dicarboxylate

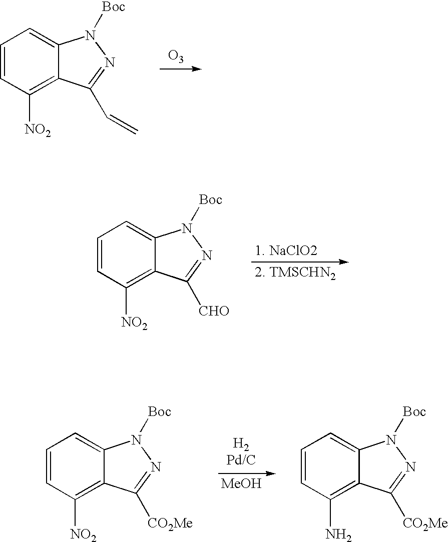

Step A: Preparation of tert-butyl 4-nitro-3-vinyl-1H-indazole-1-carboxylate: Ozone was bubbled through a solution of tert-butyl 4-nitro-3-vinyl-1H-indazole-1-carboxylate (3.0 g, 10.4 mmol) in CH₂Cl₂ (50 mL) at a temperature of −78° C. until the reaction mixture turned blue (approximately 45 minutes). PS-triphenylphosphine (7.1 g, 15 mmol based on 2.16 mmol/g loading) was added to the reaction, and the reaction was warmed to ambient temperature. The resin was filtered off, rinsing with CH₂Cl₂ (2×) and MeOH (2×). The filtrate was purified by silica gel chromatography (eluting with 5% ethyl acetate/hexanes to 40% ethyl acetate/hexanes, gradient) to afford tert-butyl 3-formyl-4-nitro-1H-indazole-1-carboxylate as a solid (642 mg, 21%). ¹H NMR (400 MHz, CDCl₃) δ 10.38 (s, 1H), 8.63 (d, J=8.5 Hz, 1H), 8.00 (d, J=7.1 Hz, 1H), 7.73 (m, 1H), 1.77 (s, 9H).

Steps B and C: Preparation of 1-tert-butyl 3-methyl 4-nitro-1H-indazole-1,3-dicarboxylate: A slurry of tert-butyl 3-formyl-4-nitro-1H-indazole-1-carboxylate (194 mg, 0.666 mmol) and 2.0 M solution of 2-methyl-2-butene in THF (3.33 mL, 6.66 mmol) in t-BuOH (6 mL) was treated with a mixture of NaH₂PO₄.H₂O (479 mg, 3.47 mmol) and sodium chlorite (tech. grade, 234 mg, 2.07 mmol) in water (2 mL) at room temperature. The reaction was stirred at ambient temperature for 4 hours, and the volatiles were removed via rotovap. The residue was taken up in CH₂Cl₂ and water, and the mixture neutralized with glacial AcOH (1 mL). The organic layer was collected and concentrated to afford crude 1-(tert-butoxycarbonyl)-4-nitro-1H-indazole-3-carboxylic acid. The crude acid was taken up in 4:1 THF:MeOH (10 mL), and 2.0 M solution of TMS-diazomethane in hexanes (0.5 mL, 1.0 mmol) was added. After 30 minutes, the volatiles were removed via rotovap, and the crude residue was purified by silica gel chromatography (eluting with 20% ethyl acetate/hexanes) to afford 1-tert-butyl 3-methyl 4-nitro-1H-indazole-1,3-dicarboxylate as a solid (149 mg, 70% over 2 steps). ¹HNMR (400 MHz, CDCl₃) δ 8.61 (d, J=8.6 Hz, 1H), 8.06 (d, J=7.8 Hz, 1H), 7.69 (m, 1H), 4.01 (s, 3H), 1.72 (s, 9H). MS (APCI-neg) M−1=320.9.

Step D: Preparation of 1-tert-butyl 3-methyl 4-amino-1H-indazole-1,3-dicarboxylate: A solution of 1-tert-butyl 3-methyl 4-nitro-1H-indazole-1,3-dicarboxylate (149 mg, 0.46 mmol) in MeOH (20 mL) was treated with 10% Pd/C (ca. 100 mg) and hydrogenated in a Parr shaker (40 psi H₂) for 2 hours. The reaction was filtered through GF/F paper, and the filtrate was purified by silica gel chromatography (eluting with 20% ethyl acetate/hexanes) to afford 1-tert-butyl 3-methyl 4-amino-1H-indazole-1,3-dicarboxylate (77 mg, 57%) as an oil. ¹H NMR (400 MHz, CDCl₃) δ 7.46 (d, J=8.7 Hz, 1H), 7.30 (t, J=8.2 Hz, 1H), 6.51 (d, J=7.9 Hz, 1H), 5.81 (br s, 2H), 4.03 (s, 3H), 1.73 (s, 9H). MS (APCI-neg) M-Boc−1=190.1.

### Example 46

### Preparation of methyl 4-(2-(pyrimidin-2-yl)furo[2,3-c]pyridin-3-ylamino)-1H-indazole-3-carboxylate

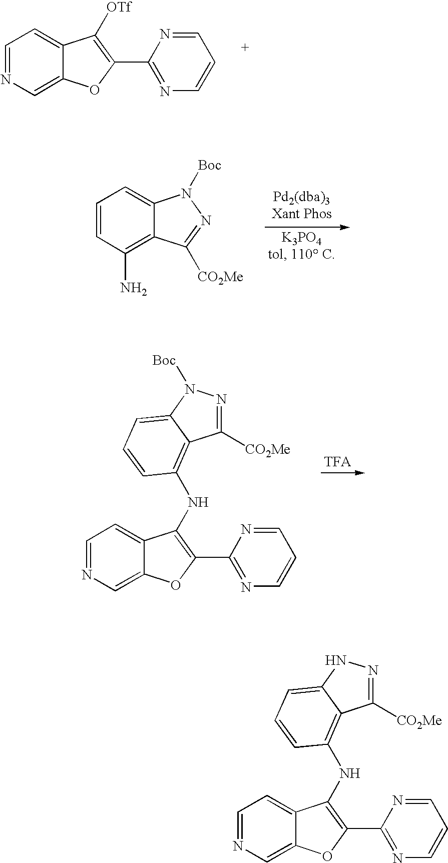

Step A: Preparation of 1-tert-butyl 3-methyl 4-(2-(pyrimidin-2-yl)furo[2,3-c]pyridin-3-ylamino)-1H-indazole-1,3-dicarboxylate: A mixture of 2-(pyrimidin-2-yl)furo[2,3-c]pyridin-3-yl trifluoromethanesulfonate (98 mg, 0.28 mmol) and 1-tert-butyl 3-methyl 4-amino-1H-indazole-1,3-dicarboxylate (77 mg, 0.26 mmol) were taken up in toluene (5 mL) and degassed with Ar for 15 minutes. XantPhos (32 mg, 0.06 mmol), Pd₂(dba)₃ (25 mg, 0.03 mmol) and K₃PO₄ (123 mg, 0.6 mmol) were added to the mixture. The mixture was degassed for an additional 15 minutes and heated to a temperature of 110° C. for 13 hours. The reaction was cooled, diluted with CH₂Cl₂, and filtered through GF/F paper. The filtrate was purified by silica gel chromatography (eluting with ethyl acetate) to afford 1-tert-butyl 3-methyl 4-(2-(pyrimidin-2-yl)furo[2,3-c]pyridin-3-ylamino)-1H-indazole-1,3-dicarboxylate (19 mg, 15%) as an oil. MS (APCI-neg) M-Boc−1=385.2.

Step B: Preparation of methyl 4-(2-(pyrimidin-2-yl)furo[2,3-c]pyridin-3-ylamino)-1H-indazole-3-carboxylate: A solution of 1-tert-butyl 3-methyl 4-(2-(pyrimidin-2-yl)furo[2,3-c]pyridin-3-ylamino)-1H-indazole-1,3-dicarboxylate (19 mg, 0.04 mmol) in CH₂Cl₂ (4 mL) was treated with TFA (2 mL) at ambient temperature. The reaction was stirred for 1 hour, and the volatiles were removed on a rotovap. The residue was partitioned between saturated aqueous NaHCO₃ and CH₂Cl₂, and the layers were separated. The organic layer was dried over Na₂SO₄, filtered and concentrated. The crude product was purified by silica gel chromatography to afford methyl 4-(2-(pyrimidin-2-yl)furo[2,3-c]pyridin-3-ylamino)-1H-indazole-3-carboxylate (7 mg, 46%) as a solid. MS (APCI-pos) M+1=387.3.

### Example 47

N4-(7-chloro-2-(pyrimidin-2-yl)furo[2,3-c]pyridin-3-yl)-1H-indazole-4,7-diamine

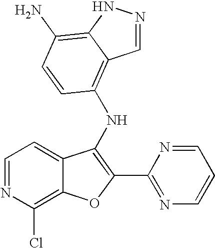

A suspension of 7-chloro-N-(7-chloro-2-(pyrimidin-2-yl)furo[2,3-c]pyridin-3-yl)-1H-indazol-4-amine (0.14 g, 0.35 mmol) in dioxane (4.0 mL) and 28% ammonium solution (6.0 mL) was heated in a steel bomb at 170° C. for 20 hours. The reaction mixture was cooled to room temperature. The volatiles were removed via rotovap, and the crude residue was purified by silica gel chromatography, eluting with hexanes/ethyl acetate (4:1), hexanes/ethyl acetate (2:1), hexanes/ethyl acetate (1:4), to afford the title compound (10 mg, 8%) as a solid. ¹H NMR (400 MHz, CDCl₃) δ 8.8 (m, 3H), 8.1 (s, 1H), 7.5 (d, J=5.6 Hz, 1H), 7.2 (t, J=4.6 Hz, 1H), 6.9 (d, J=5.6 Hz, 1H), 6.8 (d, J=7.8 Hz, 1H), 6.7 (d, J=7.8 Hz, 1H). MS (APCI-pos) M+1=378.3, 380.3.

### Example 48

### N-(7-chloro-6-methyl-1H-indazol-4-yl)-2-(pyrimidin-2-yl)furo[2,3-c]pyridin-3-amine

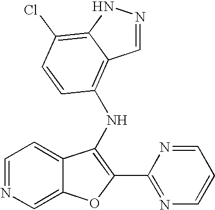

Step A: Preparation of 2,5-dimethyl-3-nitroaniline: The title compound was prepared as described in Example 32, Steps A and B, substituting 1,4-dimethyl-2-nitrobenzene for 4-chloro-1-methyl-2-nitrobenzene to give the title compound as a solid.

Step B: Preparation of 2-chloro-3,6-dimethyl-5-nitroaniline: 2,5-Dimethyl-3-nitrobenzenamine (0.415 g, 2.50 mmol) and n-chlorosuccinimide (0.367 g, 2.75 mmol) were dissolved in DMF (30 mL) and heated to 80° C. for 1 hour. The mixture was then cooled to room temperature. Water (100 mL) was added, and the resulting precipitated solid was collected by filtration and dried under high vacuum. Purification by silica gel chromatography provided the title compound (0.143 g, 29%) as a solid.

Step C: Preparation of 7-chloro-6-methyl-4-nitro-1H-indazole: The title compound was prepared as described in Example 7, Step C, substituting 2,6-dimethyl-3-nitroaniline for 2-chloro-3,6-dimethyl-5-nitroaniline to give the title compound as a solid. MS (APCI-neg) M−1=210.2.

Step D: Preparation of 7-chloro-6-methyl-4-nitro-1-((2-(trimethylsilyl)ethoxy)methyl)-1H-indazole: 7-Chloro-6-methyl-4-nitro-1H-indazole (0.102 g, 0.482 mmol) was dissolved in THF (4.0 mL) and cooled to 0° C. NaOtBu (0.0556 g, 0.578 mmol) was added and the reaction was stirred at 0° C. for 30 minutes. (2-(Chloromethoxy)ethyl)trimethylsilane (0.102 mL, 0.578 mmol) was added, and the reaction was warmed to room temperature for 1 hour. The reaction was partitioned between ethyl acetate and water. The organic layer was separated and dried over Na₂SO₄, concentrated, and filtered through a plug of SiO₂ with 8:1 Hex/EtOAc. The product was used directly in the next step.

Step E: Preparation of 7-chloro-6-methyl-1-((2-(trimethylsilyl)ethoxy)methyl)-1H-indazol-4-amine: The title compound was prepared as described in Example 32, Step D, substituting 7-chloro-6-methyl-4-nitro-1-((2-(trimethylsilyl)ethoxy)methyl)-1H-indazole for 1-(6-chloro-4-nitro-1H-indazol-1-yl)ethanone to give the title compound as a solid. ¹H NMR (400 MHz, CDCl₃) δ 7.96 (s, 1H), 6.36 (s, 1H), 6.08 (s, 2H), 4.10 (br s, 2H), 3.62-3.64 (m, 2H), 2.47 (s, 3H), 0.95-0.99 (m, 2H), 0.00 (s, 9H).

Step F: N-(7-chloro-6-methyl-1-((2-(trimethylsilyl)ethoxy)methyl)-1H-indazol-4-yl)-2-(pyrimidin-2-yl)furo[2,3-c]pyridin-3-amine: The title compound was prepared as described in Example 15, Step D, substituting 7-chloro-6-methyl-1-((2-(trimethylsilyl)ethoxy)methyl)-1H-indazol-4-amine for tert-butyl 4-amino-3-chloro-1H-indazole-1-carboxylate to give the title compound as a solid. MS (APCI-pos) M+1=507.2, 509.1.

Step G: Preparation of N-(7-chloro-6-methyl-1H-indazol-4-yl)-2-(pyrimidin-2-yl)furo[2,3-c]pyridin-3-amine: 7-Chloro-6-methyl-N-(2-(pyrimidin-2-yl)furo[2,3-c]pyridin-3-yl)-1-((2-(trimethylsilyl)ethoxy)methyl)-1H-indazol-4-amine (0.0066 g, 0.013 mmol) was dissolved in 10:1 MeOH/concentrated HCl (1 mL) and heated to 50° C. for 1 hour. The solution was concentrated and purified by silica gel chromatography to provide the title compound (0.0013 g, 24%) as a white solid. MS (APCI-pos) M+1=377.3.

### Example 49

### N-(7-fluoro-1H-indazol-4-yl)-2-(pyrimidin-2-yl)furo[2,3-c]pyridin-3-amine

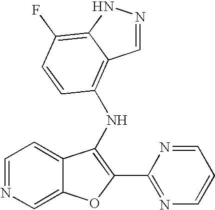

Step A: Preparation N-(6-fluoro-2-methyl-3-nitrophenyl)acetamide: The title compound was prepared as described in Example 32, Step A, substituting N-(2-fluoro-6-methylphenyl)acetamide for 4-chloro-1-methyl-2-nitrobenzene to give the title compound as a solid. MS (APCI-neg) M−1=211.2.

Step B: Preparation of 7-fluoro-4-nitro-1H-indazole: The title compound was prepared as described in Example 7, Step C, substituting N-(6-fluoro-2-methyl-3-nitrophenyl)acetamide for 2,6-dimethyl-3-nitroaniline to give the title compound as a solid. MS (APCI-neg) M−1=180.2.

Step C: Preparation of 7-fluoro-2-(methoxymethyl)-4-nitro-2H-indazole: 7-Fluoro-4-nitro-1H-indazole (0.020 g, 0.110 mmol) was dissolved in THF (3.0 mL) and cooled to 0° C. NaOtBu (0.0127 g, 0.133 mmol) was added, and the reaction was stirred at 0° C. for 30 minutes. Chloro(methoxy)methane (0.0101 mL, 0.133 mmol) was added, and the reaction was warmed to room temperature for 1 hour. The reaction was partitioned between ethyl acetate and water. The organic layer was separated, dried over Na₂SO₄ and concentrated. Purification by silica gel chromatography provided the title compound (0.011 g, 44%) as a solid. MS (APCI-neg) M−1=225.0.

Step D: Preparation of 7-fluoro-2-(methoxymethyl)-2H-indazol-4-amine: The title compound was prepared as described in Example 32, Step D, substituting 7-fluoro-2-(methoxymethyl)-4-nitro-2H-indazole for 1-(6-chloro-4-nitro-1H-indazol-1-yl)ethanone to give the title compound as a solid. ¹H NMR (400 MHz, CDCl₃) δ 8.07 (s, 1H), 6.51-6.56 (m, 1H), 6.28-6.30 (m, 1H), 5.64 (s, 2H), 4.17 (bs, 2H), 3.39 (s, 3H).

Step E: N-(7-fluoro-2H-indazol-4-yl)-2-(pyrimidin-2-yl)furo[2,3-c]pyridin-3-amine: The title compound was prepared as described in Example 15, Step D, substituting 7-fluoro-2-(methoxymethyl)-2H-indazol-4-amine for tert-butyl 4-amino-3-chloro-1H-indazole-1-carboxylate to give the title compound as a solid. MS (APCI-pos) M+1=507.2, 509.1.

Step F: Preparation of N-(7-fluoro-1H-indazol-4-yl)-2-(pyrimidin-2-yl)furo[2,3-c]pyridin-3-amine: N-(7-Fluoro-2-(methoxymethyl)-2H-indazol-4-yl)-2-(pyrimidin-2-yl)furo[2,3-c]pyridin-3-amine (0.0125 g, 0.03202 mmol) was dissolved in 6N HCl (2 mL) and heated to 60° C. for 30 minutes. The reaction was partitioned between ethyl acetate and saturated aqueous NaHCO₃. The organic layer was separated, washed with water, dried over Na₂SO₄ and concentrated. MS (APCI-pos) M+1=347.3.

While the invention has been described in conjunction with the enumerated embodiments, it will be understood that they are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. A compound selected from Formula I: 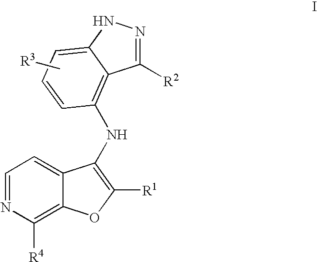
and stereoisomers and pharmaceutically acceptable salts thereof, wherein:
R¹ is selected from H, F, Cl, Br, I, —C(═O)R^{a}, —C(═O)OR^{b}, —C(═O)NR^{b}R^{c}, NR^{b}R^{c}, C₁-C₆ alkyl, C₅-C₈ aryl, C₃-C₈ carbocycle, 5 to 8 member heterocyclyl and 5 to 8 member heteroaryl, wherein said alkyl, aryl, carbocycle, heterocyclyl and heteroaryl are optionally substituted with one or more groups selected from F, Cl, Br, I, R^{d}, —OR^{d}, —COOR^{d}, —C(═O)NR^{d}R^{e}, —N(R^{d})C(═O)R^{e} and —NR^{d}R^{e};
R² is selected from H, F, Cl, Br, I, optionally substituted C₁-C₆ alkyl and —(X)R^{f}, wherein X is O, NH or C(═O), and wherein the alkyl is optionally substituted by one or more groups selected from —OR^{g}, —COOR^{g}, —C(═O)NR^{g}R^{h} and —NR^{g}R^{h};
R³ is one to three substituents independently selected from H, F, Cl, Br, I, CF₃, NH₂ and C₁-C₆ alkyl;
R⁴ is selected from H, F, Cl, Br, I, —NRⁱR^{j} and —ORⁱ;
R^{a} is selected from H, F, Cl, Br, I and C₁-C₆ alkyl, wherein the alkyl is optionally substituted with —NR^{m}Rⁿ or —OR^{m};
R^{b} and R^{c} are selected from H, C₁-C₆ alkyl and —(CR^{k}R^{l})ₜ-heteroaryl, wherein the heteroaryl has 5 to 8 members and the alkyl or heteroaryl are optionally substituted with (CR^{k}R^{l})ₜNR^{m}Rⁿ or —(CR^{k}R^{l})ₜOR^{m}, or
R^{b} and R^{c} together with the nitrogen to which they are attached form an optionally substituted 5 to 8 member heterocycle or 5 to 8 member heteroaryl, wherein the heterocycle or heteroaryl are optionally substituted with C₁-C₆ alkyl, —(CR^{k}R^{l})ₜNR^{m}Rⁿ or —(CR^{k}R^{l})ₜOR^{m};
R^{d} and R^{e} are independently selected from H or C₁-C₆ alkyl, wherein the alkyl is optionally substituted with —NR^{m}Rⁿ or —OR^{m}, or
R^{d} and R^{e} together with the nitrogen to which they are attached form an optionally substituted 5 to 8 member heterocycle or 5 to 8 member heteroaryl, wherein the heterocycle or heteroaryl are optionally substituted with C₁-C₆ alkyl, —(CR^{k}R^{l})ₜNR^{m}Rⁿ or —(CR^{k}R^{l})ₜOR^{m};
R^{f} is selected from H, C₁-C₄ alkyl, OR^{m} and NR^{m}Rⁿ, wherein the alkyl is optionally substituted with one or more groups selected from —OR^{m}, —COOR^{m}, —C(═O)NR^{m}Rⁿ and —NR^{m}Rⁿ;
R^{g} and R^{h} are independently selected from H, C₁-C₆ alkyl or a 5 to 8 member heterocyclyl, wherein the alkyl or heterocyclyl is optionally substituted with C₁-C₆ alkyl, —(CR^{k}R^{l})ₙNR^{m}Rⁿ or —(CR^{k}R^{l})ₜOR^{m};
Rⁱ and R^{j} are H, C₁-C₆ alkyl, —C(═O)R^{m}, —C(═O)OR^{m}, —S(O)₂NR^{m}Rⁿ, wherein the alkyl is optionally substituted with —NR^{m}Rⁿ or —OR^{m};
R^{k} and R^{l} are independently selected from H or C₁-C₆ alkyl;
R^{m} and Rⁿ are H or C₁-C₆ alkyl, or
R^{m} and Rⁿ together with the atom to which they are attached form an optionally substituted 5 to 8 member heterocycle or 5 to 8 member heteroaryl, wherein the heterocycle or heteroaryl are optionally substituted with F, Cl, Br, I or C₁-C₆ alkyl; and
t is 0, 1, 2, 3 or 4.

2. (canceled)

3. The compound of claim 1, wherein R⁴ is H or Cl.

4. (canceled)

5. The compound of claim 3, wherein R³ is H, Cl, F methyl or NH₂.

6. (canceled)

7. (canceled)

8. (canceled)

9. (canceled)

10. (canceled)

11. (canceled)

12. (canceled)

13. (canceled)

14. (canceled)

15. The compound of claim 5, wherein R¹ is selected from H, —C(═O)OEt,

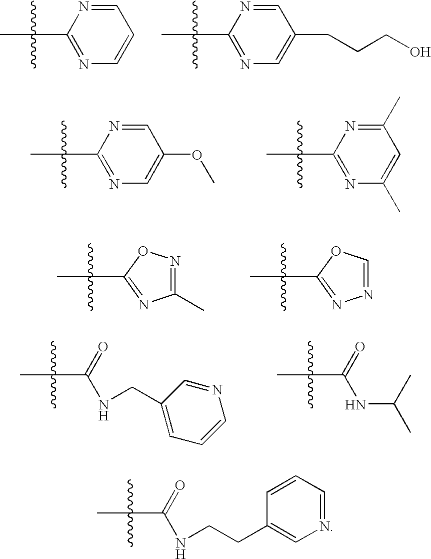

16. (canceled)

17. (canceled)

18. (canceled)

19. (canceled)

20. (canceled)

21. (canceled)

22. (canceled)

23. The compound of claim 15, wherein R² is H, Cl, ethyl, —CH₂CH₃C(═O)OH, —CH₂CH₂CH₂OH, —CH₂CH₂CH₂NH₂, —CH₂CH₂CH₂NHCH₃, —CH₂CH₂CH₂N(CH₃)₂ or —C(═O)OCH₃.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. A compound selected from Formula IIa: 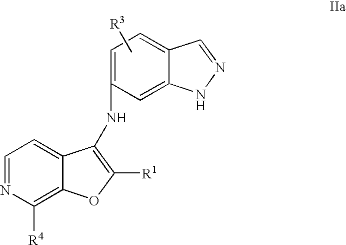
and stereoisomers and pharmaceutically acceptable salts thereof, wherein:
R¹ is selected from H, F, Cl, Br, I, —C(═O)R^{a}, —C(═O)OR^{b}, —C(═O)NR^{b}R^{c}, NR^{b}R^{c}, C₁-C₆ alkyl, C₅-C₈ aryl, C₃-C₈ carbocycle, 5 to 8 member heterocyclyl and 5 to 8 member heteroaryl, wherein said alkyl, aryl, carbocycle, heterocyclyl and heteroaryl are optionally substituted with one or more groups selected from F, Cl, Br, I, R^{d}, —OR^{d}, —COOR^{d}, —C(═O)NR^{d}R^{e}, —N(R^{d})C(═O)R^{e} and —NR^{d}R^{e};
R³ is one to three substituents independently selected from H, F, Cl, Br, I, CF₃, NH₂ and C₁-C₆ alkyl;
R⁴ is selected from H, F, Cl, Br, I, —NRⁱR^{j} and —ORⁱ;
R^{a} is selected from H, F, Cl, Br, I and C₁-C₆ alkyl, wherein the alkyl is optionally substituted with —NR^{m}Rⁿ or —OR^{m};
R^{b} and R^{c} are selected from H, C₁-C₆ alkyl and —(CR^{k}R^{l})ₜ-heteroaryl, wherein the heteroaryl has 5 to 8 members and the alkyl or heteroaryl are optionally substituted with —(CR^{k}R^{l})ₜNR^{m}Rⁿ or —(CR^{k}R^{l})ₜOR^{m}, or
R^{b} and R^{c} together with the nitrogen to which they are attached form an optionally substituted 5 to 8 member heterocycle or 5 to 8 member heteroaryl, wherein the heterocycle or heteroaryl are optionally substituted with C₁-C₆ alkyl, —(CR^{k}R^{l})ₜNR^{m}Rⁿ or —(CR^{k}R^{l})ₜOR^{m};
R^{d} and R^{e} are independently selected from H or C₁-C₆ alkyl, wherein the alkyl is optionally substituted with —NR^{m}Rⁿ or —OR^{m}, or
R^{d} and R^{e} together with the nitrogen to which they are attached form an optionally substituted 5 to 8 member heterocycle or 5 to 8 member heteroaryl, wherein the heterocycle or heteroaryl are optionally substituted with C₁-C₆ alkyl, —(CR^{k}R^{l})ₜNR^{m}Rⁿ or —(CR^{k}R^{l})ₜOR^{m};
Rⁱ and R^{j} are H, C₁-C₆ alkyl, —C(═O)R^{m}, —C(═O)OR^{m}, —S(O)₂NR^{m}Rⁿ, wherein the alkyl is optionally substituted with —NR^{m}Rⁿ or —OR^{m};
R^{k} and R^{l} are independently selected from H or C₁-C₆ alkyl;
R^{m} and Rⁿ are H, F, Cl, Br, I, OH, C(═O)OH or C₁-C₆ alkyl, or
R^{m} and Rⁿ together with the atom to which they are attached form an optionally substituted 5 to 8 member heterocycle or 5 to 8 member heteroaryl, wherein the heterocycle or heteroaryl are optionally substituted with F, Cl, Br, I or C₁-C₆ alkyl; and
t is 0, 1, 2, 3 or 4.

36. A compound of claim 35 having the Formula II: 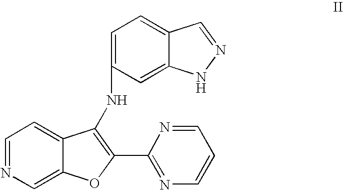
and stereoisomers and pharmaceutically acceptable salts thereof.

37. A compound selected from Formula IIIa: 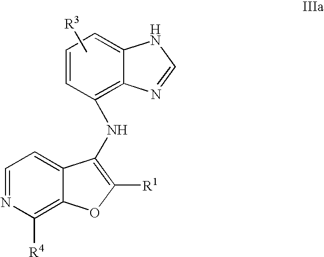
and stereoisomers and pharmaceutically acceptable salts thereof, wherein:
R¹ is selected from H, F, Cl, Br, I, —C(═O)R^{a}, —C(═O)OR^{b}, —C(═O)NR^{b}R^{c}, NR^{b}R^{c}, C₁-C₆ alkyl, C₅-C₈ aryl, C₃-C₈ carbocycle, 5 to 8 member heterocyclyl and 5 to 8 member heteroaryl, wherein said alkyl, aryl, carbocycle, heterocyclyl and heteroaryl are optionally substituted with one or more groups selected from F, Cl, Br, I, R^{d}, —OR^{d}, —COOR^{d}, —C(═O)NR^{d}R^{e}, —N(R^{d})C(═O)R^{e} and —NR^{d}R^{e};
R³ is one to three substituents independently selected from H, F, Cl, Br, I, CF₃, NH₂ and C₁-C₆ alkyl;
R⁴ is selected from H, F, Cl, Br, I, —NRⁱR^{j} and —ORⁱ;
R^{a} is selected from H, F, Cl, Br, I and C₁-C₆ alkyl, wherein the alkyl is optionally substituted with —NR^{m}Rⁿ or —OR^{m};
R^{b} and R^{c} are selected from H, C₁-C₆ alkyl and —(CR^{k}R^{l})ₜ-heteroaryl, wherein the heteroaryl has 5 to 8 members and the alkyl or heteroaryl are optionally substituted with —(CR^{k}R^{l})ₜNR^{m}Rⁿ or —(CR^{k}R^{l})ₜOR^{m}, or
R^{b} and R^{c} together with the nitrogen to which they are attached form an optionally substituted 5 to 8 member heterocycle or 5 to 8 member heteroaryl, wherein the heterocycle or heteroaryl are optionally substituted with C₁-C₆ alkyl, —(CR^{k}R^{l})ₜNR^{m}Rⁿ or —(CR^{k}R^{l})ₜOR^{m};
R^{d} and R^{e} are independently selected from H or C₁-C₆ alkyl, wherein the alkyl is optionally substituted with —NR^{m}Rⁿ or —OR^{m}, or
R^{d} and R^{e} together with the nitrogen to which they are attached form an optionally substituted 5 to 8 member heterocycle or 5 to 8 member heteroaryl, wherein the heterocycle or heteroaryl are optionally substituted with C₁-C₆ alkyl, —(CR^{k}R^{l})ₜNR^{m}Rⁿ or —(CR^{k}R^{l})ₜOR^{m};
Rⁱ and R^{j} are H, C₁-C₆ alkyl, —C(═O)R^{m}, —C(═O)OR^{m}, —S(O)₂NR^{m}Rⁿ, wherein the alkyl is optionally substituted with —NR^{m}Rⁿ or —OR^{m};
R^{k} and R^{l} are independently selected from H or C₁-C₆ alkyl;
R^{m} and Rⁿ are H, F, Cl, Br, I, OH, C(═O)OH or C₁-C₆ alkyl, or
R^{m} and Rⁿ together with the atom to which they are attached form an optionally substituted 5 to 8 member heterocycle or 5 to 8 member heteroaryl, wherein the heterocycle or heteroaryl are optionally substituted with F, Cl, Br, I or C₁-C₆ alkyl; and
t is 0, 1, 2, 3 or 4.

38. A compound of claim 37 having the Formula III: 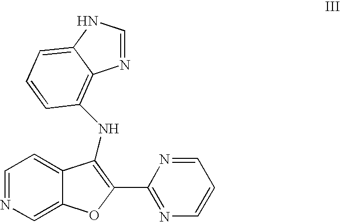
and stereoisomers and pharmaceutically acceptable salts thereof.

39. A method of preventing or treating a disease or disorder modulated by Raf kinases, comprising administering to a mammal in need of such treatment an effective amount of a compound of claim 1.

40. A method of preventing or treating cancer, comprising administering to a mammal in need of such treatment an effective amount of a compound of claim 1, alone or in combination with one or more additional compounds having anti-cancer properties.

41. A method of treating a hyperproliferative disease in a mammal comprising administering a therapeutically effective amount of a compound of claim 1 to the mammal.

42. (canceled)

43. A pharmaceutical composition comprising a compound of claim 1 or a pharmaceutically acceptable salt thereof, and a pharmaceutically acceptable carrier or excipient.

44. A process for preparing compounds of Formula 5: 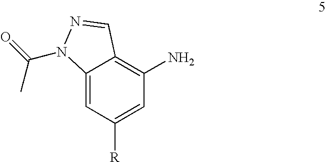
wherein R is H, F, Cl, Br, I, C₁-C₆ alkyl or C₁-C₆ alkoxy, comprising:
(a) reacting a substituted 2-nitrotoluene of Formula 1: 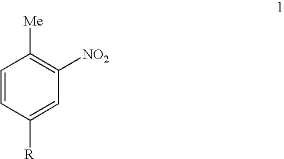
with a nitrating reagent to provide a substituted 2,6-dinitrotoluene of Formula 2: 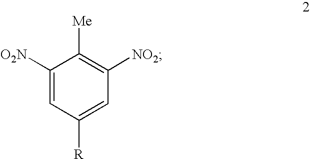
(b) selectively reducing the substituted 2,6-dinitrotoluene of Formula 2 to provide an aminotoluene of Formula 3: 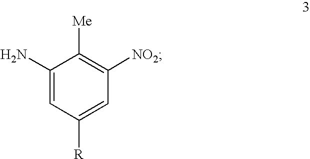
(c) converting the aminotoluene of Formula 3 to the corresponding nitroindazoles of Formula 4: 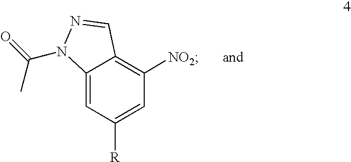
(d) reducing the nitroindazole of Formula 4 to give the 6-substituted indazole of Formula 5.
